(19) United States
(12) Patent Application Publication
(10) Pub. No.: US 2025/0283816 A1
(43) Pub. Date: Sep. 11, 2025

(54) COMPOSITIONS FOR DETECTION OF FLUOROCARBONS AND RELATED ARTICLES, SYSTEMS, AND METHODS

(71) Applicant: Massachusetts Institute of Technology, Cambridge, MA (US)

(72) Inventors: Timothy Manning Swager, Newton, MA (US); Alberto Concellon, Boston, MA (US)

(73) Assignee: Massachusetts Institute of Technology, Cambridge, MA (US)

(21) Appl. No.: 19/134,429

(22) PCT Filed: Dec. 1, 2023
(86) PCT No.: PCT/US2023/082090

§ 371 Date: May 30, 2025

Related U.S. Application Data
(60) Provisional application No. 63/385,728, filed on Dec. 1, 2022.
(60) Provisional application No. 63/503,979, filed on May 24, 2023.

(51) Int. Cl. G01N 21/64; G01N 21/78; G01N 33/18

(52) U.S. Cl. CPC G01N 21/6428; G01N 21/6447; G01N 21/78; G01N 33/1826; G01N 2021/6439

(57) ABSTRACT

Compositions, articles, systems, and methods for detection of fluorocarbons are generally described. In certain embodiments, for example, a sensing material that comprises a coupled-multichromophore is described. The coupled-multichromophore may be capable of energy transport and/or diffusion between individual sites of the coupled-multichromophore. In certain embodiments, for example, the coupled-multichromophore comprises individual sites that are linked through delocalized orbitals such that the coupled-multichromophore is capable of energy transport and/or diffusion through each individual site of the coupled-multichromophore. The sensing material comprising the coupled-multichromophore may be configured to detect the presence of an analyte, such as a fluoroalkyl species (e.g., a per- and/or polyfluoroalkyl substance). For example, in some embodiments, the coupled-multichromophore comprises at least one chromophore that displays a change in electromagnetic radiation (e.g., light) emission in response to a presence of the analyte. The change in electromagnetic radiation emission may be detected to determine the presence of the analyte.

### RELATED APPLICATIONS

This application claims priority under 35 U.S.C. § 119 (e) to U.S. Provisional Patent Application No. 63/385,728, filed Dec. 1, 2022, and entitled “Detection of Fluorocarbon Compounds,” and to U.S. Provisional Application No. 63/503,979, filed May 24, 2023, and entitled “Detection of PFAS,” both of which are incorporated herein by reference in their entirety for all purposes.

### TECHNICAL FIELD

Compositions, articles, systems, and methods for detection of fluorocarbons are generally described.

### BACKGROUND

Per- and polyfluoroalkyl substances (PFAS) are “forever chemicals” and their high chemical stability allows them to slowly accumulate over time in the environment as well as in living systems. Fluorinated materials have useful properties and are widely used in consumer products (e.g., food packaging, nonstick cookware, and lubricants) and as additives in fire-fighting foams, cleaning products, and personal care products. Studies conducted by the Center of Disease Control and Prevention (CDC) revealed that most people in the United States have been exposed to PFAS concentrations that may lead to detrimental health outcomes, such as thyroid disease, liver damage, reduced fertility, and even certain types of cancer. There are multiple types of PFAS, however “long-chain” perfluoroalkyl carboxylic acids (CₙF₂ₙ₊₁COOH, n≥7) and perfluoroalkyl sulfonic acids (CₙF₂ₙ₊₁SO₃H, n≥6) show particular resistance to degradation and are more bioaccumulative than their “short-chain” analogues. In response, the U.S. Environmental Protection Agency (EPA) released a health advisory level in 2016 of 70 ng·L⁻¹ (70 ppt) for a combined concentration of perfluorooctanoic acid (PFOA) and perfluorooctane sulfonate (PFOS) in drinking water. This health advisory limit was revised in 2022, and the current drinking water health advisories are 0.02 ppt for PFOA and 0.004 ppt for PFOS. Although these updated advisory levels are presently only interim limits, they are cautionary that some negative effects may occur with PFOA and PFOS concentrations in water that are at ultra-trace levels that are challenging to measure throughout distributed water infrastructures.

Current EPA methods for the detection of PFAS at the ng/L range rely on combinations of liquid chromatography and mass spectroscopy. Although these methods provide accuracy and sensibility, they are cost-prohibitive and require specialized laboratories with well-trained personnel. Recent research efforts have been focused on developing fast, portable, user-friendly, and low-cost detection methods that will allow for continuous environmental monitoring. However, few sensors are suitable for on-site detection, and they further lack sufficient sensitivity and/or selectivity. In addition to the ultra-low concentrations of PFAS in water, the complexity of real water samples, which usually contain various ions, biopolymers, humic acids, and/or organic oils or surfactants, make PFAS detection, monitoring, and mitigation challenging.

Accordingly, improved compositions, articles, systems, and methods for detection of fluorocarbons are needed.

### SUMMARY

Compositions for detection of fluorocarbons, and related articles, systems, and methods, are generally described. The subject matter of the present invention involves, in some cases, interrelated products, alternative solutions to a particular problem, and/or a plurality of different uses of one or more systems and/or articles.

This Summary introduces a selection of concepts in simplified form that are described further below in the Detailed Description. This Summary neither identifies key or essential features, nor limits the scope, of the claimed subject matter.

According to certain embodiments, a sensing material is described. In some embodiments, the sensing material comprises a coupled-multichromophore comprising at least one chromophore that displays a change in electromagnetic radiation emission in response to a presence of a fluoroalkyl substance, wherein the coupled-multichromophore is capable of energy transport between individual sites of the coupled-multichromophore.

In certain embodiments, a sensing material comprises a coupled-multichromophore comprising at least one chromophore that displays a change in electromagnetic radiation emission in response to protonation of the coupled-multichromophore by a fluoroalkyl substance, wherein the coupled-multichromophore is capable of energy transport between individual sites of the coupled-multichromophore.

According to some embodiments, a sensing material comprises a coupled-multichromophore comprising at least two chromophores that display a ratiometric change in electromagnetic radiation emission in response to a presence of a fluoroalkyl substance, wherein the coupled-multichromophore is capable of energy transport between individual sites of the coupled-multichromophore.

In certain embodiments, a method of detecting a fluoroalkyl substance is described. In some embodiments, the method comprises exposing a coupled-multichromophore to a solution comprising the fluoroalkyl substance, and detecting a change in electromagnetic radiation emission of at least one chromophore of the coupled-multichromophore, wherein the at least one chromophore displays the change in electromagnetic radiation emission in response to a presence of the fluoroalkyl substance.

According to some embodiments, a method of detecting a fluoroalkyl substance comprises exposing a coupled-multichromophore to a solution comprising the fluoroalkyl substance, and detecting a change in electromagnetic radiation emission of at least one chromophore of the coupled-multichromophore, wherein the at least one chromophore displays the change in electromagnetic radiation emission in response to protonation of the coupled-multichromophore by the fluoroalkyl substance.

In certain embodiments, a method of detecting a fluoroalkyl substance comprises exposing a coupled-multichromophore to a solution comprising the fluoroalkyl substance, and detecting a ratiometric change in electromagnetic radiation emission of at least two chromophores of the coupled-multichromophore, wherein the at least two chromophores display the ratiometric change in electromagnetic radiation emission in response to a presence of the fluoroalkyl substance.

According to certain embodiments, an article is described. In some embodiments, the article comprises a sensing material comprising a coupled-multichromophore comprising at least one chromophore that displays a change in electromagnetic radiation emission in response to a presence of a fluoroalkyl substance, wherein the coupled-multichromophore is capable of energy transport between individual sites of the coupled-multichromophore.

In certain embodiments, a system is described. In some embodiments, the system comprises a source of a solution and a sensing material comprising a coupled-multichromophore comprising at least one chromophore that displays a change in electromagnetic radiation emission in response to a presence of a fluoroalkyl substance, wherein the coupled-multichromophore is capable of energy transport between individual sites of the coupled-multichromophore.

One aspect of the disclosure herein is a method of detecting perfluoroalkyl and polyfluoroalkyl substances (PFAS) in an aqueous sample, the method comprising:
providing the aqueous sample containing the PFAS;
adding an amplifying fluorescent polymer (AFP) to the aqueous sample;
allowing the AFP and the PFAS to form an AFP-PFAS complex; and
detecting a presence of the PFAS in the aqueous sample by absorption and/or fluorescence spectra of the AFP-PFAS complex.

In one embodiment of the disclosed method, the AFP comprises a poly(p-phenylene ethynylene) backbone and/or a polyfluorene backbone.

In one embodiment of the disclosed method, the AFP comprises a backbone moiety comprising poly(p-phenylene ethynylene) (PPE), polyfluorene (PF), and/or fluorinated poly(p-phenylene ethynylene) (^{F}PPE).

In one embodiment of the disclosed method, the AFP comprises a PFAS selector, wherein the PFAS selector comprises pyridine (Py) having the structure:

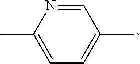

and/or
wherein the PFAS selector comprises a thiophene-functionalized pyridine (Py*) having the structure:

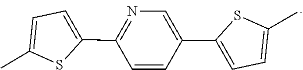

One aspect of the disclosure herein is a composition comprising a light-absorbing polymer and a dye, wherein the light-absorbing polymer and/or the dye comprise perfluoroalkane groups, wherein the perfluoroalkane groups comprise at least 25% w:w fluorine, and wherein the composition produces a sensing emission characteristic in response to a PFAS analyte.

In one embodiment of the disclosed composition, the dye is a small molecule or a polymer.

In one embodiment of the disclosed composition, the dye is selected from fluorous squaraine (F-Sq), fluorous oxazine (F-Ox), fluorous perylene bisimide (F-FBI), and mixtures and/or conjugates thereof.

In one embodiment of the disclosed composition, the light-absorbing polymer comprises a conjugated polymer comprising PPE (43% w:w fluorine) and/or ^{F}PPE (61% w:w fluorine).

In one embodiment of the disclosed composition, either or both of the light-absorbing polymer and the dye comprises a Brønsted acid.

In one embodiment of the disclosed composition, either or both of the light-absorbing polymer and the dye comprises a Brønsted base.

In one embodiment of the disclosed composition, the PFAS analyte is a Brønsted acid.

In one embodiment of the disclosed composition, the perfluoroalkane groups comprise more than 30%, 35%, 40%, or 50% w:w fluorine.

In one embodiment of the disclosed composition, the light-absorbing polymer is a conjugated polymer.

In one embodiment of the disclosed composition, the sensing emission characteristic in response to the PFAS analyte is a change in a ratio of two emissions.

In one embodiment of the disclosed composition, the sensing emission characteristic in response to the PFAS analyte is a change in an emission intensity at a particular wavelength.

In one embodiment of the disclosed composition, the sensing emission characteristic in response to the PFAS analyte is a change in intensity of an emission with a lifetime greater than 10 nanoseconds.

In one embodiment of the disclosed composition, the sensing emission characteristic in response to the PFAS analyte is an increase in emission intensity.

In one embodiment of the disclosed composition, the sensing emission characteristic in response to the PFAS analyte is a decrease in emission intensity.

In one embodiment of the disclosed composition, the sensing emission characteristic in response to the PFAS analyte is generated by an acidity of the perfluoroalkane groups.

In one embodiment of the disclosed composition, the sensing emission characteristic in response to the PFAS analyte originates with an exciplex.

In one embodiment of the disclosed composition, the sensing emission characteristic in response to the PFAS analyte is detected in an aqueous solution.

In one embodiment of the disclosed composition, the PFAS analyte is detected at less than 200 parts per billion in the aqueous solution.

One aspect of the disclosure herein is a system comprising the disclosed composition, wherein the system continuously monitors water for a presence of PFAS.

In one embodiment of the disclosed system, the system comprises a surface comprising the light absorbing polymer and the dye.

In one embodiment of the disclosed system, the system comprises a suspension of particles in water, wherein the particles comprise the light absorbing polymer and the dye.

In one embodiment of the disclosed system, the system further comprises a means for concentrating the PFAS analyte.

In one embodiment of the disclosed system, the system is capable of detecting the PFAS analyte in water at a concentration less than 1 part per billion.

In one embodiment of the disclosed system, the system is capable of detecting the PFAS analyte in water at a concentration less than 10 parts per trillion.

The following Detailed Description references the accompanying drawings which form a part of this application, and which show, by way of illustration, specific example implementations. Other implementations may be made without departing from the scope of the disclosure. Other advantages and novel features of the present invention will become apparent from the following Detailed Description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A shows, according to certain embodiments, a cross-sectional schematic diagram of a sensing material comprising a film comprising a coupled-multichromophore.

FIG. 1B shows, according to certain embodiments, a cross-sectional schematic diagram of a sensing material comprising a plurality of particles comprising a coupled-multichromophore.

FIG. 2 shows, according to certain embodiments, a cross-sectional schematic diagram of an article comprising a substrate and a sensing material disposed on at least a portion of the substrate.

FIG. 3A shows, according to certain embodiments, a cross-sectional schematic diagram of an article comprising a substrate and a sensing material disposed on at least a portion of the substrate, wherein the substrate and the sensing material comprise a plurality of pores.

FIG. 3B shows, according to certain embodiments, a top-view schematic diagram of the article shown in FIG. 3A.

FIG. 4 shows, according to certain embodiments, a cross-sectional schematic diagram of an article comprising a substrate and a sensing material disposed on at least a portion of the substrate, wherein the substrate comprises a particle.

FIGS. 5A-5B show, according to certain embodiments, a schematic diagram representing a method comprising exposing a film of a sensing material comprising a coupled-multichromophore to a solution comprising a fluoroalkyl substance.

FIGS. 6A-6B show, according to certain embodiments, a schematic diagram representing another method comprising exposing a film of a sensing material comprising a coupled-multichromophore to a solution comprising a fluoroalkyl substance.

FIGS. 7A-7B show, according to certain embodiments, a schematic diagram representing a method comprising exposing a plurality of particles of a sensing material comprising a coupled-multichromophore to a solution comprising a fluoroalkyl substance.

FIGS. 8A-8B show, according to certain embodiments, a schematic diagram representing a method comprising exposing a substrate and a sensing material comprising a coupled-multichromophore disposed on the substrate to a solution comprising a fluoroalkyl substance.

FIGS. 9A-9B show, according to certain embodiments, a schematic diagram representing another method comprising exposing a substrate and a sensing material comprising a coupled-multichromophore disposed on the substrate to a solution comprising a fluoroalkyl substance.

FIGS. 10A-10C show, according to certain embodiments, a schematic diagram representing a method comprising exposing a substrate and a sensing material comprising a coupled-multichromophore disposed on the substrate to a solution comprising a fluoroalkyl substance, wherein the substrate and the sensing material comprise a plurality of pores.

FIGS. 11A-11B show, according to certain embodiments, a schematic diagram representing a method comprising exposing a substrate and a sensing material comprising a coupled-multichromophore disposed on the substrate to a solution comprising a fluoroalkyl substance, wherein the substrate comprises a particle.

FIG. 12 shows, according to certain embodiments, a schematic diagram of a system comprising a source of a solution and a sensing material comprising a coupled-multichromophore.

FIG. 13 shows, according to certain embodiments, chemical structures of fluorous conjugated polymers (x=0.80, y=0.20) and a conceptual scheme of a mechanism for the detection of PFAS in water, wherein perfluorooctanoic acid (PFOA) diffusion from water to the polymer induces protonation of the pyridine moieties and triggers changes in the emission of the polymers.

FIG. 14A shows, according to certain embodiments, a synthetic route to highly fluorous polyfluorenes (x=0.80, y=0.20).

FIG. 14B shows, according to certain embodiments, a synthetic route to fluorous poly(p-phenylene ethynylenes) (x=0.80, y=0.20).

FIG. 15A shows, according to certain embodiments, absorption (dotted lines) and fluorescence (solid lines) spectra of PPE-Py in a benzotrifluoride solution, a spin-cast film of PPE-Py, and PPE-Py conjugated polymer nanoparticles (CPdots).

FIG. 15B shows, according to certain embodiments, absorption (dotted lines) and fluorescence (solid lines) spectra of ^{F}PPE-Py in a benzotrifluoride solution and a spin-cast film of ^{F}PPE-Py.

FIG. 15C shows, according to certain embodiments, absorption (dotted lines) and fluorescence (solid lines) spectra of PF-Py in a benzotrifluoride solution, a spin-cast film of PF-Py, and PF-Py CPdots.

FIG. 15D shows, according to certain embodiments, absorption (dotted lines) and fluorescence (solid lines) spectra of PPE-Py* in a benzotrifluoride solution, a spin-cast film of PPE-Py*, and PPE-Py*CPdots.

FIG. 15E shows, according to certain embodiments, absorption (dotted lines) and fluorescence (solid lines) spectra of ^{F}PPE-Py* in a benzotrifluoride solution and a spin-cast film of ^{F}PPE-Py*.

FIG. 15F shows, according to certain embodiments, absorption (dotted lines) and fluorescence (solid lines) spectra of PF-Py* in a benzotrifluoride solution, a spin-cast film of PF-Py*, and PF-Py* CPdots.

FIG. 16A shows, according to certain embodiments, fluorescence spectra of thin films of PPE-Py upon exposure to aqueous solutions of PFOA, and fluorescence photographs of the corresponding thin film.

FIG. 16B shows, according to certain embodiments, fluorescence spectra of thin films of PPE-Py* upon exposure to aqueous solutions of PFOA, and fluorescence photographs of the corresponding thin film.

FIG. 16C shows, according to certain embodiments, fluorescence spectra of thin films of PF-Py upon exposure to aqueous solutions of PFOA, and fluorescence photographs of the corresponding thin film.

FIG. 16D shows, according to certain embodiments, fluorescence spectra of thin films of PF-Py* upon exposure to aqueous solutions of PFOA, and fluorescence photographs of the corresponding thin film.

FIG. 17A shows, according to certain embodiments, changes in fluorescence intensity of PPE-Py thin films after exposure to PFOA in milliQ water, deionized (DI) water, and well water (average values of three different films, error bars represent standard deviations).

FIG. 17B shows, according to certain embodiments, changes in fluorescence intensity of PPE-Py* thin films after exposure to PFOA in milliQ water, DI water, and well water (average values of three different films, error bars represent standard deviations).

FIG. 17C shows, according to certain embodiments, changes in fluorescence intensity of PF-Py thin films after exposure to PFOA in milliQ water, DI water, and well water (average values of three different films, error bars represent standard deviations).

FIG. 17D shows, according to certain embodiments, changes in fluorescence intensity of PF-Py* thin films after exposure to PFOA in milliQ water, DI water, and well water (average values of three different films, error bars represent standard deviations).

FIG. 18A shows, according to certain embodiments, changes in fluorescence intensity of PPE-Py thin films after exposure to perfluorooctyl sulfonate (PFOS) in milliQ water and well water (average values of three different measurements, error bars represent standard deviations).

FIG. 18B shows, according to certain embodiments, changes in fluorescence intensity of PPE-Py* CPdots after exposure to PFOS in milliQ water and well water (average values of three different measurements, error bars represent standard deviations).

FIG. 19A shows, according to certain embodiments, dynamic light scattering (DLS) measurements of water dispersions of CPdots.

FIG. 19B shows, according to certain embodiments, a transmission electron microscopy (TEM) image of PPE-Py* CPdots.

FIG. 20A shows, according to certain embodiments, fluorescence spectra of PPE-Py CPdots upon exposure to aqueous solutions of PFOA, and the fluorescence photographs of the corresponding CPdots dispersion.

FIG. 20B shows, according to certain embodiments, fluorescence spectra of PPE-Py* CPdots upon exposure to aqueous solutions of PFOA, and the fluorescence photographs of the corresponding CPdots dispersion.

FIG. 20C shows, according to certain embodiments, fluorescence spectra of PF-Py CPdots upon exposure to aqueous solutions of PFOA, and the fluorescence photographs of the corresponding CPdots dispersion.

FIG. 20D shows, according to certain embodiments, fluorescence spectra of PF-Py* CPdots upon exposure to aqueous solutions of PFOA, and the fluorescence photographs of the corresponding CPdots dispersion.

FIG. 21A shows, according to certain embodiments, changes in fluorescence intensity of PPE-Py CPdots after exposure to PFOA in milliQ water and well water (average values of three different CPdots dispersions, error bars represent standard deviations).

FIG. 21B shows, according to certain embodiments, changes in fluorescence intensity of PPE-Py* CPdots after exposure to PFOA in milliQ water and well water (average values of three different CPdots dispersions, error bars represent standard deviations).

FIG. 21C shows, according to certain embodiments, changes in fluorescence intensity of PF-Py CPdots after exposure to PFOA in milliQ water and well water (average values of three different CPdots dispersions, error bars represent standard deviations).

FIG. 21D shows, according to certain embodiments, changes in fluorescence intensity of PF-Py* CPdots after exposure to PFOA in milliQ water and well water (average values of three different CPdots dispersions, error bars represent standard deviations).

FIG. 22 shows, according to certain embodiments, a table of molecular weight and polydispersity indices of synthesized polymers.

FIG. 23 shows, according to certain embodiments, a table of photophysical data.

FIG. 24 shows, according to certain embodiments, chemical structures of fluorous poly(p-phenylene ethynylenes) and fluorous dyes and a conceptual scheme of the mechanism for the detection of PFAS in water, wherein PFOA diffusion from water into the polymer disrupts π-π interactions between the dye and the conjugated polymer, interrupting the electron exchange-based electron transfer (ET).

FIG. 25A shows, according to certain embodiments, fluorescence spectra of PPE/F-Sq in thin film as a function of dye loading.

FIG. 25B shows, according to certain embodiments, fluorescence spectra of PPE/F-Sq in thin film upon exposure to aqueous solutions of PFOA.

FIG. 25C shows, according to certain embodiments, changes in thin film fluorescence intensity of PPE/F-Sq and ^{F}PPE/F-Sq after 1 hour exposure to PFOA in milliQ water (average values of three different films, errors bars represent standard deviations).

FIG. 26A shows, according to certain embodiments, changes in thin film fluorescence intensity of PPE and F-Sq after 1 hour exposure to aqueous solutions of PFOA in milliQ water (solid) and well water (patterned) (average values of three different films, errors bars represent standard deviations).

FIG. 26B shows, according to certain embodiments, changes in thin film fluorescence intensity of PPE and F-Sq after 1 hour exposure to aqueous solutions of PFOS in milliQ water (solid) and well water (patterned) (average values of three different films, errors bars represent standard deviations).

FIG. 27A shows, according to certain embodiments, DLS measurements of water dispersions of CPdots.

FIG. 27B shows, according to certain embodiments, a TEM image of PPE/F-Sq CPdots.

FIG. 27C shows, according to certain embodiments, fluorescence spectra of PPE/F-Sq CPdots as a function of dye loading.

FIG. 28A shows, according to certain embodiments, changes in CPdots fluorescence intensity of PPE and F-Sq after 1 hour exposure to aqueous solutions of PFOA in milliQ water (solid) and well water (patterned) (average values of three different CPdots dispersions, errors bars represent standard deviations).

FIG. 28B shows, according to certain embodiments, changes in CPdots fluorescence intensity of PPE and F-sq after 1 hour exposure to aqueous solutions of PFOS in milliQ water (solid) and well water (patterned) (average values of three different CPdots dispersions, errors bars represent standard deviations).

FIG. 29A shows, according to certain embodiments, absorbance (solid line) and fluorescence (dotted line) spectra of PPE and fluorous dye F-Sq.

FIG. 29B shows, according to certain embodiments, thin film fluorescence spectra of PPE/F-Sq dye formulations as a function of dye loading.

FIG. 29C shows, according to certain embodiments, absorbance (solid line) and fluorescence (dotted line) spectra of PPE and fluorous dye F-Ox.

FIG. 29D shows, according to certain embodiments, thin film fluorescence spectra of PPE/F-Ox dye formulations as a function of dye loading.

FIG. 29E shows, according to certain embodiments, absorbance (solid line) and fluorescence (dotted line) spectra of PPE and fluorous dye F-PBI.

FIG. 29F shows, according to certain embodiments, thin film fluorescence spectra of PPE/F-PBI dye formulations as a function of dye loading.

FIG. 30 shows, according to certain embodiments, thin film fluorescence spectra of PPE/Sq upon 1 hour exposure to aqueous solutions of octanoic acid.

FIG. 31 shows, according to certain embodiments, a proton nuclear magnetic resonance (¹H NMR) spectrum (400 MHz, 298K, CDCl₃) of PPE.

FIG. 32 shows, according to certain embodiments, a fluorine nuclear magnetic resonance (¹⁹F NMR) spectrum (376 MHz, 298K, CDCl₃) of PPE.

FIG. 33 shows, according to certain embodiments, a schematic of a synthetic route to fluorous poly(p-phenylene ethynylenes).

### DETAILED DESCRIPTION

Compositions, articles, systems, and methods for detection of fluorocarbons are generally described. In certain embodiments, for example, a sensing material that comprises a coupled-multichromophore is described. The coupled-multichromophore may be capable of energy transport and/or diffusion between individual sites of the coupled-multichromophore. In certain embodiments, for example, the coupled-multichromophore comprises individual sites that are linked through delocalized orbitals such that the coupled-multichromophore is capable of energy transport and/or diffusion through each individual site of the coupled-multichromophore. The sensing material comprising the coupled-multichromophore may be configured to detect the presence of an analyte, such as a fluoroalkyl species (e.g., a per- and/or polyfluoroalkyl substance). For example, in some embodiments, the coupled-multichromophore comprises at least one chromophore that displays a change in electromagnetic radiation (e.g., light) emission in response to a presence of the analyte. The change in electromagnetic radiation emission may, in some embodiments, be detected to determine the presence of the analyte. In certain embodiments, the presence of the analyte may be determined at ultra-trace (e.g., ppb or ppt) levels.

In certain embodiments, the coupled-multichromophore comprises a moiety that is capable of being protonated by the analyte. In response to protonation of the coupled-multichromophore by the analyte, at least one chromophore of the coupled-multichromophore may display the change in electromagnetic radiation (e.g., light) emission. In some embodiments, the change in electromagnetic radiation emission of the at least one chromophore is a wavelength shift of an emission peak to a lower energy emission. In accordance with certain embodiments, upon detecting the change in electromagnetic radiation emission of the at least one chromophore, the presence of the analyte is detected.

According to some embodiments, the coupled-multichromophore comprises a dye. In response to the analyte changing an organization of at least a portion of the coupled-multichromophore, at least two chromophores of the coupled-multichromophore may display a ratiometric change in electromagnetic radiation (e.g., light) emission. In certain embodiments, for example, the analyte displaces at least a portion of the dye from the coupled-multichromophore. In some embodiments, the ratiometric change in electromagnetic radiation emission comprises an increase in an emission peak of a first chromophore and a decrease of an emission peak of a second chromophore. In accordance with some embodiments, upon detecting the ratiometric change in electromagnetic radiation emission of the at least two chromophores, the presence of the analyte is detected.

The sensing material comprising the coupled-multichromophore may be in any of a variety of suitable forms. In some embodiments, for example, the sensing material comprising the coupled-multichromophore is a film (e.g., a thin film). In other embodiments, the sensing material comprising the coupled-multichromophore is a plurality of particles. In yet other embodiments, the sensing material comprising the coupled-multichromophore is disposed on (e.g., coated on) a substrate. The sensing material comprising the coupled-multichromophore disposed on the substrate may be in the form of a film, a filter, and/or a membrane. In some embodiments, the substrate is a particle and the sensing material comprising the coupled-multichromophore is disposed on (e.g., coated on) the particle. In certain embodiments, the sensing material is incorporated into a system comprising a source of a solution such that the solution flows from the source and is exposed to the sensing material.

Methods of detecting an analyte are also described herein. In some embodiments, for example, the sensing material comprising the coupled-multichromophore is exposed to a solution comprising an analyte. In certain embodiments, the analyte is detected by detecting a change in electromagnetic radiation (e.g., light) emission of at least one chromophore of the coupled-multichromophore, wherein the at least one chromophore of the coupled-multichromophore displays the change in electromagnetic radiation emission in response to a presence of the analyte.

According to certain embodiments, the sensing material comprises a coupled-multichromophore. As used herein, the term “coupled-multichromophore” refers to a material that includes at least a first individual site and a second individual site that are coupled together, wherein the material is capable of including or hosting multiple chromophores. The term “coupled” as used herein indicates some level of interaction between the individual sites of the coupled-multichromophore that allows excited states generated in the coupled-multichromophore to move between the individual sites. In some embodiments, each individual site of the coupled-multichromophore may be coupled such that each individual site is bound (e.g., covalently or otherwise chemically bound) together. In certain embodiments, for example, the coupled-multichromophore is or comprises a conjugated polymer.

As used herein, the term “individual site” refers to a portion of a material (e.g., the coupled-multichromophore) that is larger than a single atom, which is separated by at least one other atom from a different individual site, and which defines a chromophore. In some embodiments, for example, an individual site of the coupled-multichromophore comprises one or more repeating units of a polymer, a portion of a polymer, a portion of a repeating unit of a polymer, a moiety, a portion of a moiety, a small molecule (e.g., a dye), a portion of a small molecule, and the like. In some cases, these individual sites each include sufficient atomic makeup and combination of atoms so as to perform a particular function associated with the disclosure herein (such as, e.g., absorption and/or emission of electromagnetic radiation), and each individual site is separated by sufficient atomic makeup from another individual site such that each individual site can perform its own function largely independently of the other, even if those functions are interrelated. For example, in some embodiments, one individual site may be involved in absorption of electromagnetic radiation, and another individual site is emissive in responsive to that absorption, with energy (e.g., exciton and/or electron) transfer occurring between the individual sites. Of course, the atomic makeup, connectivity, and proximity of one individual site relative to another individual site (e.g., a nearest neighbor individual site) can affect the molecular and electronic structure of either or both individual sites, but not so much as to negate the individual site's function. This concept-different individual sites of a material that interact with electromagnetic radiation, and with each other, in this way—is well known and understood by those of ordinary skill in the art, although those of ordinary skill would not arrive at the arrangements of this disclosure and the arrangements as claimed, without the teachings and guidance of this disclosure. Examples of individual sites, and energy transfer between individual sites, are provided more fully below.

As used herein, the term “chromophore” refers to a chemical group of a molecule that absorbs electromagnetic radiation (e.g., light, or other electromagnetic radiation including ultraviolet, infrared, and/or radiation extending beyond or completely outside the realm of visible, ultraviolet, or infrared) at a specific frequency or frequency profile, range, or pattern, and emits electromagnetic radiation at a specific frequency or frequency profile, range, or pattern. In some cases, the absorption and emission of electromagnetic radiation by a chemical group of a molecule imparts subtractive and/or additive color to the molecule. In some embodiments, the frequency at which the chromophore absorbs electromagnetic radiation and the frequency at which the chromophore emits electromagnetic radiation may be similar, but the frequency at which the chromophore emits electromagnetic radiation may be lower than the frequency at which the chromophore absorbs electromagnetic radiation. For example, in some embodiments, a photon absorbed by a chromophore creates an excited state that releases some energy through vibrations, solvent reorganization, and/or other processes, and the emitted photon is lower in energy. In other embodiments, the frequency at which the chromophore absorbs electromagnetic radiation and the frequency at which the chromophore emits electromagnetic radiation have larger changes in frequencies, in which case the radiation pattern or range of the absorption frequency and the emission frequency may not overlap, but are determinably different. For example, in certain embodiments wherein energy is transferred from one chromophore that normally emits at high frequency to a chromophore that emits at lower frequency, a large change in the frequency can be observed. As used herein, the term “electromagnetic radiation” is given its ordinary meaning in the art and refers to waves of an electromagnetic field that propagate through space and carry momentum and electromagnetic radiant energy, including radio waves, microwaves, infrared, (visible) light, ultraviolet, X-rays, and gamma rays. In some embodiments, the coupled-multichromophore is preferably excited by ultraviolet and/or visible light.

According to some embodiments, the coupled-multichromophore is capable of energy transport and/or diffusion between individual sites of the coupled-multichromophore. In certain embodiments, for example, the coupled-multichromophore comprises at least two individual sites (e.g., at least two chromophores) that are that are linked through an atomic structural link providing delocalized orbitals. In some embodiments, the at least two individual sites are capable of energy (e.g., exciton and/or electron) transport and/or diffusion from a first individual site (e.g., a first chromophore) of the coupled-multichromophore to a second individual site (e.g., a second chromophore) of the coupled-multichromophore coupled to the first individual site. In certain embodiments, the first individual site directly neighbors the second individual site. In some embodiments, a coupled-multichromophore that is capable of energy transport between individual sites of the coupled-multichromophore may advantageously be used to detect an analyte of interest due to the rapidly diffusing excitons and/or electrons sampling each individual site of the coupled-multichromophore. In certain embodiments, for example, the analyte of interest may interact with the coupled-multichromophore (e.g., with an individual site of the coupled-multichromophore), thereby generating a lower energy trapping state that captures the rapidly diffusing exciton and/or electron.

In some embodiments, the coupled-multichromophore comprises at least one chromophore that displays a change in electromagnetic radiation (e.g., light) emission in response to a presence of the analyte. In certain embodiments, as described in greater detail herein, the analyte is a fluoroalkyl substance. In some embodiments, the analyte (e.g., fluoroalkyl substance) interacts with the coupled-multichromophore (e.g., with an individual site of the coupled-multichromophore), thereby generating the lower energy trapping state that captures the rapidly diffusing exciton and/or electron, which results in the coupled-multichromophore (e.g., an individual site of the coupled-multichromophore that has not interacted with the analyte and/or the individual site of the coupled-multichromophore that has interacted with the analyte) displaying the change in electromagnetic radiation emission.

According to some embodiments, the coupled-multichromophore comprises a polymer. For example, in certain embodiments, the coupled-multichromophore comprises a conjugated polymer. As used herein, the term “conjugated polymer” is given its ordinary meaning in the art and refers to a macromolecule characterized by a backbone chain of alternating double- and single-bonds such that overlapping p-orbitals create of a system of delocalized π-electrons. In certain embodiments, a coupled-multichromophore comprising a conjugated polymer may advantageously facilitate energy transport and/or diffusion between individual sites of the coupled-multichromophore. In some embodiments, as described herein in greater detail, a coupled-multichromophore comprising a conjugated polymer is an amplifying fluorescence polymer that is used in a sensor (e.g., a fluorescence sensor) to enhance the sensitivity of the sensor to an analyte (e.g., a fluoroalkyl substance).

In certain embodiments wherein the coupled-multichromophore comprises a polymer, one or more individual sites of the coupled-multichromophore comprise one or more repeating units of the polymer and/or a portion of one or more repeating units of the polymer. In some embodiments, for example, the coupled-multichromophore is capable of energy transport and/or diffusion between the one or more repeating units of the polymer and one or more other individual sites of the coupled-multichromophore (e.g., one or more other repeating units of the polymer).

According to certain embodiments, the polymer is porous. In some embodiments, the use of a coupled-multichromophore comprising a porous polymer advantageously facilitates absorption of an analyte (e.g., a fluoroalkyl substance) into the coupled-multichromophore. In certain embodiments, for example, a fluoroalkyl substance absorbs into the coupled-multichromophore comprising a porous polymer such that the fluoroalkyl substance diffuses into one or more pores of the porous polymer upon exposing the coupled-multichromophore to the fluoroalkyl substance.

According to certain embodiments, the porosity of the porous polymer can be characterized by the Brunauer-Emmett-Teller (BET) surface area of the porous polymer. The porous polymer may have any of a variety of suitable BET surface areas. In some embodiments, for example, the porous polymer has a BET surface area greater than or equal to 100 m²/g, greater than or equal to 200 m²/g, greater than or equal to 300 m²/g, greater than or equal to 400 m²/g, greater than or equal to 500 m²/g, greater than or equal to 600 m²/g, greater than or equal to 700 m²/g, greater than or equal to 800 m²/g, or greater than or equal to 900 m²/g. In certain embodiments, the porous polymer has a BET surface area less than or equal to 1,000 m²/g, less than or equal to 900 m²/g, less than or equal to 800 m²/g, less than or equal to 700 m²/g, less than or equal to 600 m²/g, less than or equal to 500 m²/g, less than or equal to 400 m²/g, less than or equal to 300 m²/g, or less than or equal to 200 m²/g. Combinations of the above recited ranges are possible (e.g., the porous polymer has a BET surface area greater than or equal to 100 m²/g and less than or equal to 1,000 m²/g, the porous polymer has a BET surface area greater than or equal to 400 m²/g and less than or equal to 600 m²/g). Other ranges are also possible. It is also possible that a polymer can have limited porosity (<50 m²/g) and still absorb an analyte, as described herein in greater detail.

The coupled-multichromophore may comprise any of a variety of suitable polymers. In some embodiments, for example, the polymer comprises a statistical polymer, a block polymer, copolymers thereof, and/or combinations thereof. In certain embodiments, the polymer comprises a backbone comprising an acrylate, a styrene, a vinyl-ether, a norborene, an arylene, cellulose, an arylene ether, an arylene amine, a diene, a siloxane, an alkene, conjugates thereof, and/or combinations thereof. In certain embodiments, the chromophores of the coupled-multichromophore are pendant to the backbone of the polymer.

According to certain embodiments, the polymer is a fluorescent polymer. In some embodiments, for example, the polymer is an amplifying fluorescent polymer.

According to some embodiments, the coupled-multichromophore comprises a polyarylene, a poly(arylene vinylene), a poly(thiophene), a poly(phenylene), a poly(fluorene), a poly(phenylene), a poly(arylene ethynylene), a poly(phenylene ethynylene), copolymers thereof, and/or combinations thereof. Other polymers are also possible. In some embodiments, for example, the coupled-multichromophore comprises a poly(arylene ether), which is not conjugated, but energy can migrate within the polymer.

In certain embodiments, the coupled-multichromophore comprises an assembly of small molecules. In certain embodiments, for example, the coupled-multichromophore comprises an oil (e.g., a hydrocarbon, a siloxane, a halocarbon, a fluorocarbon), an alkane (e.g., a fluoroalkane), a cyclodextrin, a calixarene, a cavitand, a triptycene, an iptycene, a Lewis acid, a Lewis base, a Brønsted base, a metal ion, a macrocycle, conjugates thereof, and/or combinations thereof. Other small molecules are also possible.

The coupled-multichromophore may be loosely coupled or strongly coupled in aggregates. In the case of strongly coupled aggregates, it is preferable to have aggregates that give rise to enhanced emission intensity (more efficient emission) relative to the individual chromophores. These processes have been referred to as aggregation induced emission or the formation of J-aggregates. Aggregation induced emission refers to the aggregation rigidifying the molecules to prevent dissipation of the excited state energy through conformational dynamical processes. J-aggregates are produced when the electronic coupling increases the rate of the emission such that it competes more effectivity with other non-radiative processes.

In certain embodiments, the sensing material (e.g., the coupled-multichromophore) is configured to absorb a fluoroalkyl substance. In some embodiments, for example, the coupled-multichromophore is at least partially fluorinated such that the fluorinated domain of the coupled-multichromophore advantageously partitions fluoroalkyl substances into the sensing material. In some embodiments, a fluoroalkyl substance diffuses into the sensing material comprising an at least partially fluorinated coupled-multichromophore upon exposing the sensing material to the fluoroalkyl substance.

In some embodiments, the coupled-multichromophore comprises at least one fluoroalkyl group. In certain embodiments wherein the coupled-multichromophore comprises a conjugated polymer, the at least one fluoroalkyl group advantageously prevents close stacking between portions of the conjugated polymer. The at least one fluoroalkyl group of the coupled-multichromophore may comprise fluorine in any of a variety of suitable amounts. In some embodiments, for example, the at least one fluoroalkyl group of the coupled-multichromophore comprises fluorine in an amount greater than or equal to 25 weight percent (wt. %), greater than or equal to 30 wt. %, greater than or equal to 35 wt. %, greater than or equal to 40 wt. %, greater than or equal to 45 wt. %, greater than or equal to 50 wt. %, greater than or equal to 55 wt. %, greater than or equal to 60 wt. %, greater than or equal to 65 wt. %, or greater than or equal to 70 wt. % versus a total weight of the at least one fluoroalkyl group of the coupled-multichromophore. In certain embodiments, the at least one fluoroalkyl group of the coupled-multichromophore comprises fluorine in an amount less than or equal to 75 wt. %, less than or equal to 70 wt. %, less than or equal to 65 wt. %, less than or equal to 60 wt. %, less than or equal to 55 wt. %, less than or equal to 50 wt. %, less than or equal to 45 wt. %, less than or equal to 40 wt. %, less than or equal to 35 wt. %, or less than or equal to 30 wt. % versus a total weight of the at least one fluoroalkyl group of the coupled-multichromophore. Combinations of the above recited ranges are possible (e.g., the at least one fluoroalkyl group of the coupled-multichromophore comprises fluorine in an amount greater than or equal to 25 wt. % and less than or equal to 75 wt. % versus a total weight of the at least one fluoroalkyl group of the coupled-multichromophore, the at least one fluoroalkyl group of the coupled-multichromophore comprises fluorine in an amount greater than or equal to 50 wt. % and less than or equal to 55 wt. % versus a total weight of the at least one fluoroalkyl group of the coupled-multichromophore). Other ranges are also possible.

The coupled-multichromophore may comprise fluorine in any of a variety of suitable amounts. In some embodiments, for example, the coupled-multichromophore comprises fluorine in an amount greater than or equal to 25 weight percent (wt. %), greater than or equal to 30 wt. %, greater than or equal to 35 wt. %, greater than or equal to 40 wt. %, greater than or equal to 45 wt. %, greater than or equal to 50 wt. %, greater than or equal to 55 wt. %, greater than or equal to 60 wt. %, greater than or equal to 65 wt. %, or greater than or equal to 70 wt. % versus a total weight of the coupled-multichromophore. In certain embodiments, the coupled-multichromophore comprises fluorine in an amount less than or equal to 75 wt. %, less than or equal to 70 wt. %, less than or equal to 65 wt. %, less than or equal to 60 wt. %, less than or equal to 55 wt. %, less than or equal to 50 wt. %, less than or equal to 45 wt. %, less than or equal to 40 wt. %, less than or equal to 35 wt. %, or less than or equal to 30 wt. % versus a total weight of the coupled-multichromophore. Combinations of the above recited ranges are possible (e.g., the coupled-multichromophore comprises fluorine in an amount greater than or equal to 25 wt. % and less than or equal to 75 wt. % versus a total weight of the coupled-multichromophore, the coupled-multichromophore comprises fluorine in an amount greater than or equal to 50 wt. % and less than or equal to 55 wt. % versus a total weight of the coupled-multichromophore). Other ranges are also possible.

In certain embodiments, the coupled-multichromophore comprises a moiety that is capable of being protonated by the fluoroalkyl substance. In some embodiments wherein the coupled-multichromophore comprises a moiety that is capable of being protonated by the fluoroalkyl substance, one or more individual sites of the coupled-multichromophore comprise the moiety that is capable of being protonated by the fluoroalkyl substance and/or a portion of the moiety that is capable of being protonated by the fluoroalkyl substance. In some embodiments, for example, the coupled-multichromophore is capable of energy transport and/or diffusion between the moiety that is capable of being protonated by the fluoroalkyl substance and one or more other individual sites of the coupled-multichromophore (e.g., one or more repeating units of a polymer).

According to some embodiments, the moiety that is capable of being protonated by the fluoroalkyl substance is a Brønsted base. As used herein, the term “Brønsted base” is given its ordinary meaning in the art and refers to a species that is capable of accepting a proton (H⁺). In some embodiments, for example, the coupled-multichromophore comprises a nitrogen (N)-containing moiety that is capable of being protonated by the fluoroalkyl substance. The coupled-multichromophore may comprise any of a variety of suitable N-containing moieties. In some embodiments, for example, the coupled-multichromophore comprises a pyridine-containing moiety. In certain embodiments, the coupled-multichromophore comprises a pyridine- and a thiophene-containing moiety (e.g., a thiophene-functionalized pyridine-containing moiety). Without wishing to be bound by theory, a coupled-multichromophore comprising a pyridine- and a thiophene-containing moiety may advantageously display larger changes (e.g., shifts) in electromagnetic radiation (e.g., light) emission (as compared to, for example, a coupled-multichromophore comprising a pyridine-containing moiety) due to the π-electron delocalizing character of thiophene.

According to some embodiments, the coupled-multichromophore comprises a pyridine-containing moiety having the structure:

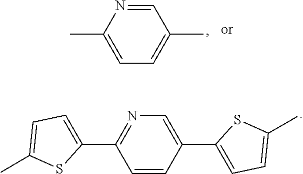

One of ordinary skill in the art will recognize that there are a number of other moieties that are capable of being protonated, including, for example, pyrazines, amines, imines, triazines, nitrogen-containing conjugated heterocycles, and the like.

According to certain embodiments, the interaction between the moiety that is capable of being protonated by the fluoroalkyl substance (e.g., the N-containing moiety such as a pyridine-containing moiety) and the fluoroalkyl substance is a bonding interaction. In some embodiments, for example, the interaction between the moiety that is capable of being protonated by the fluoroalkyl substance and the fluoroalkyl substance is an ionic bond resulting from a proton transfer reaction.

In some embodiments, at least one chromophore of the coupled-multichromophore displays a change in electromagnetic radiation (e.g., light) emission in response to protonation of the coupled-multichromophore by the fluoroalkyl substance. In certain embodiments, for example, the fluoroalkyl substance interacts with an individual site of the coupled-multichromophore (e.g., the moiety that is capable of being protonated by the fluoroalkyl substance) such that the fluoroalkyl substance protonates the individual site of the coupled-multichromophore, thereby resulting in the entire coupled-multichromophore (e.g., individual sites of the coupled-multichromophore that have not interacted with the fluoroalkyl substance and the individual site of the coupled-multichromophore that has interacted with the fluoroalkyl substance) displaying the change in electromagnetic radiation emission.

In certain embodiments, the change in electromagnetic radiation (e.g., light) emission of the at least one chromophore is a wavelength shift of an emission peak to a lower energy emission. In some embodiments, for example, the emission peak shifts by greater than or equal to 10 nm, greater than or equal to 50 nm, greater than or equal to 100 nm, or greater than or equal to 150 nm to a lower energy emission. In certain embodiments, the emission peak shifts by less than or equal to 200 nm, less than or equal to 150 nm, less than or equal to 100 nm, or less than or equal to 50 nm to a lower energy emission. Combinations of the above recited ranges are possible (e.g., the emission peak shifts by greater than or equal to 10 nm and less than or equal to 200 nm to a lower energy emission, the emission peak shifts by greater than or equal to 50 nm and less than or equal to 100 nm to a lower energy emission). Other ranges are also possible.

According to certain embodiments, the change in electromagnetic radiation (e.g., light) emission in response to protonation of the coupled-multichromophore by the fluoroalkyl substance may be an at least partially ratiometric change. In certain embodiments, for example, the change in electromagnetic radiation of the emission peak and the generation of a lower energy emission peak may be at least partially ratiometric.

In certain embodiments, the coupled-multichromophore comprises a dye. As used herein, the term “dye” is given its ordinary meaning in the art and refers to a molecule that is capable of absorbing electromagnetic radiation in the ultra-violet and/or visible range of the electromagnetic spectrum. In certain embodiments wherein the coupled-multichromophore comprises a dye, one or more individual sites of the coupled-multichromophore comprise the dye. In some embodiments, for example, the coupled-multichromophore is capable of energy transport and/or diffusion between the dye and one or more other individual sites of the coupled-multichromophore (e.g., one or more repeating units of a polymer).

The coupled-multichromophore may comprise any of a variety of suitable dyes. In certain embodiments, the dye is capable of accepting energy from one or more individual sites of the coupled-multichromophore. In some embodiments, for example, the dye comprises a small molecule and/or a polymer. In certain embodiments, the dye comprises squaraine, oxazine, perylene bisimide, coumarin, cyanine, conjugates thereof, and/or combinations thereof. Other dyes are also possible.

According to some embodiments, the dye is at least partially fluorinated. In certain embodiments, the fluorinated domain of the dye advantageously partitions fluoroalkyl substances into the sensing material. For example, in some embodiments, a fluoroalkyl substance diffuses into the coupled-multichromophore comprising an at least partially fluorinated dye upon exposing the coupled-multichromophore to a fluoroalkyl substance.

In some embodiments, the dye comprises at least one fluoroalkyl group. The at least one fluoroalkyl group of the dye may comprise fluorine in any of a variety of suitable amounts. In some embodiments, for example, the at least one fluoroalkyl group of the dye comprises fluorine in an amount greater than or equal to 25 wt. %, greater than or equal to 30 wt. %, greater than or equal to 35 wt. %, greater than or equal to 40 wt. %, greater than or equal to 45 wt. %, greater than or equal to 50 wt. %, greater than or equal to 55 wt. %, greater than or equal to 60 wt. %, greater than or equal to 65 wt. %, or greater than or equal to 70 wt. % versus a total weight of the at least one fluoroalkyl group of the dye. In certain embodiments, the at least one fluoroalkyl group of the dye comprises fluorine in an amount less than or equal to 75 wt. %, less than or equal to 70 wt. %, less than or equal to 65 wt. %, less than or equal to 60 wt. %, less than or equal to 55 wt. %, less than or equal to 50 wt. %, less than or equal to 45 wt. %, less than or equal to 40 wt. %, less than or equal to 35 wt. %, or less than or equal to 30 wt. % versus a total weight of the at least one fluoroalkyl group of the dye. Combinations of the above recited ranges are possible (e.g., the at least one fluoroalkyl group of the dye comprises fluorine in an amount greater than or equal to 25 wt. % and less than or equal to 75 wt. % versus a total weight of the at least one fluoroalkyl group of the dye, the at least one fluoroalkyl group of the dye comprises fluorine in an amount greater than or equal to 50 wt. % and less than or equal to 55 wt. % versus a total weight of the at least one fluoroalkyl group of the dye). Other ranges are also possible.

The dye may comprise fluorine in any of a variety of suitable amounts. In some embodiments, for example, the dye comprises fluorine in an amount greater than or equal to 25 wt. %, greater than or equal to 30 wt. %, greater than or equal to 35 wt. %, greater than or equal to 40 wt. %, greater than or equal to 45 wt. %, greater than or equal to 50 wt. %, greater than or equal to 55 wt. %, greater than or equal to 60 wt. %, greater than or equal to 65 wt. %, or greater than or equal to 70 wt. % versus a total weight of the dye. In certain embodiments, the dye comprises fluorine in an amount less than or equal to 75 wt. %, less than or equal to 70 wt. %, less than or equal to 65 wt. %, less than or equal to 60 wt. %, less than or equal to 55 wt. %, less than or equal to 50 wt. %, less than or equal to 45 wt. %, less than or equal to 40 wt. %, less than or equal to 35 wt. %, or less than or equal to 30 wt. % versus a total weight of the dye. Combinations of the above recited ranges are possible (e.g., the dye comprises fluorine in an amount greater than or equal to 25 wt. % and less than or equal to 75 wt. % versus a total weight of the dye, the dye comprises fluorine in an amount greater than or equal to 50 wt. % and less than or equal to 55 wt. % versus a total weight of the dye). Other ranges are also possible.

The coupled-multichromophore may comprise the dye in any of a variety of suitable amounts. In some embodiments, for example, the coupled-multichromophore comprises the dye in an amount greater than or equal to 0.1 wt. %, greater than or equal to 0.2 wt. %, greater than or equal to 0.3 wt. %, greater than or equal to 0.4 wt. %, greater than or equal to 1 wt. %, greater than or equal to 2 wt. %, greater than or equal to 3 wt. %, or greater than or equal to 4 wt. % versus a total weight of the coupled-multichromophore. In some embodiments, the coupled-multichromophore comprises the dye in an amount less than or equal to 5 wt. %, less than or equal to 4 wt. %, less than or equal to 3 wt. %, less than or equal to 2 wt. %, less than or equal to 1 wt. %, less than or equal to 0.5 wt. %, less than or equal to 0.4 wt. %, less than or equal to 0.3 wt. %, or less than or equal to 0.2 wt. % versus a total weight of the coupled-multichromophore. Combinations of the above recited ranges are possible (e.g., the coupled-multichromophore comprises the dye in an amount greater than or equal to 0.1 wt. % and less than or equal to 5 wt. % versus a total weight of the coupled-multichromophore, the coupled-multichromophore comprises the dye in an amount greater than or equal to 0.3 wt. % and less than or equal to 0.5 wt. % versus a total weight of the coupled-multichromophore). Other ranges are also possible.

According to some embodiments, the coupled-multichromophore comprises at least two chromophores that display a ratiometric change in electromagnetic radiation (e.g., light) emission in response to a presence of a fluoroalkyl substance. As used herein, the term “ratiometric” refers to the use of a ratio of intensities of two or more emission frequencies to provide information. In some embodiments, the ratio provides greater accuracy in the detection of an analyte than the individual emission intensities can provide as a singular signal. In certain embodiments, the ratio can provide information about the amount or concentration of an analyte.

In certain embodiments, the fluoroalkyl substance may interact with the coupled-multichromophore and change an organization of at least a portion of the coupled-multichromophore. In some embodiments, for example, the fluoroalkyl substance displaces at least a portion of the dye from the coupled-multichromophore and triggers an electron transfer interruption between one or more individual sites of the coupled-multichromophore and the dye, resulting in the at least two chromophores of the coupled-multichromophore displaying the ratiometric change in electromagnetic radiation emission. In some embodiments, the displacement is small and the dye is not physically removed from a matrix containing the coupled-multichromophore, but is displaced such that the orbital overlap between the dye and a chromophore of the coupled-multichromophore is reduced.

According to some embodiments, a first chromophore of the at least two chromophores may be capable of donating energy to a second chromophore of the at least two chromophores. Without wishing to be bound by theory, in certain embodiments wherein the coupled-multichromophore comprises a polymer and a dye, the polymer may act as a light-harvesting unit (e.g., a donor), which supplies energy to the dye (e.g., an acceptor) and amplifies a light emission from the dye. In certain embodiments, the polymer and the dye may have no or negligible spectral overlap.

In some embodiments, a first chromophore of the at least two chromophores displays an increase in electromagnetic radiation (e.g., light) emission in response to the presence of the fluoroalkyl substance. In certain embodiments wherein the coupled-multichromophore comprises a polymer and a dye, for example, a first chromophore corresponding to the polymer (e.g., corresponding to one or more repeating units of the polymer) displays an increase in electromagnetic radiation emission in response to the fluoroalkyl substance interacting with the coupled-multichromophore and changing an organization of at least a portion of the coupled-multichromophore. In some embodiments, for example, the fluoroalkyl substance displaces at least a portion of the dye from the coupled-multichromophore, which triggers an electron transfer interruption between the polymer (e.g., one or more repeating units of the polymer) and the dye.

The lifetime of the increase in electromagnetic radiation (e.g., light) emission of the first chromophore may be any of a variety of suitable values. In some embodiments, for example, the lifetime of the increase in electromagnetic radiation emission of the first chromophore is greater than or equal to 1 microsecond, greater than or equal to 2 microseconds, greater than or equal to 5 microseconds, greater than or equal to 10 microseconds, greater than or equal to 20 microseconds, greater than or equal to 50 microseconds, greater than or equal to 100 microseconds, or greater than or equal to 500 microseconds. In certain embodiments, the lifetime of the increase in electromagnetic radiation emission of the first chromophore is less than or equal to 1 millisecond, less than or equal to 500 microseconds, less than or equal to 100 microseconds, less than or equal to 50 microseconds, less than or equal to 20 microseconds, less than or equal to 10 microseconds, less than or equal to 5 microseconds, or less than or equal to 2 microseconds. Combinations of the above recited ranges are possible (e.g., the lifetime of the increase in electromagnetic radiation emission of the first chromophore is greater than or equal to 1 microsecond and less than or equal to 1 millisecond, the lifetime of the increase in electromagnetic radiation emission of the first chromophore is greater than or equal to 50 microseconds and less than or equal to 200 microseconds). Other ranges are also possible, including those over 1 millisecond, are also possible.

According to certain embodiments, a second chromophore of the at least two chromophores displays a decrease in electromagnetic radiation (e.g., light) emission in response to the presence of the fluoroalkyl substance. In certain embodiments wherein the coupled-multichromophore comprises a polymer and a dye, for example, a second chromophore corresponding to the dye displays a decrease in electromagnetic radiation emission in response to the fluoroalkyl substance interacting with at least a portion of the coupled-multichromophore and changing an organization of the coupled-multichromophore. In some embodiments, for example, the fluoroalkyl substance displaces at least a portion of the dye from the coupled-multichromophore, which triggers the electron transfer interruption between the polymer (e.g., one or more repeating units of the polymer) and the dye.

The lifetime of the decrease in electromagnetic radiation (e.g., light) emission of the second chromophore may be any of a variety of suitable values. In some embodiments, for example, the lifetime of the decrease in electromagnetic radiation emission of the second chromophore is greater than or equal to 1 nanosecond, greater than or equal to 2 nanoseconds, greater than or equal to 5 nanoseconds, greater than or equal to 10 nanoseconds, greater than or equal to 20 nanoseconds, greater than or equal to 50 nanoseconds, greater than or equal to 100 nanoseconds, or greater than or equal to 500 nanoseconds. In certain embodiments, the lifetime of the decrease in electromagnetic radiation emission of the second chromophore is less than or equal to 1 microsecond, less than or equal to 500 nanoseconds, less than or equal to 100 nanoseconds, less than or equal to 50 nanoseconds, less than or equal to 20 nanoseconds, less than or equal to 10 nanoseconds, less than or equal to 5 nanoseconds, or less than or equal to 2 nanoseconds. Combinations of the above recited ranges are possible (e.g., the lifetime of the decrease in electromagnetic radiation emission of the second chromophore is greater than or equal to 1 nanosecond and less than or equal to 1 millisecond, the lifetime of the decrease in electromagnetic radiation emission of the second chromophore is greater than or equal to 50 nanoseconds and less than or equal to 200 nanoseconds). Other ranges are also possible, including those over 1 millisecond, are also possible.

The coupled-multichromophore may have any of a variety of suitable weight-average molecular weights (Mw). In certain embodiments, for example, the coupled-multichromophore is a molecular and/or polymeric assembly having a weight-average molecular weight greater than or equal to 500 g/mol, greater than or equal to 1,000 g/mol, greater than or equal to 5,000 g/mol, greater than or equal to 10,000 g/mol, greater than or equal to 50,000 g/mol, greater than or equal to 100,000 g/mol, greater than or equal to 500,000 g/mol, greater than or equal to 1,000,000 g/mol, or greater than or equal to 1,500,000 g/mol. In some embodiments, the coupled-multichromophore is a molecular and/or polymeric assembly having a weight-average molecular weight less than or equal to 2,000,000 g/mol, less than or equal to 1,500,000 g/mol, less than or equal to 1,000,000 g/mol, less than or equal to 500,000 g/mol, less than or equal to 100,000 g/mol, less than or equal to 50,000 g/mol, less than or equal to 10,000 g/mol, less than or equal to 5,000 g/mol, or less than or equal to 1,000 g/mol. Combinations of the above recited ranges are possible (e.g., the coupled-multichromophore has a weight-average molecular weight greater than or equal to 500 g/mol and less than or equal to 2,000,000 g/mol, the coupled-multichromophore has a weight-average molecular weight greater than or equal to 50,000 g/mol and less than or equal to 100,000 g/mol). Other ranges are also possible. The weight-average molecular weight of the coupled-multichromophore may be determined by gel permeation chromatography.

The coupled-multichromophore may have any of a variety of suitable number-average molecular weights (Mn). In certain embodiments, for example, the coupled-multichromophore has a number-average molecular weight greater than or equal to 500 g/mol, greater than or equal to 1,000 g/mol, greater than or equal to 5,000 g/mol, greater than or equal to 10,000 g/mol, greater than or equal to 50,000 g/mol, greater than or equal to 100,000 g/mol, or greater than or equal to 500,000 g/mol. In some embodiments, the coupled-multichromophore has a number-average molecular weight less than or equal to 1,000,000 g/mol, less than or equal to 500,000 g/mol, less than or equal to 100,000 g/mol, less than or equal to 50,000 g/mol, less than or equal to 10,000 g/mol, less than or equal to 5,000 g/mol, or less than or equal to 1,000 g/mol. Combinations of the above recited ranges are possible (e.g., the coupled-multichromophore has a number-average molecular weight greater than or equal to 500 g/mol and less than or equal to 1,000,000 g/mol, the coupled-multichromophore has a number-average molecular weight greater than or equal to 50,000 g/mol and less than or equal to 100,000 g/mol). Other ranges are also possible. The number-average molecular weight of the coupled-multichromophore may be determined as by gel permeation chromatography.

The coupled-multichromophore may have any of a variety of suitable polydispersity indices. In certain embodiments, for example, the coupled-multichromophore has a polydispersity index greater than or equal to 1, greater than or equal to 1.5, greater than or equal to 2, or greater than or equal to 2.5. In some embodiments, the coupled-multichromophore has polydispersity index less than or equal to 3, less than or equal to 2.5, less than or equal to 2, or less than or equal to 1.5. Combinations of the above recited ranges are possible (e.g., the coupled-multichromophore has a polydispersity index greater than or equal to 1 and less than or equal to 3, the coupled-multichromophore has a polydispersity index greater than or equal to 2 and less than or equal to 2.5). Other ranges are also possible. The polydispersity index of the coupled-multichromophore may be determined by dividing the weight-average molecular weight of the coupled-multichromophore by the number-average molecular weight of the coupled-multichromophore.

The coupled-multichromophore may have any of a variety of suitable quantum yields. As used herein, the term “quantum yield” is given its ordinary meaning in the art and refers to a ratio of the number of photons emitted by the coupled-multichromophore to the number of photons absorbed by the coupled-multichromophore. The percent quantum yield of the coupled-multichromophore may be determined according to equation 1 (eq. 1) shown below.

$$Φ = \frac{number of photons emitted}{number of photons absorbed} \times 100 \qquad ( eq . 1 )$$

In certain embodiments, the quantum yield of the coupled-multichromophore is advantageously high such that the coupled-multichromophore has a long-excited state lifetime and a low non-radiative rate. According to certain embodiments, the coupled-multichromophore has a quantum yield greater than or equal to 30%, greater than or equal to 35%, greater than or equal to 40%, greater than or equal to 45%, greater than or equal to 50%, greater than or equal to 55%, greater than or equal to 60%, greater than or equal to 65% or greater than or equal to 70%. In some embodiments, the coupled-multichromophore has a quantum yield less than or equal to 75%, less than or equal to 70%, less than or equal to 65%, less than or equal to 60%, less than or equal to 55%, less than or equal to 50%, less than or equal to 45%, less than or equal to 40%, or less than or equal to 35%. Combinations of the above recited ranges are possible (e.g., the coupled-multichromophore has a quantum yield greater than or equal to 30% and less than or equal to 75%, the coupled-multichromophore has a quantum yield greater than or equal to 50% and less than or equal to 55%). Other ranges are also possible.

According to certain embodiments, the quantum yield of the coupled-multichromophore may change upon exposure to a fluoroalkyl substance. In some embodiments, for example, the quantum yield of the coupled-multichromophore decreases upon exposure to the fluoroalkyl substance. In other embodiments, the quantum yield of the coupled-multichromophore increases upon exposure to the fluoroalkyl substance.

The coupled-multichromophore may be synthesized by methods known to a person or ordinary skill in the art. In certain embodiments, for example, the coupled-multichromophore may be synthesized by one or more polymerization reactions (e.g., Suzuki polymerization, Sonogashira polymerization, free radical polymerization, cationic polymerization, metal catalyzed polymerization, ring opening polymerization, and/or condensation polymerization) of one or more monomers.

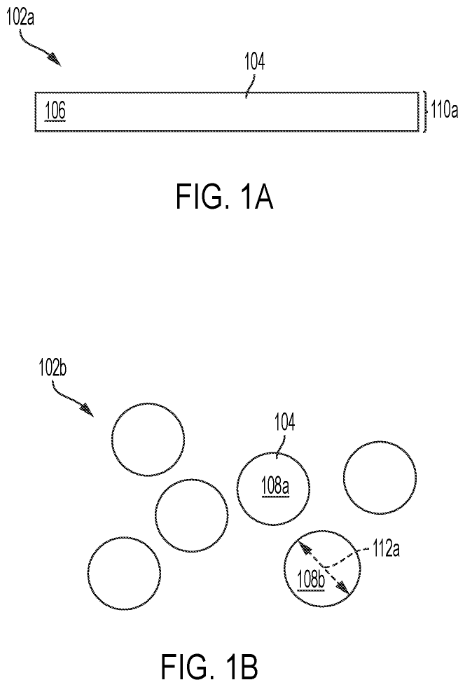

The sensing material may have any of a variety of suitable forms. In some embodiments, for example, the sensing material comprises a film (e.g., a thin film) comprising the coupled-multichromophore. FIG. 1A shows, according to certain embodiments, a cross-sectional schematic diagram of sensing material 102a comprising film 106 comprising coupled-multichromophore 104.

The film may have any of a variety of suitable thicknesses. Referring to FIG. 1A, for example, film 106 may have thickness 110a. In certain embodiments, the film has an average thickness greater than or equal to 0.1 nanometers, greater than or equal to 0.5 nanometers, greater than or equal to 1 nanometer, greater than or equal to 10 nanometers, greater than or equal to 100 nanometers, greater than or equal to 1 micrometer, greater than or equal to 10 micrometers, greater than or equal to 100 micrometers, greater than or equal to 1 millimeter, or greater than or equal to 1 centimeter. In some embodiments, the film has an average thickness less than or equal to 10 centimeters, less than or equal to 1 centimeter, less than or equal to 1 millimeter, less than or equal to 100 micrometers, less than or equal to 10 micrometers, less than or equal to 1 micrometer, less than or equal to 100 nanometers, less than or equal to 10 nanometers, less than or equal to 1 nanometer, or less than or equal to 0.5 nanometers. Combinations of the above recited ranges are possible (e.g., the film has an average thickness greater than or equal to 0.1 nanometers and less than or equal to 10 centimeters, the film has an average thickness greater than or equal to 100 nanometers and less than or equal to 1 micrometer). Other ranges are also possible. The average thickness of the film may be determined by electron microscopy techniques (e.g., scanning electron microscopy and/or transmission electron microscopy).

While film 106 comprising coupled-multichromophore 104 is depicted in FIG. 1A as a smooth layer of uniform thickness, those of ordinary skill in the art would understand that this is for illustration purposes only and the thickness of the film may have a particular roughness and/or may vary in thickness, in accordance with some embodiments. In some embodiments, for example, the film is of relatively uniform thickness (e.g., within +/−25% of the average thickness of the film, within +/−10% of the average thickness of the film, within +/−1% of the average thickness of the film).

The film may be formed by any of a variety of suitable methods. In some embodiments, for example, the film is formed by spin-casting, spin-coating, and/or dip-coating a solution of the coupled-multichromophore.

In certain embodiments, the sensing material comprises a plurality of particles comprising the coupled-multichromophore. FIG. 1B shows, according to certain embodiments, a cross-sectional schematic diagram of sensing material 102b comprising a plurality of particles 108 (e.g., particles 108a and 108b) comprising coupled-multichromophore 104. In some embodiments, the plurality of particles of the sensing material have a higher surface area as compared to, e.g., a film of the sensing material, which advantageously allows for increased detection of fluoroalkyl substances.

According to some embodiments, the particles may be magnetic such that the particle can be manipulated using a magnetic field.

Each particle of the plurality of particles may have any of a variety of suitable shapes. In some embodiments, for example, as shown in FIG. 1B, each particle of the plurality of particles has a spherical shape. In other embodiments, one or more particles of the plurality of particles have an angular shape, a cylindrical shape, a cubic shape, an elliptical shape, a fiber form, and/or the like.

According to some embodiments, the plurality of particles comprise a plurality of microparticles. The term “microparticle” is used herein in a manner consistent with its ordinary meaning in the art. Microparticles are particles having a maximum characteristic dimension (e.g., a maximum diameter) from 1 micrometer to 100 micrometers. The maximum characteristic dimension of a particle generally refers to the longest dimension from a first surface of the particle to a second surface of the particle that is substantially opposite the first surface. As one illustrative example, referring to FIG. 1B, particle 108b has maximum characteristic dimension 112a. According to some embodiments, the maximum characteristic dimension of the microparticle is from 1 micrometer to 10 micrometers, 10 micrometers to 20 micrometers, 20 micrometers to 30 micrometers, 30 micrometers to 50 micrometers, 50 micrometers to 70 micrometers, or 70 micrometers to 100 micrometers. Combinations of the above recited ranges are possible (e.g., 30 micrometers to 70 micrometers, or 20 micrometers to 100 micrometers). Other ranges are also possible. The maximum characteristic dimension of the microparticle may be determined by electron microscopy techniques (e.g., scanning electron microscopy and/or transmission electron microscopy).

In certain embodiments, the plurality of particles comprise a plurality of nanoparticles. The term “nanoparticle” is used herein in a manner consistent with its ordinary meaning in the art. Nanoparticles are particles having a maximum characteristic dimension from 1 nanometer to 1 micrometer. According to some embodiments, the maximum characteristic dimension of the nanoparticle is from 1 nanometer to 100 nanometers, 100 nanometers to 200 nanometers, 200 nanometers to 300 nanometers, 300 nanometers to 500 nanometers, 500 nanometers to 700 nanometers, or 700 nanometers to 1 micrometer. Combinations of the above recited ranges are possible (e.g., 300 nanometers to 700 nanometers, or 200 nanometers to 1 micrometer). Other ranges are also possible. The maximum characteristic dimension of the nanoparticle may be determined by electron microscopy techniques (e.g., scanning electron microscopy and/or transmission electron microscopy).

According to some embodiments, the plurality of particles comprises a combination of particles having different maximum characteristic dimensions. According to some embodiments, for example, the plurality of particles comprises at least one microparticle and at least one nanoparticle.

The plurality of particles may be formed by any of a variety of suitable methods. In some embodiments, for example, the plurality of particles is formed by adding a non-aqueous solution of the coupled-multichromophore to water under sonication, followed by evaporating the non-aqueous solvent.

In certain embodiments, the analyte described herein is a fluoroalkyl substance. As used herein, the term “fluoroalkyl substance” refers to a molecule comprising an alkyl group in which one or more hydrogens have been substituted with fluorine. The fluoroalkyl substance may be any of a variety of suitable fluoroalkyl substances. In certain embodiments, the fluoroalkyl substance is a per- or poly-fluoroalkyl substance (PFAS). In certain embodiments, the fluoroalkyl substance comprises 2-(N-methyl-perfluorooctane sulfonamido) acetic acid, perfluorobutane sulfonic acid, perfluorohexane sulfonic acid, perfluoroheptanoic acid, perfluorooctane sulfonic acid, perfluoromethylheptane sulfonic acid, perfluorooctanoic acid, perfluorononanoic acid, perfluorodecanoic acid, perfluoroundecanoic acid, perfluorododecanoic acid, conjugates thereof, and/or combinations thereof. Other fluoroalkyl substances are also possible.

According to some embodiments, the fluoroalkyl substance is a Brønsted acid. As used herein, the term “Brønsted acid” is given its ordinary meaning in the art and refers to a species that is capable of donating a proton (H⁺).

The sensing material may have any of a variety of suitable sensitivities. In certain embodiments, the sensing material is capable of detecting the presence of a fluoroalkyl substance at a parts per million (ppm) level. In some embodiments, for example, the sensing material is capable of detecting the presence of the fluoroalkyl substance at a sensitivity less than or equal to 1000 ppm, less than or equal to 100 ppm, or less than or equal to 10 ppm. In certain embodiments, the sensing material is capable of detecting the presence of the fluoroalkyl substance at a sensitivity greater than or equal to 1 ppm, greater than or equal to 10 ppm, or greater than or equal to 100 ppm. Combinations of the above recited ranges are possible (e.g., the sensing material is capable of detecting the presence of the fluoroalkyl substance at a sensitivity less than or equal to 1000 ppm and greater than or equal to 1 ppm, the sensing material is capable of detecting the presence of the fluoroalkyl substance at a sensitivity less than or equal to 100 ppm and greater than or equal to 10 ppm). Other ranges are also possible.

In some embodiments, the sensing material is capable of detecting the presence of the fluoroalkyl substance at a parts per billion (ppb) level. In some embodiments, for example, the sensing material is capable of detecting the presence of the fluoroalkyl substance at a sensitivity less than or equal to 1000 ppb, less than or equal to 900 ppb, less than or equal to 800 ppb, less than or equal to 700 ppb, less than or equal to 600 ppb, less than or equal to 500 ppb, less than or equal to 400 ppb, less than or equal to 300 ppb, less than or equal to 200 ppb, less than or equal to 100 ppb, less than or equal to 50 ppb, or less than or equal to 10 ppb. In certain embodiments, the sensing material is capable of detecting the presence of the fluoroalkyl substance at a sensitivity greater than or equal to 1 ppb, greater than or equal to 10 ppb, greater than or equal to 50 ppb, greater than or equal to 100 ppb, greater than or equal to 200 ppb, greater than or equal to 300 ppb, greater than or equal to 400 ppb, greater than or equal to 500 ppb, greater than or equal to 600 ppb, greater than or equal to 700 ppb, greater than or equal to 800 ppb, or greater than or equal to 900 ppb. Combinations of the above recited ranges are possible (e.g., the sensing material is capable of detecting the presence of the fluoroalkyl substance at a sensitivity less than or equal to 1000 ppb and greater than or equal to 1 ppb, the sensing material is capable of detecting the presence of the fluoroalkyl substance at a sensitivity less than or equal to 500 ppb and greater than or equal to 400 ppb). Other ranges are also possible.

According to certain embodiments, the sensing material is capable of detecting the presence of a fluoroalkyl substance at a parts per trillion (ppt) level. In some embodiments, for example, the sensing material is capable of detecting the presence of the fluoroalkyl substance at a sensitivity less than or equal to 1000 ppt, less than or equal to 900 ppt, less than or equal to 800 ppt, less than or equal to 700 ppt, less than or equal to 600 ppt, less than or equal to 500 ppt, less than or equal to 400 ppt, less than or equal to 300 ppt, less than or equal to 200 ppt, less than or equal to 100 ppt, less than or equal to 50 ppt, or less than or equal to 10 ppt. In certain embodiments, the sensing material is capable of detecting the presence of the fluoroalkyl substance at a sensitivity greater than or equal to 1 ppt, greater than or equal to 10 ppt, greater than or equal to 50 ppt, greater than or equal to 100 ppt, greater than or equal to 200 ppt, greater than or equal to 300 ppt, greater than or equal to 400 ppt, greater than or equal to 500 ppt, greater than or equal to 600 ppt, greater than or equal to 700 ppt, greater than or equal to 800 ppt, or greater than or equal to 900 ppt. Combinations of the above recited ranges are possible (e.g., the sensing material is capable of detecting the presence of the fluoroalkyl substance at a sensitivity less than or equal to 1000 ppt and greater than or equal to 1 ppt, the sensing material is capable of detecting the presence of the fluoroalkyl substance at a sensitivity less than or equal to 500 ppt and greater than or equal to 400 ppt). Other ranges are also possible.

According to certain embodiments, an article is described. In some embodiments, the article comprises a sensing material comprising a coupled-multichromophore, which is described in greater detail elsewhere herein.

According to some embodiments, the article is a film comprising the coupled-multichromophore, which is described in greater detail elsewhere herein with respect to FIG. 1A.

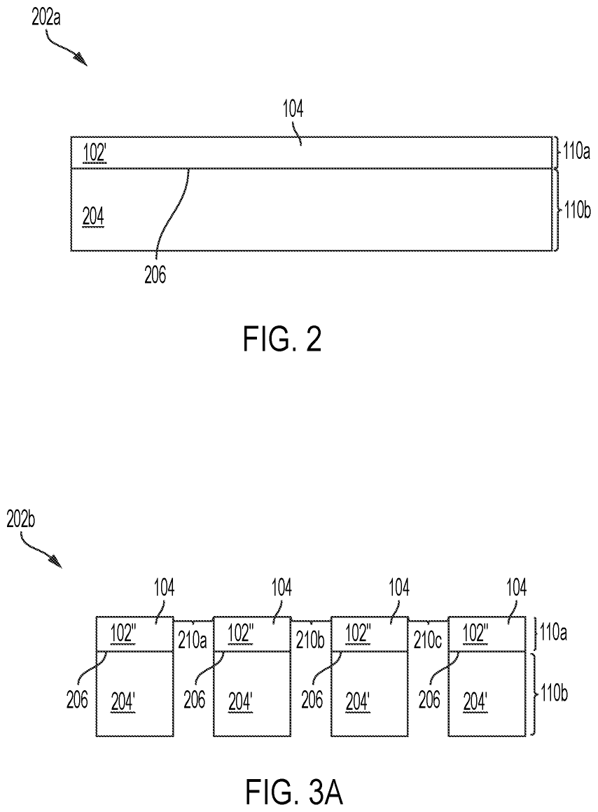

In certain embodiments, the article further comprises a substrate. In some embodiments, for example, the sensing material is disposed on at least a portion of the substrate. FIG. 2 shows, according to certain embodiments, a cross-sectional schematic diagram of article 202a comprising substrate 204 and sensing material 102′ comprising coupled-multichromophore 104 disposed on at least a portion of substrate 204. According to some embodiments, as shown in FIG. 2, sensing material 102′ is disposed on surface 206 of substrate 204 such that sensing material 102′ coats surface 206 of substrate 204. In certain embodiments, sensing material 102′ is chemically grafted to surface 206 of substrate 204.

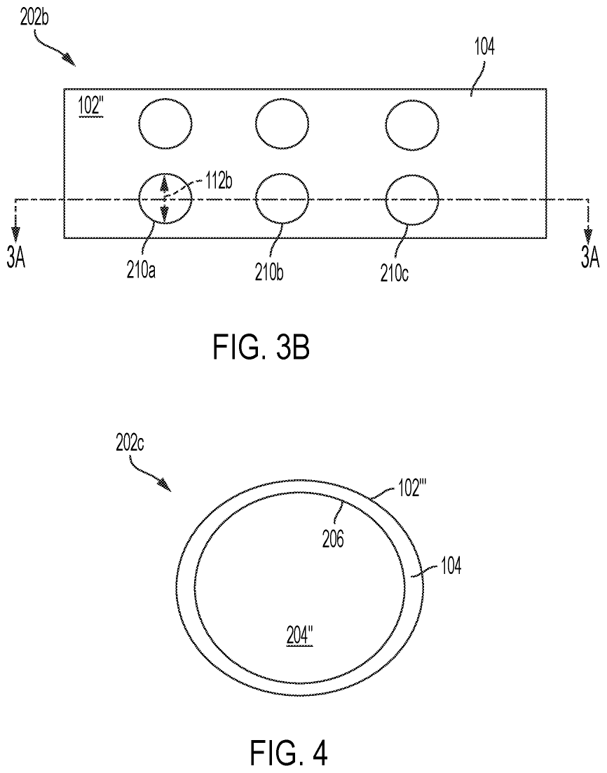

According to some embodiments, the substrate and/or the sensing material disposed on the surface of the substrate comprise a plurality of pores. FIG. 3A shows, according to certain embodiments, a cross-sectional schematic diagram of article 202b comprising substrate 204′ and sensing material 102″ comprising coupled-multichromophore 104, wherein substrate 204′ and sensing material 102″ comprise a plurality of pores 210 (e.g., pores 210a, 210b, and 210c). FIG. 3B shows, according to certain embodiments, a top-view schematic diagram of article 202b, wherein the cross-section shown in FIG. 3A is taken along dotted-line 3A.

In certain embodiments, as shown in FIG. 3A, sensing material 102″ is disposed on surface 206 of substrate 204′ such that sensing material 102″ coats surface 206 of substrate 204′. In certain embodiments, sensing material 102″ is chemically grafted to surface 206 of substrate 204′.

In certain embodiments wherein the substrate and/or the sensing material comprise a plurality pores, the article may be a filter and/or a membrane. Referring to FIGS. 3A-3B, for example, article 202b may be a filter and/or a membrane.

Each pore of the plurality of pores may have any of a variety of suitable shapes. In some embodiments, for example, as shown in FIG. 3B, each pore of the plurality of pores has a circular shape. In other embodiments, one or more pores of the plurality of pores have an oval shape, a square shape, a rectangular shape, a triangular shape, and/or the like.

The plurality of pores may have any of a variety of suitable maximum characteristic dimensions. The maximum characteristic dimension of a pore generally refers to the longest dimension from a first surface of the pore to a second surface of the pore that is substantially opposite the first surface. As one illustrative example, referring to FIG. 3B, pore 210a has a maximum characteristic dimension 112b. According to certain embodiments, each pore of the plurality of pores has a maximum characteristic dimension greater than or equal to 2 nanometers, greater than or equal to 10 nanometers, greater than or equal to 100 nanometers, greater than or equal to 500 nanometers, greater than or equal to 1 micrometer, greater than or equal to 10 micrometers, or greater than or equal to 50 micrometers. In some embodiments, each pore of the plurality of pores has a maximum characteristic dimension less than or equal to 100 micrometers, less than or equal to 50 micrometers, less than or equal to 10 micrometers, less than or equal to 1 micrometer, less than or equal to 500 nanometers, less than or equal to 100 nanometers, less than or equal to 100 nanometers, or less than or equal to 10 nanometers. Combinations of the above recited ranges are possible (e.g., each pore of the plurality of pores has a maximum characteristic dimension greater than or equal to 2 nanometers and less than or equal to 100 micrometers, each pore of the plurality of pores has a maximum characteristic dimension greater than or equal to 500 nanometers and less than or equal to 10 micrometers). Other ranges are also possible.

The substrate may have any of a variety of suitable thicknesses. Referring to FIGS. 2-3A, for example, substrate 204 (e.g., substrate 204 in FIG. 2 and substrate 204′ in FIG. 3A) has thickness 110b. In certain embodiments, the substrate has an average thickness greater than or equal to 0.1 micrometers, greater than or equal to 1 micrometer, greater than or equal to 10 micrometers, greater than or equal to 100 micrometers, greater than or equal to 1 millimeter, greater than or equal 1 centimeter, or greater than or equal to 10 centimeters. In some embodiments, the substrate has an average thickness less than or equal to 1 meter, less than or equal to 10 centimeters, less than or equal to 1 centimeter, less than or equal to 1 millimeter, less than or equal to 100 micrometers, less than or equal to 10 micrometers, or less than or equal to 1 micrometer. Combinations of the above recited ranges are possible (e.g., the substrate has an average thickness greater than or equal to 0.1 micrometers and less than or equal to 1 meter, the substrate has an average thickness greater than or equal to 100 micrometers and less than or equal to 1 millimeter). Other ranges are also possible.

While substrate 204 (e.g., substrate 204 in FIG. 2 and substrate 204′ in FIG. 3A) is depicted in FIGS. 2-3A as a smooth layer of uniform thickness, those of ordinary skill in the art would understand that this is for illustration purposes only and the thickness of the substrate may have a particular roughness and/or may vary in thickness, in accordance with some embodiments. In some embodiments, for example, the substrate is of relatively uniform thickness (e.g., within +/−25% of the average thickness of the substrate, within +/−10% of the average thickness of the substrate, within +/−1% of the average thickness of the substrate).

The sensing material disposed on the surface of the substrate may have any of a variety of suitable thicknesses. Referring to FIGS. 2-3A, for example, sensing material 102 (e.g., sensing material 102′ in FIG. 2 and sensing material 102″ in FIG. 3A) disposed on surface 206 of substrate 204 (e.g., substrate 204 in FIG. 2 and substrate 204′ in FIG. 3A) may have thickness 110a. In certain embodiments, the sensing material disposed on the surface of the substrate has an average thickness as described herein in greater detail with respect to the film of the sensing material comprising the coupled-multichromophore as shown in FIG. 1A. In some embodiments, for example, the sensing material disposed on the surface of the substrate has an average thickness greater than or equal to 0.1 nanometers and less than or equal to 10 centimeters.

While sensing material 102 (e.g., sensing material 102′ in FIG. 2 and sensing material 102″ in FIG. 3A) disposed on surface 206 of substrate 204 (e.g., substrate 204 in FIG. 2 and substrate 204′ in FIG. 3A) is depicted in FIGS. 2-3A as a smooth layer of uniform thickness, those of ordinary skill in the art would understand that this is for illustration purposes only and the thickness of the sensing material disposed on the surface of the substrate may have a particular roughness and/or may vary in thickness, in accordance with some embodiments. In some embodiments, the sensing material disposed on the surface of the substrate is of relatively uniform thickness (e.g., within +/−25% of the average thickness of the sensing material, within +/−10% of the average thickness of the sensing material, within +/−1% of the average thickness of the sensing material) over at least a substantial portion of the surface of the substrate (e.g., greater than or equal to 75% of the surface area of the surface of the substrate on which the sensing material is disposed, greater than or equal to 90% of the surface area of the surface of the substrate on which the sensing material is disposed, greater than or equal to 99% of the surface area of the surface of the substrate) on which the sensing material is disposed.

The substrate may comprise any of a variety of suitable materials. In some embodiments, for example, the substrate comprises glass, a ceramic, a polymer, cellulose, nitrocellulose, a metal, a metal oxide, concrete, a zeolite, a mesoporous silicate, an anodized alumina filter, and/or combinations thereof. In certain embodiments, the substrate comprises a fiber optic, an optical cavity, an optical waveguide, and/or a grating. In certain embodiments, the substrate comprises cloth, a woven material, and/or a nanofiber matt. In some embodiments, the substrate comprises a filter material comprising fibers, one or more polymers, glass, a ceramic, a metal oxide, and/or combinations thereof.

In certain embodiments, the substrate comprises a particle. FIG. 4 shows, according to certain embodiments, a cross-sectional schematic diagram of article 202c comprising substrate 204″ and sensing material 102′″ comprising coupled-multichromophore 104 disposed on at least a portion of substrate 204″, wherein substrate 204″ comprises a particle.

In certain embodiments, as shown in FIG. 4, sensing material 102′″ is disposed on surface 206 of substrate 204″ such that sensing material 102′″ coats surface 206 of substrate 204″. In certain embodiments, sensing material 102′″ is chemically grafted to surface 206 of substrate 204″.

The particle may have any of a variety of suitable shapes. In some embodiments, for example, as shown in FIG. 4, the particle has a spherical shape. In other embodiments, the particle has an angular shape, a cylindrical shape, a cubic shape, an elliptical shape, a fiber form, and/or the like.

The particle may have any of a variety of suitable sizes. According to certain embodiments, the particle is a microparticle (e.g., having a maximum characteristic dimension from 1 micrometer to 100 micrometers). In certain embodiments, the particle is a nanoparticle (e.g., having a maximum characteristic dimension from 1 nanometer to 1 micrometer).

The particle may comprise any of a variety of suitable materials. In accordance with certain embodiments, the particle is magnetic such that the particle can be manipulated using a magnetic field. In certain embodiments, for example, a magnet can be used to move, collect, organize, and/or localize the one or more particles. In some embodiments, a magnet can be used to remove one or more magnetic particles from a solution to which the magnetic particles have been added (e.g., to detect a presence of a fluoroalkyl substance in the solution, as described herein in greater detail). In some embodiments, the particle comprises a metal. In certain embodiments, for example, the particle comprises iron (Fe), nickel (Ni), cobalt (Co), and/or combinations thereof. In some embodiments, the particle comprises a zeolite. In certain embodiments, the particle comprises a polymer. Other materials and/or combinations of the materials mentioned above are also possible.

According to certain embodiments, a method of detecting a fluoroalkyl substance is described. In some embodiments, the method comprises exposing a coupled-multichromophore to a solution comprising the fluoroalkyl substance. According to some embodiments, the exposing comprises exposing the coupled-multichromophore to the fluoroalkyl substance such that the coupled-multichromophore absorbs the fluoroalkyl substance.

The solution may be any of a variety of suitable solutions. In some embodiments, for example, the solution is an aqueous solution. Any of a variety of suitable aqueous solutions may be used, including, but not limited to, deionized water, tap water, well water, wastewater, reservoir water, ocean water, sea water, pond water, lake water, river water, water from an industrial site, water from a semiconductor manufacturing site, water distributed to towns and/or cities, aqueous food solutions, and the like. In other embodiments, the solution is a non-aqueous solution. In certain embodiments, for example, the solution comprises an organic solvent.

The fluoroalkyl substance may by any of a variety of suitable fluoroalkyl substances, as described in greater detail elsewhere herein. In some embodiments, for example, the fluoroalkyl substance is a PFAS.

According to some embodiments, the solution comprises one or more non-target analytes, such as one or more metal ions, polymers, biopolymers, humic acids, and/or organic oils or surfactants. Other non-target analytes are also possible.

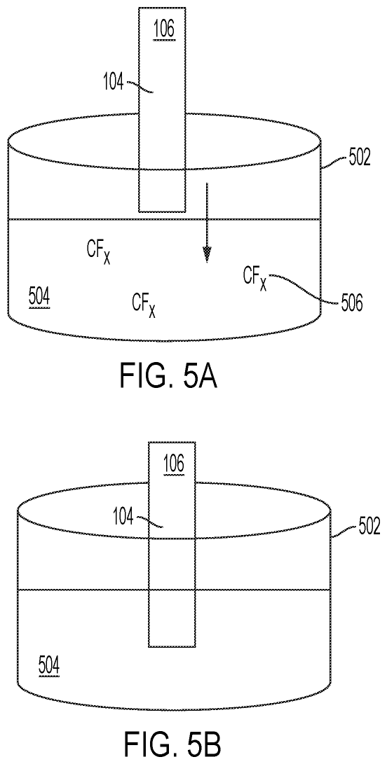

In certain embodiments, the exposing comprises exposing a film of the sensing material comprising the coupled-multichromophore to the solution comprising the fluoroalkyl substance. FIGS. 5A-5B show, according to certain embodiments, a schematic diagram representing a method comprising exposing film 106 of the sensing material comprising coupled-multichromophore 104 to solution 504 comprising fluoroalkyl substance 506, which is represented generally as “CFₓ” in the figures. According to certain embodiments, as shown in FIG. 5A, film 106 of the sensing material comprising coupled-multichromophore 104 is inserted into solution 504 comprising fluoroalkyl substance 506. In some embodiments, as shown in FIG. 5B, coupled-multichromophore 104 absorbs fluoroalkyl substance 506 after inserting film 106 of the sensing material comprising coupled-multichromophore 104 into solution 504 comprising fluoroalkyl substance 506. In some embodiments, for example, once solution 504 comprising fluoroalkyl substance 506 contacts a surface of film 106 of the sensing material comprising coupled-multichromophore 104, fluoroalkyl substance 506 diffuses into coupled-multichromophore 104. According to certain embodiments, as shown in FIGS. 5A-5B, solution 504 is contained within container 502.

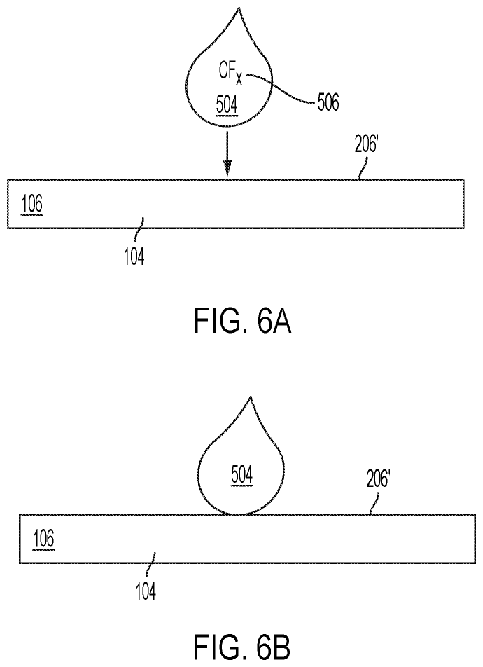

FIGS. 6A-6B show, according to certain embodiments, a schematic diagram representing another method comprising exposing film 106 of the sensing material comprising coupled-multichromophore 104 to solution 504 comprising fluoroalkyl substance 506. According to certain embodiments, as shown in FIG. 6A, solution 504 comprising fluoroalkyl substance 506 is added to surface 206′ of film 106 of the sensing material comprising coupled-multichromophore 104. In certain embodiments, for example, solution 504 comprising fluoroalkyl substance 506 may be poured onto, drop-casted onto, and/or flowed over surface 206′ of film 106 of the sensing material comprising coupled-multichromophore 104. In some embodiments, as shown in FIG. 6B, coupled-multichromophore 104 absorbs fluoroalkyl substance 506 after solution 504 comprising fluoroalkyl substance 506 is added to surface 206′ of film 106 of the sensing material comprising coupled-multichromophore 104. For example, in some embodiments, once solution 504 comprising fluoroalkyl substance 506 contacts surface 206′ of film 106 of the sensing material comprising coupled-multichromophore 104, fluoroalkyl substance 506 diffuses into coupled-multichromophore 104.

According to certain embodiments, the film of the sensing material comprising the coupled-multichromophore may be configured as a lateral flow assay architecture. In some such embodiments, upon exposing the film of the sensing material comprising the coupled-multichromophore to the solution comprising the fluoroalkyl substance, at least a portion of the solution comprising the fluoroalkyl substance may flow along a surface of the film via capillary action. Referring, for example, to FIGS. 6A-6B, upon exposing film 106 of the sensing material comprising coupled-multichromophore 104 to solution 504 comprising fluoroalkyl substance 506, at least a portion of solution 504 comprising fluoroalkyl substance 506 may flow along surface 206′ of film 106 via capillary action.

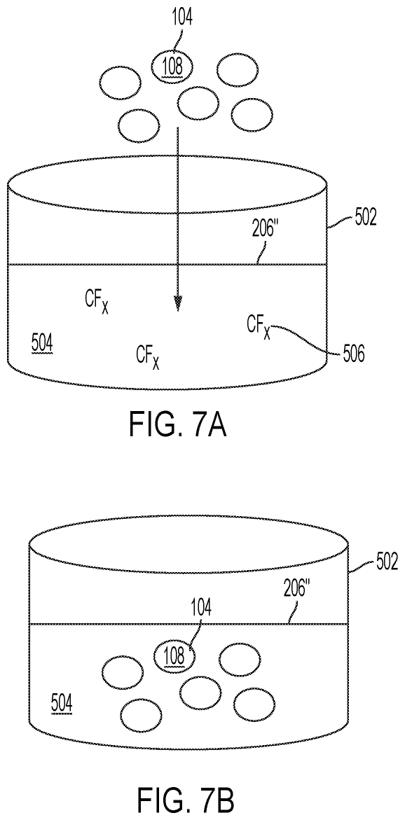

In some embodiments, the exposing comprises exposing a plurality of particles of the sensing material comprising the coupled-multichromophore to the solution comprising the fluoroalkyl substance. FIGS. 7A-7B show, according to certain embodiments, a schematic diagram representing a method comprising exposing plurality of particles 108 of the sensing material comprising coupled-multichromophore 104 to solution 504 comprising fluoroalkyl substance 506. According to certain embodiments, as shown in FIG. 7A, plurality of particles 108 of the sensing material comprising coupled-multichromophore 104 is added to solution 504 comprising fluoroalkyl substance 506. In certain embodiments, as shown in FIGS. 7A-7B, solution 504 is contained within container 502. According to some embodiments, one or more particles 108 have a density greater than a density of solution 504 such that one or more particles 108 sink in solution 504 (e.g., to a bottom of container 502). In certain embodiments, one or more particles 108 have a density less than the density of solution 504 such that one or more particles 108 float on surface 206″ of solution 504. According to certain embodiments in which the particles are magnetic, a magnetic may be used to collect, move, organize, and/or localize the magnetic particles, as explained herein in greater detail.

In some embodiments, as shown in FIG. 7B, coupled-multichromophore 104 absorbs fluoroalkyl substance 506 after adding plurality of particles 108 of the sensing material comprising coupled-multichromophore 104 to solution 504 comprising fluoroalkyl substance 506. For example, in certain embodiments, once solution 504 comprising fluoroalkyl substance 506 contacts a surface of plurality of particles 108 of the sensing material comprising coupled-multichromophore 104, fluoroalkyl substance 506 diffuses into coupled-multichromophore 104.

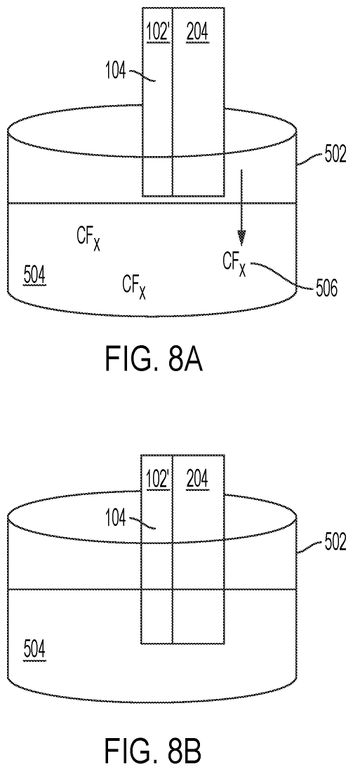

According to certain embodiments, the exposing comprises exposing a substrate and the sensing material comprising the coupled-multichromophore disposed on the substrate to the solution comprising the fluoroalkyl substance. FIGS. 8A-8B show, according to certain embodiments, a schematic diagram representing a method comprising exposing substrate 204 and sensing material 102′ comprising coupled-multichromophore 104 disposed on substrate 204 to solution 504 comprising fluoroalkyl substance 506. According to certain embodiments, as shown in FIG. 8A, substrate 204 and sensing material 102′ comprising coupled-multichromophore 104 disposed on substrate 204 is inserted into solution 504 comprising fluoroalkyl substance 506. In some embodiments, as shown in FIG. 8B, coupled-multichromophore 104 absorbs fluoroalkyl substance 506 after inserting substrate 204 and sensing material 102′ comprising coupled-multichromophore 104 disposed on substrate 204 into solution 504 comprising fluoroalkyl substance 506. For example, in some embodiments, once solution 504 comprising fluoroalkyl substance 506 contacts a surface of sensing material 102′ comprising coupled-multichromophore 104, fluoroalkyl substance 506 diffuses into coupled-multichromophore 104. In certain embodiments, as shown in FIGS. 8A-8B, solution 504 is contained within container 502.

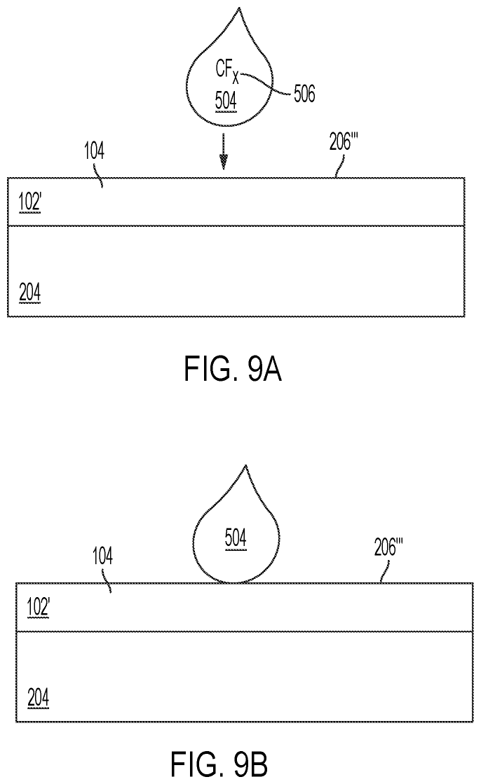

FIGS. 9A-9B show, according to certain embodiments, a schematic diagram representing another method comprising exposing substrate 204 and sensing material 102′ comprising coupled-multichromophore 104 disposed on substrate 204 to solution 504 comprising fluoroalkyl substance 506. In some embodiments, as shown in FIG. 9A, solution 504 comprising fluoroalkyl substance 506 is added to surface 206′″ of sensing material 102′ comprising coupled-multichromophore 104. In some embodiments, for example, solution 504 comprising fluoroalkyl substance 506 may be poured onto, drop-casted onto, and/or flowed over surface 206′″ of sensing material 102′ comprising coupled-multichromophore 104. In some embodiments, as shown in FIG. 9B, coupled-multichromophore 104 absorbs fluoroalkyl substance 506 after solution 504 comprising fluoroalkyl substance 506 is added to surface 206′″ of sensing material 102′ comprising coupled-multichromophore 104. In certain embodiments, for example, once solution 504 comprising fluoroalkyl substance 506 contacts surface 206′″ of sensing material 102′ coupled-multichromophore 104, fluoroalkyl substance 506 diffuses into coupled-multichromophore 104.

According to some embodiments, the substrate and the sensing material comprising the coupled-multichromophore disposed on the substrate may be configured as a lateral flow assay architecture. In some such embodiments, upon exposing the substrate and the sensing material comprising the coupled-multichromophore disposed on the substrate to the solution comprising the fluoroalkyl substance, at least a portion of the solution comprising the fluoroalkyl substance may flow along a surface of the sensing material via capillary action. Referring, for example, to FIGS. 9A-9B, upon exposing substrate 204 and sensing material 102′ comprising coupled-multichromophore 104 disposed on substrate 204 to solution 504 comprising fluoroalkyl substance 506, at a least a portion of solution 504 comprising fluoroalkyl substance 506 may flow along surface 206′″ of sensing material 102′ via capillary action.

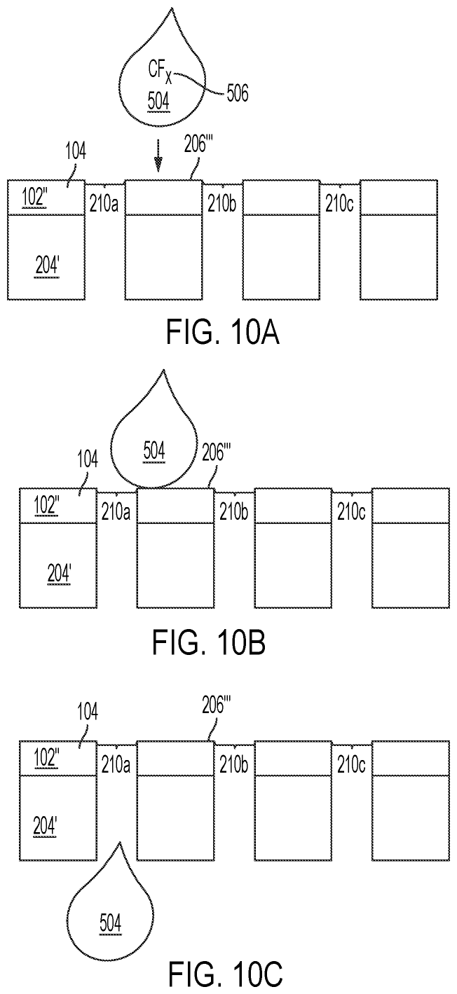

FIGS. 10A-10C show, according to certain embodiments, a schematic diagram representing a method comprising exposing substrate 204′ and sensing material 102″ comprising coupled-multichromophore 104 disposed on substrate 204′ to solution 504 comprising fluoroalkyl substance 506, wherein substrate 204′ and sensing material 102″ comprise a plurality of pores 210 (e.g., pores 210a, 210b, and 210c). In some embodiments, as shown in FIG. 10A, solution 504 comprising fluoroalkyl substance 506 is added to surface 206′″′ of sensing material 102″ comprising coupled-multichromophore 104. In certain embodiments, for example, solution 504 comprising fluoroalkyl substance 506 may be poured onto, drop-casted onto, and/or flowed over surface 206′″ of sensing material 102″ comprising coupled-multichromophore 104. In some embodiments, as shown in FIG. 10B, coupled-multichromophore 104 absorbs fluoroalkyl substance 506 after solution 504 comprising fluoroalkyl substance 506 is added to surface 206′″ of sensing material 102″ comprising coupled-multichromophore 104. For example, in some embodiments, once solution 504 comprising fluoroalkyl substance 506 contacts surface 206′″ of sensing material 102″ comprising coupled-multichromophore 104, fluoroalkyl substance 506 diffuses into coupled-multichromophore 104. According to certain embodiments, as shown in FIG. 10C, solution 504 may flow through one or more pores 210 (e.g., pores 210a, 210b, and/or 210c) after coupled-multichromophore 104 absorbs fluoroalkyl substance 506.

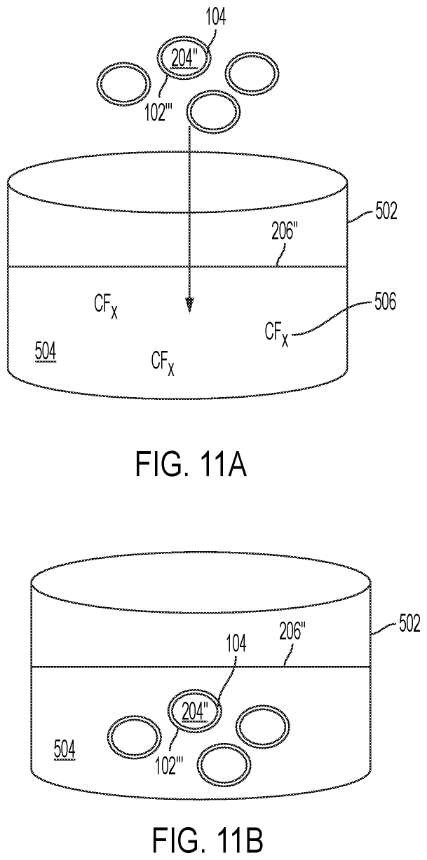

FIGS. 11A-11B show, according to certain embodiments, a schematic diagram representing a method comprising exposing substrate 204″ and sensing material 102′″ comprising coupled-multichromophore 104 disposed on substrate 204′ to solution 504 comprising fluoroalkyl substance 506, wherein substrate 204″ comprises a particle. In certain embodiments, as shown in FIG. 11A, plurality of substrates 204″ (e.g., particle substrates) and sensing material 102′″ comprising coupled-multichromophore 104 disposed on substrates 204″ is added to solution 504 comprising fluoroalkyl substance 506. In certain embodiments, as shown in FIG. 11A-11B, solution 504 is contained within container 502. In some embodiments, one or more substrates 204″ (e.g., particle substrates) and sensing material 102′″ comprising coupled-multichromophore 104 disposed on one or more substrates 204″ have a density greater than a density of solution 504 such that one or more substrates 204″ and sensing material 102′″ comprising coupled-multichromophore 104 disposed on one or more substrates 204″ sink in solution 504 (e.g., to a bottom of container 502). In some embodiments, one or more substrates 204″ (e.g., particle substrates) and sensing material 102′″ comprising coupled-multichromophore 104 disposed on one or more substrates 204″ have a density less than the density of solution 504 such that one or more substrates 204″ and sensing material 102′″ comprising coupled-multichromophore 104 disposed on one or more substrates 204″ float on surface 206″ of solution 504. According to certain embodiments in which the particles are magnetic, a magnetic may be used to collect, move, organize, and/or localize the magnetic particles, as explained herein in greater detail.

In some embodiments, as shown in FIG. 11B, coupled-multichromophore 104 absorbs fluoroalkyl substance 506 after adding plurality of substrates 204″ (e.g., particle substrates) and sensing material 102′″ comprising coupled-multichromophore 104 disposed on substrates 204″ to solution 504 comprising fluoroalkyl substance 506. In certain embodiments, for example, once solution 504 comprising fluoroalkyl substance 506 contacts a surface of sensing material 102′″ comprising coupled-multichromophore 104, fluoroalkyl substance 506 diffuses into coupled-multichromophore 104.

According to certain embodiments, the method comprises detecting a change in electromagnetic radiation (e.g., light) emission of at least one chromophore of the coupled-multichromophore.

In some embodiments, the detecting comprises detecting the change in electromagnetic radiation (e.g., light) emission using a detector and/or reader. Any of a variety of detectors and/or readers may be used to detect the change in electromagnetic radiation (e.g., light) emission of the at least one chromophore of the coupled-multichromophore. In certain embodiments, for example, the detector and/or reader is a fluorescence spectrometer, a photodiode, a photovoltaic device, an individual's eyes (e.g., detecting a visible colorimetric change), and/or a smartphone. Other detectors and/or readers are also possible.

According to some embodiments, as described in greater detail elsewhere herein, the at least one chromophore displays the change in electromagnetic radiation (e.g., light) emission in response to a presence of the fluoroalkyl substance.

In certain embodiments, the at least one chromophore displays the change in electromagnetic radiation (e.g., light) emission in response to protonation of the coupled-multichromophore by the fluoroalkyl substance. According to some embodiments, for example, the coupled-multichromophore comprises a moiety capable of being protonated by the fluoroalkyl substance (e.g., a N-containing moiety, such as a pyridine-containing moiety) and the fluoroalkyl substance is a Brønsted acid. In some embodiments, the detecting comprises detecting a wavelength shift in electromagnetic radiation emission of the at least one chromophore to a lower energy emission.

According to some embodiments, the detecting comprises detecting a ratiometric change in electromagnetic radiation (e.g., light) emission of at least two chromophores of the coupled-multichromophore, wherein the at least two chromophores display the ratiometric change in electromagnetic radiation emission in response to the presence of the fluoroalkyl substance. In certain embodiments, for example, the coupled-multichromophore comprises a dye (e.g., a small molecule and/or polymer, such as squaraine, oxazine, perylene bisimide, conjugates thereof, and/or combinations thereof). In some embodiments, the detecting comprises detecting a first chromophore of the at least two chromophores that displays an increase in electromagnetic radiation emission in response to the presence of the fluoroalkyl substance, and detecting a second chromophore of the at least two chromophores that displays a decrease in electromagnetic radiation emission in response to the presence of the fluoroalkyl substance.

In certain embodiments, the detecting comprises determining a concentration of the fluoroalkyl substance due to the change in electromagnetic radiation (e.g., light) emission of the at least one chromophore of the coupled-multichromophore. In certain embodiments, the change in electromagnetic radiation emission of the at least one chromophore as a function of time corresponds to the concentration of the fluoroalkyl substance detected. In some embodiments, for example, the excited state lifetime of the change in electromagnetic radiation emission of the at least one chromophore is proportional to the concentration of the fluoroalkyl substance detected. In certain embodiments, ratiometric change in electromagnetic radiation emission of the at least two chromophores corresponds to the concentration of the fluoroalkyl substance detected.

According to some embodiments, the detecting comprises comparing the change in electromagnetic radiation (e.g., light) emission of the at least one chromophore of the coupled-multichromophore to a reference signal. In certain embodiments, the reference signal has no response to a fluoroalkyl substance.

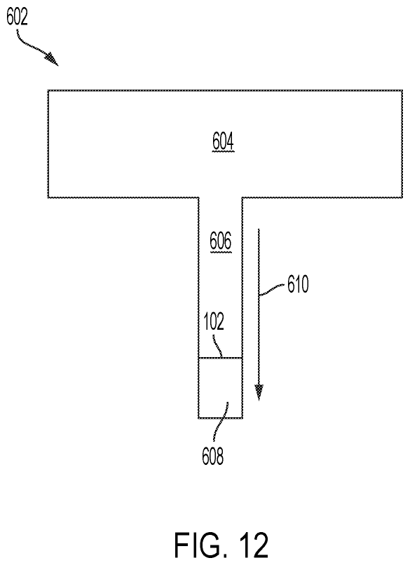

According to certain embodiments, a system is described. FIG. 12 shows, according to certain embodiments, a schematic diagram of system 602 comprising source 604 of a solution and sensing material 102 comprising a coupled-multichromophore, which is described in greater detail elsewhere herein.

Source 604 may be any of a variety of suitable sources. In certain embodiments, for example, source 604 is a well, a body of water (e.g., an ocean, a sea, a pond, a lake, a river, and the like), a residential unit, a commercial unit, an industrial plant, a semiconductor manufacturing site, a water treatment plant, a water distribution site, a food manufacturing plant, and the like. Other sources are also possible.

The solution may be any of a variety of suitable solutions, as described in greater detail elsewhere herein. In certain embodiments, for example, the solution comprises an aqueous solution (e.g., deionized water, tap water, well water, wastewater reservoir water, ocean water, sea water, pond water, lake water, river water, water from an industrial site, water from a semiconductor manufacturing site, water distributed to towns and/or cities, aqueous food solutions and the like). In other embodiments, the solution comprises a non-aqueous solution (e.g., the solution comprises an organic solvent).

In some embodiments, the solution comprises a fluoroalkyl substance (e.g., a PFAS, as described in greater detail elsewhere herein). In certain embodiments, the solution comprises one or more non-target analytes, such as one or more metal ions, polymers, biopolymers, humic acids, and/or organic oils or surfactants. Other non-target analytes are also possible.

In certain embodiments, as shown in FIG. 12, source 604 comprises fluidic outlet 606. In some embodiments, fluidic outlet 606 is configured to flow the solution from source 604 along direction 610 to outlet 608 of fluidic outlet 606.

In certain embodiments, system 602 comprises a means for concentrating the fluoroalkyl substance. In some embodiments, for example, system 602 comprises one or more means for heating the solution to concentrate the fluoroalkyl substance in the solution.

According to some embodiments, as shown in FIG. 12, sensing material 102 comprising the coupled-multichromophore may be positioned along fluidic outlet 606 such that sensing material 102 comprising the coupled-multichromophore is exposed to the solution as the solution flows from source 604 along direction 610 to outlet 608 of fluidic outlet 606. In some embodiments, for example, sensing material 102 may be configured as a film or a plurality of particles. In certain embodiments, sensing material 102 is disposed on a substrate such that the sensing material 102 disposed on the substrate is configured as a filter or membrane. In some embodiments, sensing material 102 is disposed on a plurality of particles. In certain embodiments, sensing material 102 is coated on an inner surface of fluidic outlet 606.

In certain embodiments, sensing material 102 comprising the coupled-multichromophore is configured to detect a presence of a fluoroalkyl substance in the solution, as described in greater detail elsewhere herein, as the solution flows from source 604 along direction 610 to outlet 608 of fluidic outlet 606. In accordance with certain embodiments, system 602 comprises one or more detectors and/or readers configured to detect a change in electromagnetic radiation (e.g., light) emission of at least one chromophore of the coupled-multichromophore.

In some embodiments, system 602 is configured to continuously monitor the solution for a presence of the fluoroalkyl substance. In certain embodiments, for example, system 602 may comprise one or more pumps and/or fans configured to continuously flow the solution from source 604 along direction 610 to outlet 608 of fluidic outlet 606 such that sensing material 102 comprising the coupled-multichromophore is continuously exposed to the solution. In certain embodiments, system 602 comprises one or more detectors and/or readers configured to continuously detect a change in electromagnetic radiation (e.g., light) emission of at least one chromophore of the coupled-multichromophore.

U.S. Provisional Patent Application No. 63/385,728, filed Dec. 1, 2022, and entitled “Detection of Fluorocarbon Compounds,” is incorporated herein by reference in its entirety for all purposes. U.S. Provisional Patent Application No. 63/503,979, filed May 24, 2023, and entitled “Detection of PFAS,” is also incorporated herein by reference in its entirety for all purposes.

The following example is intended to illustrate certain embodiments of the present invention, but does not exemplify the full scope of the invention.

### Example 1

Herein it is shown how the self-amplifying properties of fluorescent conjugated polymers enable the detection of aqueous PFAS at ng/L. Signal amplification in coupled amplifying fluorescent polymer (AFP) systems is the result of highly efficient excited state (exciton) transport along the polymer backbones and between neighboring polymers. The rapidly diffusing excitons in some cases can sample thousands of polymer repeating units increasing the probability of encountering an analyte of interest (PFAS in the present case). If the presence of the analyte causes a lower energy trapping state of a quenching site, the exciton is captured. The utility of AFPs has been demonstrated in a diverse array of chemical and biological sensing applications, and in some cases these methods have proved to be robust enough to be implemented in commercial sensing devices.

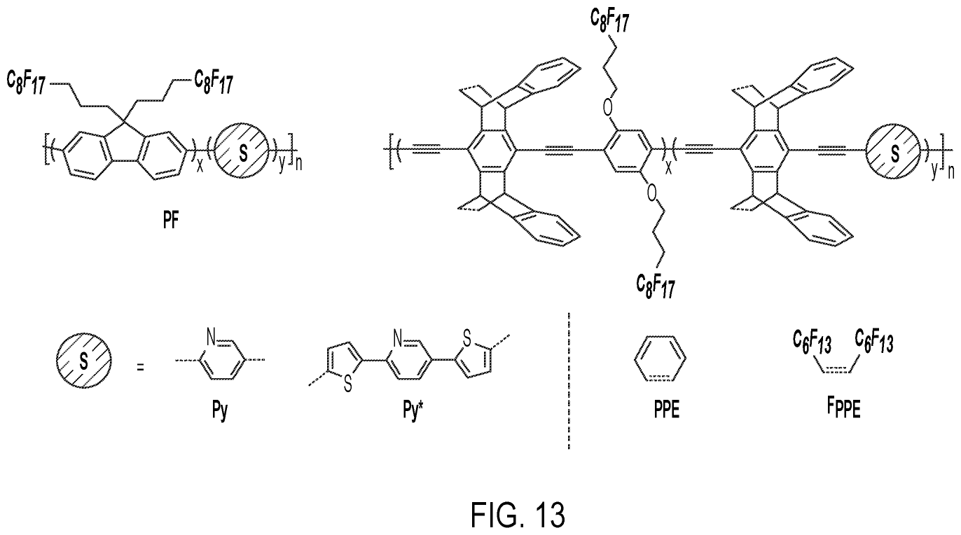

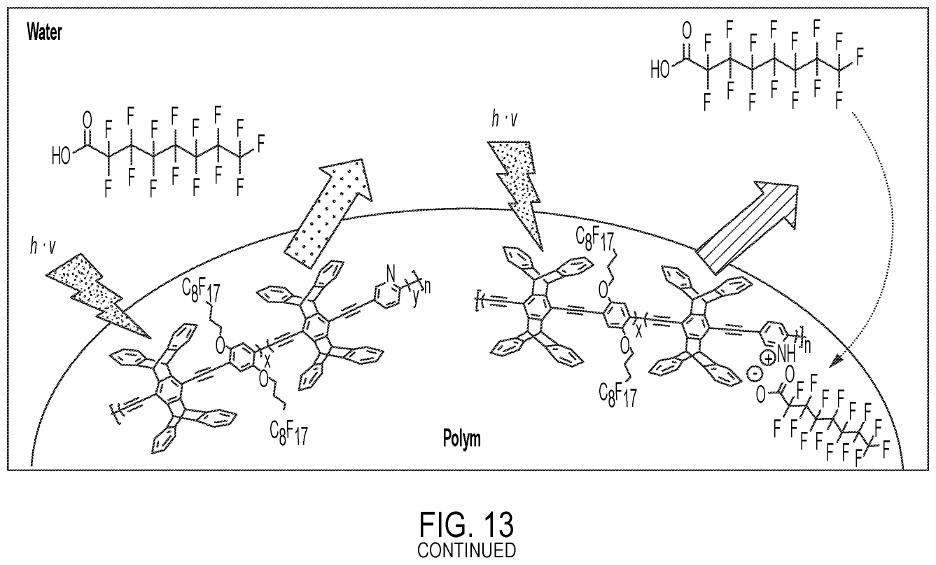

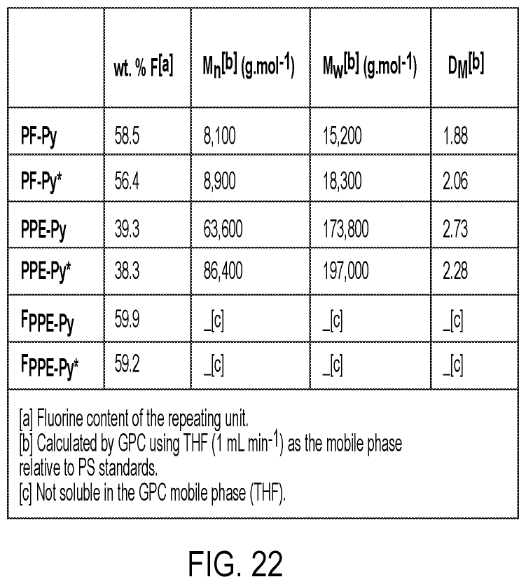

A new sensing platform based on fluorescent polymers that specifically bind and respond to PFAS in aqueous environments is reported. The method relies on highly fluorinated polymers of the poly(p-phenylene ethynylene) (PPE) and polyfluorene (PF) motifs bearing pyridine-based selectors that react with acidic PFAS (i.e., PFOA and PFOS) via a proton-transfer reaction (see FIG. 13). The fluorinated domains within the polymer backbone partition PFAS into polymers, while the protonated pyridine units produce new emissive signals that are amplified by excitonic energy transport. Specifically, two acidic PFAS selectors were designed (Py and Py* shown in FIG. 13), where the π-electron delocalizing character of the thiophene bridges in Py* triggers larger changes in fluorescence after protonation, in comparison to the simple pyridine selector (Py). As for the polymer backbones, three different polymers were selected (PF, PPE, and ^{F}PPE shown in FIG. 13), which impede aggregation, allowing for spectroscopic stability and high emission efficiency in thin films and particle forms. In the case of PPE and ^{F}PPE polymers, the rigid pentiptycene repeating units introduce molecular-level porosity that facilitates PFAS diffusion into solid polymers. Moreover, both PF and ^{F}PPE polymers possess a particularly high fluorine content (see FIG. 22) that increases PFAS affinity for the polymers.

### Results and Discussion

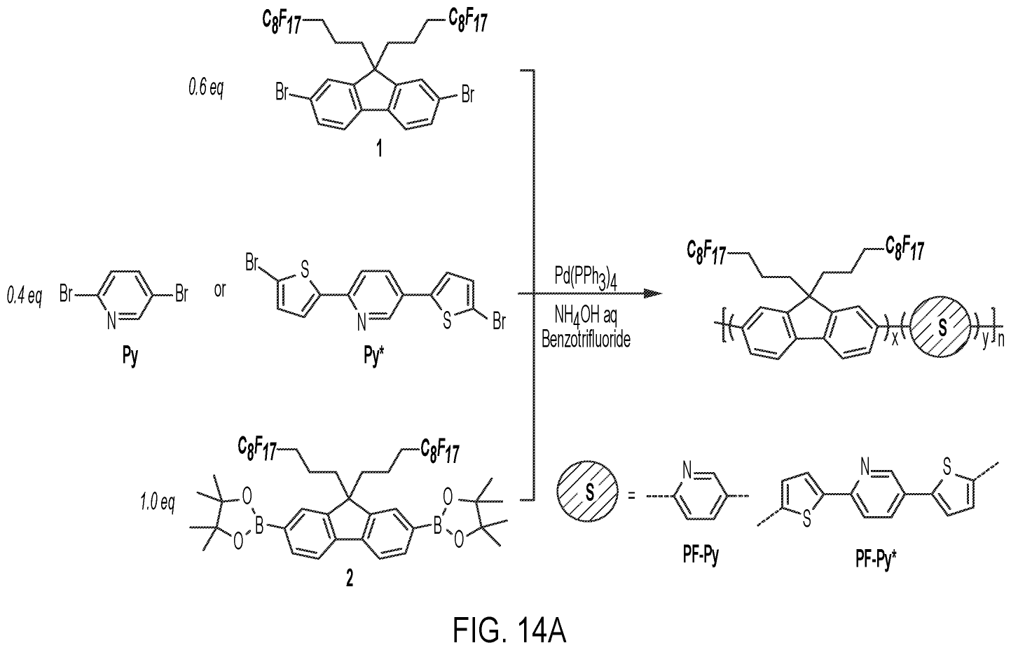

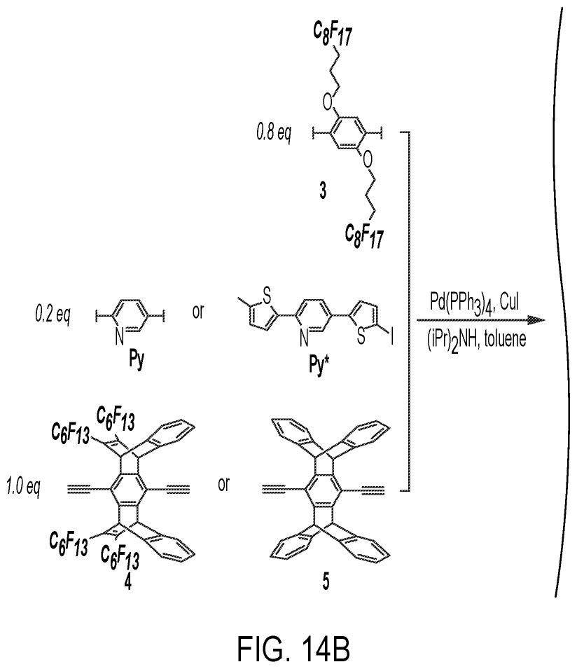

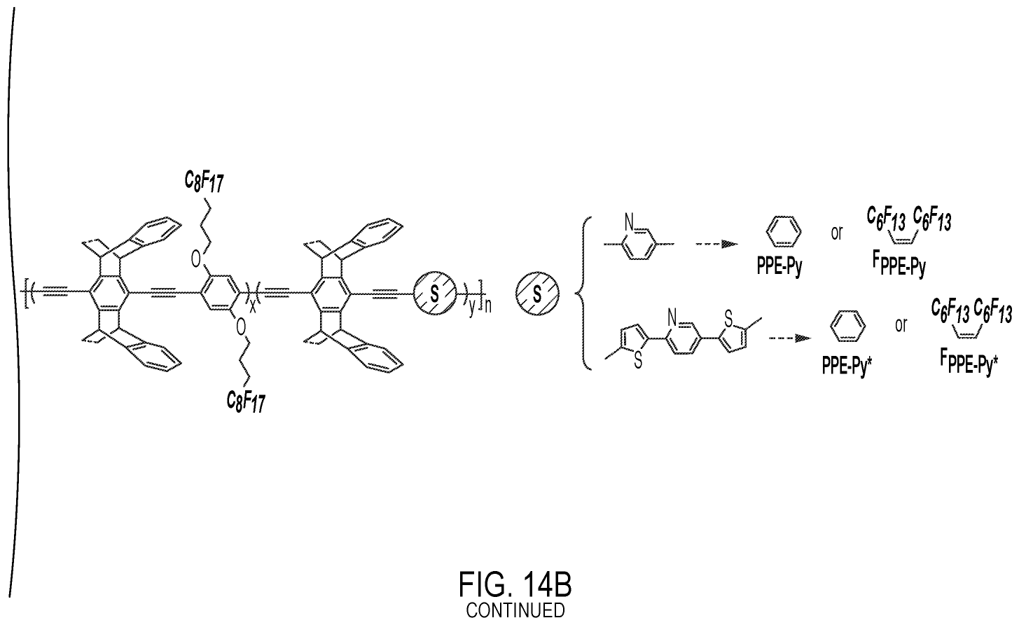

AFPs were synthesized by palladium-catalyzed cross-coupling polycondensation. PF-Py and PF-Py* were prepared via Suzuki polymerization between dibromide 1, diboronate 2, and the pyridine-containing dibromide (Py or Py*) (see FIG. 14A). Although all the monomers were soluble in toluene, the Suzuki polymerization was performed in benzotrifluoride to solubilize the generated polymer that has fluorous characteristics. In contrast, PPE polymers were synthesized by Sonogashira polymerization between diethylnyl [2.2.2] bridged bicyclic monomers 4 or 5, diiodide 3, and pyridine-containing diiodide (Py or Py*) in benzotrifluoride/diisopropylamine (3:2) (see FIG. 14B). All the polymers were purified by precipitation in methanol, followed by repeatedly washings with hot methanol, acetone and acetonitrile. The relative molecular weights and polydispersity indices were estimated by gel permeation chromatography (GPC) in THE solution using polystyrene standards (see FIG. 22). However, ^{F}PPE polymers were only soluble in fluorinated solvents, such as benzotrifluoride and hydrofluoroethers (e.g., HFE-7500 or HFE-7200), thereby preventing molecular weight determination GPC. To evaluate the molecular weight, dynamic light scattering (DLS) in benzotrifluoride solution was used to calculate their radius of gyration. DLS analysis suggested an average radius of 20.8 nm for PPE-Py and 21.6 nm for ^{F}PPE-Py*, which are slightly larger than the persistence length of high molecular weight PPEs.

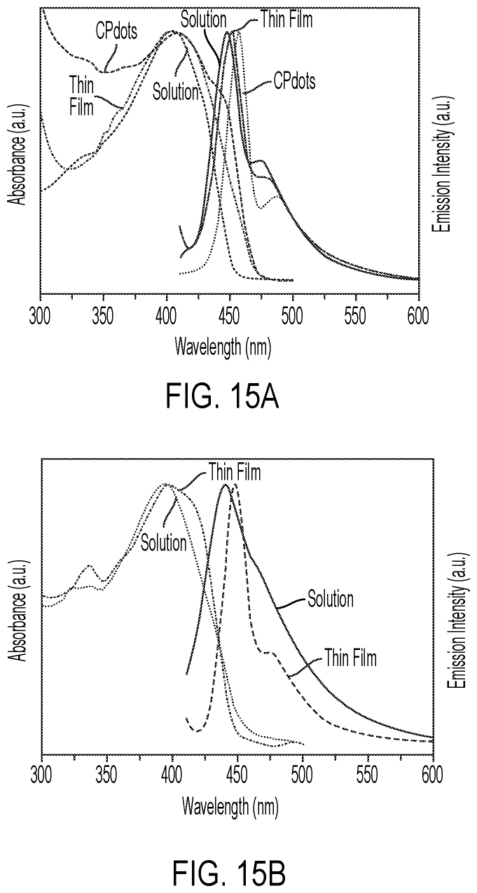

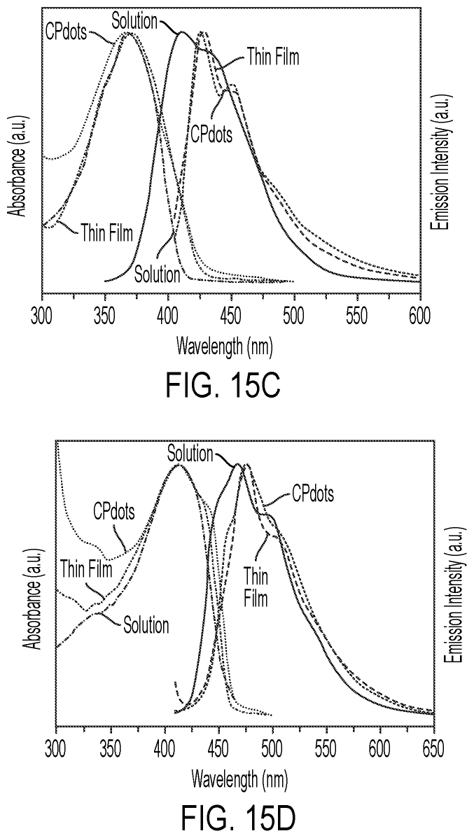

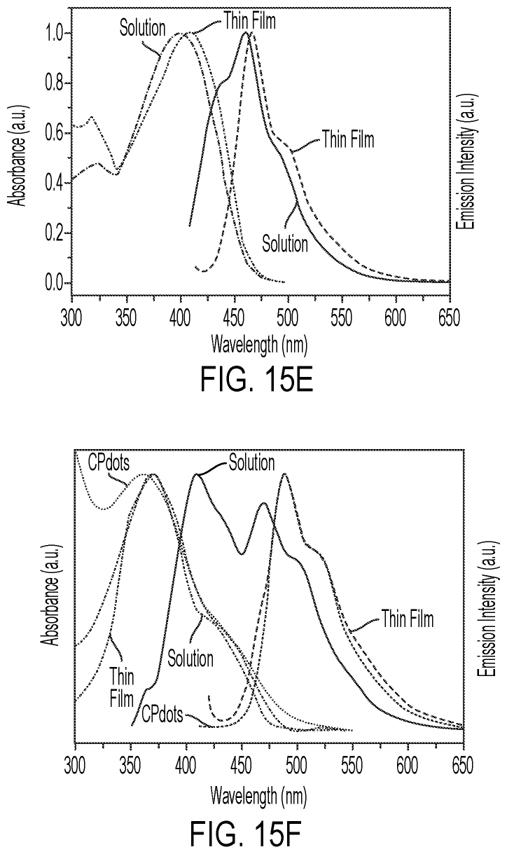

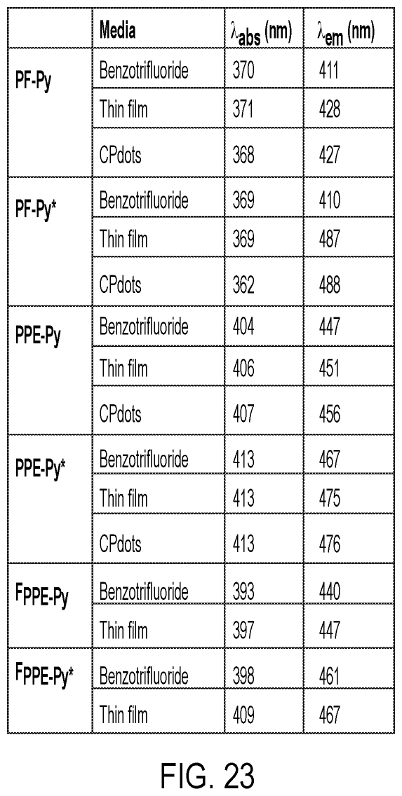

The AFP UV-Vis absorption and fluorescence spectra were collected in dilute solutions of benzotrifluoride and in spin-casted films (see FIGS. 15A-15F). Relevant photophysical data were collected (see FIG. 23). The fact that the absorption and fluorescence profiles vary little between solution and thin films suggests weak interpolymer interactions and confirms that the absence of aggregation prevents self-quenching to maintain high emission quantum yields in thin films/particles and makes for high reproducibility of spin-casted films. In the case of the PFs, some minor aggregation was observed, but overall the perfluoroalkane chains that extend perpendicular to the fluorene repeating units prevent close stacking of the conjugated backbones.

The fluorescence response of polymer thin films (30 to 50 Å thick) to aqueous solutions of PFOA was studied by introducing the films into sealed vials (20 mL capacity) containing 10 mL of different concentrations of PFOA in milliQ water. The mechanism of fluorescence change is the protonation of a nitrogen atom of a Lewis base (pyridine) and proton-transfer reactions are considered to occur nearly instantaneously, as a result the most relevant and time-limiting factor that contributes to the fluorescence response is the time needed for PFOA molecules to diffuse from water to the fluorophilic polymer films.

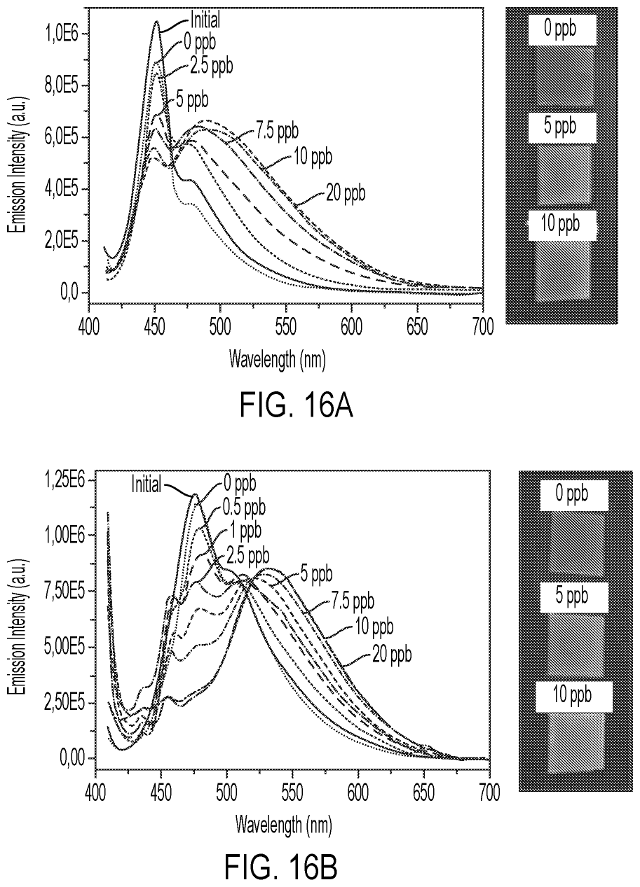

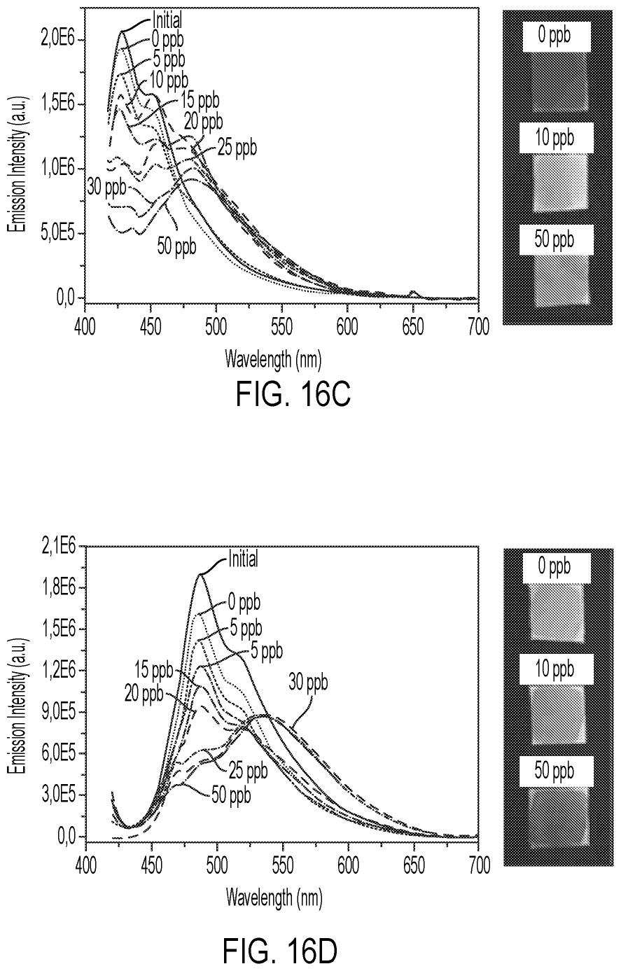

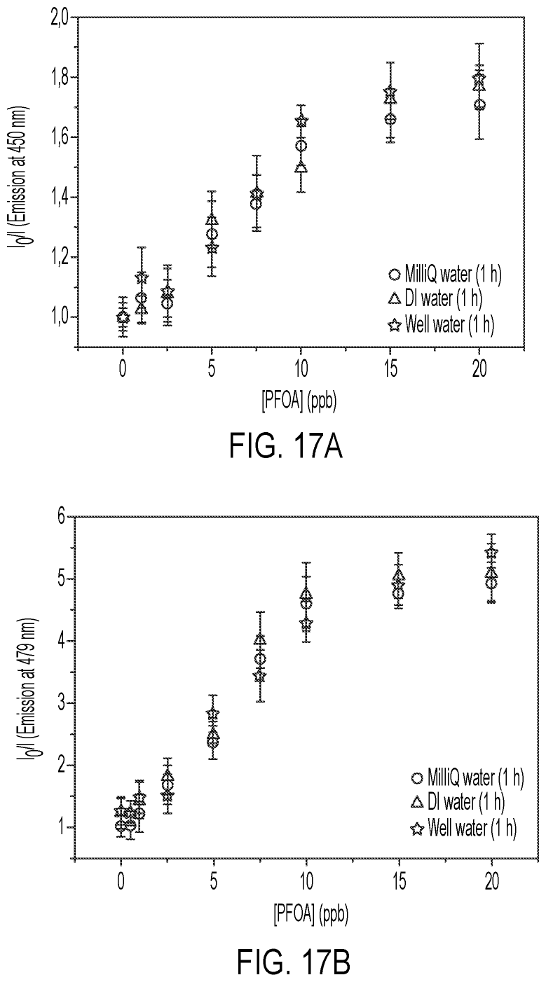

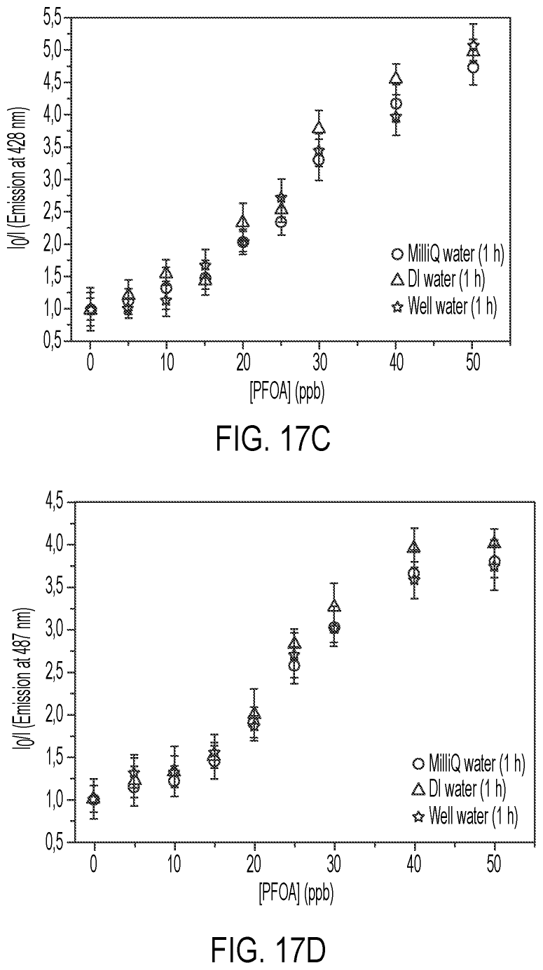

To ensure adequate PFAS diffusion into the films, an exposure time of 1 hour was used to perform all the sensing experiments. Exposure of PPE-Py films to PFOA results in a broadening and red-shifting of the emission peak (from 451 to 490 nm), and visually the films change from having a blue to a bluish-green fluorescence (see FIG. 16A). This red shift is the result of the PFOA-induced protonation of pyridine units, which produces enhanced electro-accepting character and lowers the energy of charge transfer states. As a result of the amplifying nature of exciton migration, only a small percentage of pyridine acceptors need to be protonated to produce a large response. Thus, the short-wavelength shoulder of the initial fluorescence band probably suggests some residual emission from non-protonated PPE-Py. The PPE-Py* polymer showed a similar PFOA response, but the thiophenes produce a stronger change in emission with PFOA-induced protonation of pyridine units (see FIG. 16B). In particular, PPE-Py* films exhibited a blue to green visual fluorescence color change, together with a larger red-shifting of the initial band from 475 to 535 nm. The larger shift was expected as the thiophene-pyridine constructions were expected to have strong charge transfer character. FIGS. 17A-17D show the calibration curves for both polymers, which are linear between 1 and 10 ppb. The limits of detection (LOD) were calculated to be 2 ppb (PPE-Py) and 1 ppb (PPE-Py*). Moreover, it was confirmed that film recycling was possible and most of the initial fluorescence of PPE-Py and PPE-Py* films can be recovered by rinsing with an aqueous NaOH (1 M) solution for 10 minutes and air drying.

^{F}PPE polymers are highly fluorinated AFPs with fluorine content of 40 to 60 wt. % (see FIG. 22). It was initially hypothesized that ^{F}PPE will exhibit higher sensibility as a result of the higher partitioning of PFAS into a more fluorous polymer film. More than two orders of magnitude lower sensitivity was observed with LOD˜100 ppb relative to other PPE analogues. This considerably lower PFOA response is consistent with ^{F}PPE's highly hydrophobic nature, and the poor wettability of the polymer film likely reduces PFOA diffusion into the film.

Exposure to PFOA causes the emission bands of PF-Py and PF-Py* to be broader, less-intense, and red-shifted. Bands shifted from 428 to 482 nm for PF-Py and from 487 to 535 nm for PF-Py* (see FIGS. 15C-15D). As revealed by the photographs of the thin films in FIGS. 16A-16D, polymer films also showed visual detectable fluorescence color changes in response to PFOA. The evolution of the fluorescence intensity at the initial peak maximum for PF-Py and PF-Py* upon exposure to different PFOA concentrations (calibration curves) revealed linear regions from 10 to 40 ppb with calculated LOD of 8 ppb for PF-Py and 6 ppb for PF-Py* (see FIGS. 17A-17D).

It is worth noting that PF-Py/PF-Py* and PPE-Py/PPE-Py* AFPs have LOD of the same order of magnitude. This fact suggests that the diffusion of the PFOA into the polymers may be a limiting factor. Interestingly, PPE-Py/PPE-Py* and PF-Py/PF-Py* films did not exhibit fluorescence response when exposed to aqueous solutions of simple octanoic acid, thereby demonstrating the that fluorinated segments within the AFPs bind to PFAS. The AFP-based sensor scheme relies on a relatively non-specific proton-transfer reaction and might be inherently susceptible to interferences from acidic and/or ionic species commonly found in groundwater. As a result, it was decided to evaluate the potential interfering issues that may arise with complex aqueous matrices, such as groundwater. In particular, well water collected from a well in Central Vermont was selected as a realistic matrix to demonstrate the robustness of the AFP-based sensor. FIGS. 17A-17D show the PFOA calibrations curves when the polymer films were immersed in milliQ water, DI water, and well water. Only minor deviations were observed in the fluorescence responses to PFOA, and also the same detection limits within the margin of error.

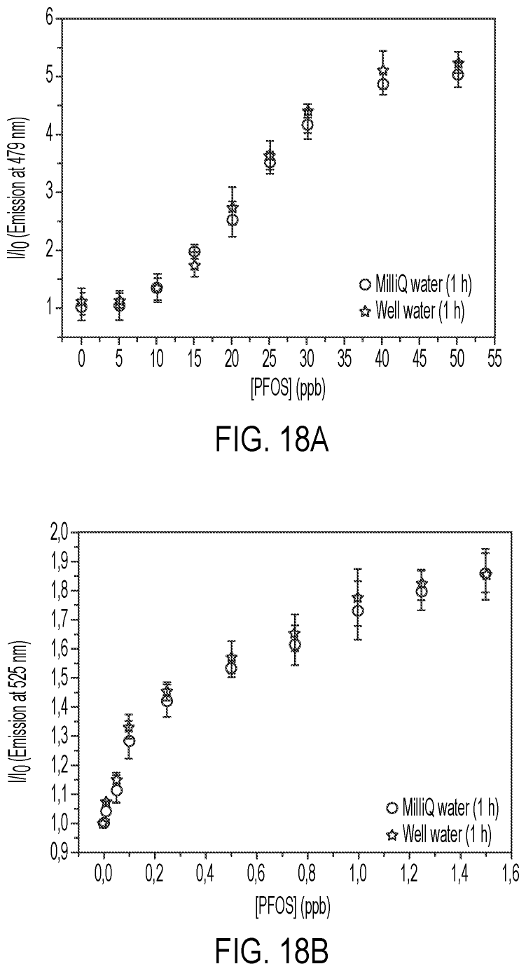

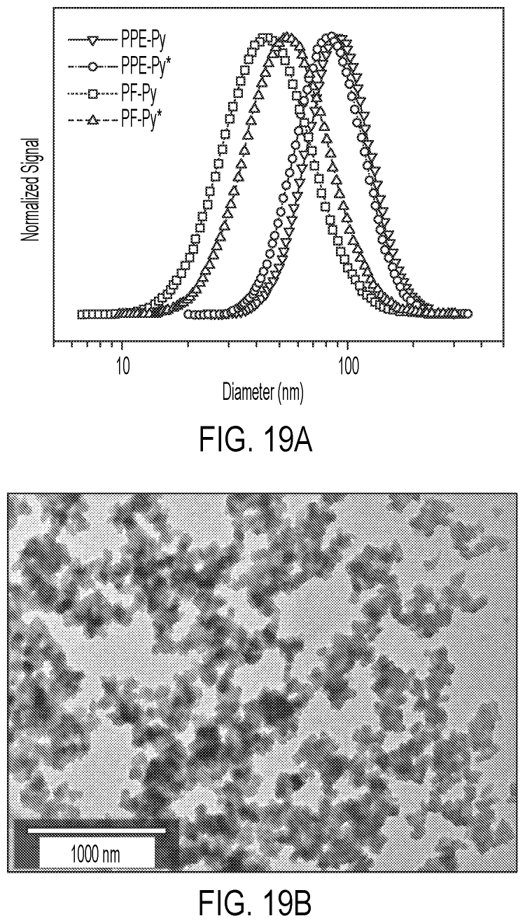

It was wondered if the method detection of PFOA had general utility for sensing of other acidic PFAS, such as PFOS. PPE-Py* is the AFP with the lowest LOD and its fluorescence response to PFOS was studied. As shown in FIGS. 18A-18B, a clear fluorescence change was observed after exposing PPE-Py* polymer films to PFOS in milliQ and well water. The calibration curve reveals a linear region from 10 to 40 and a calculated LOD of 5 ppb. The higher LOD for PFOS (i.e., 5 ppb for PFOS and 1 ppb for PFOA) may be a result of the higher affinity of the sulfate, relative to a carboxylate, to be hydrated. It is likely that PFOS behaves as a surfactant at fluorous/water interface. PFOS organized at the interfaces will not protonate the pyridines and lead to a reduced LOD.

It is likely that the AFP sensing response is related to the diffusion of PFOA from water into the polymer film, which is dependent on the polymer/water interfacial area. Spin-cast AFP films have a limited surface area and to increase the polymer/water interface conjugated polymer nanoparticle (CPdot) dispersions were prepared in water. CPdots have found applications in imaging and chemical sensing and can be prepared by a reprecipitation method. Briefly, a dilute solution of the AFP in THF (0.01 mg·mL⁻¹, 2 mL) was quickly added to water (8 mL) under sonication. THF was then evaporated under vacuum to yield optically clear aqueous dispersions of CPdots that display the same color as the starting THE solution. ^{F}PPE-Py and ^{F}PPE-Py* are not soluble in non-fluorinated solvents, complicating the formation of CPdots. As a result, CPdot studies focused on CPdots of PPE-Py, PPE-Py*, PF-Py, and PF-Py*. The resulting CPdots dispersions were stable over 1 month with no evidence of aggregation nor precipitation. The morphology of the CPdots and their size was evaluated by transmission electron microscopy (TEM) and DLS (see FIGS. 19A-19B). TEM images showed the presence of spherical nanoparticles, which appeared to aggregate into interconnected networks. This aggregation has been previously observed for CPdots, and occurs during water evaporation due to the high hydrophobicity of the fluorinated polymers. In contrast, no aggregation of CPdots was detected in DLS experiments (PDI<0.20), which gave monomodal size distributions with mean hydrodynamic diameters of 92 nm (PPE-Py), 83 nm (PPE-Py*), 54 nm (PF-Py), and 44 nm (PF-Py*).

The UV-Vis absorption spectra of the aqueous dispersions of the CPdots were broadened compared to those of the conjugated polymers in benzotrifluoride solution (see FIGS. 15A-15F). Nonetheless, the absorption spectra of the PF-Py/PF-Py* CPdots have a slight blue-shifting from the solution state that is consistent with an overall reduction of the conjugated length of the AFP chain. In contrast, the absorption spectra of PPE-Py/PPE-Py* CPdots did not show blue-shifting. CPdots also exhibited red-shifted fluorescence spectra as compared to those of in solution, which is very similar to the spectra acquired in thin film form.

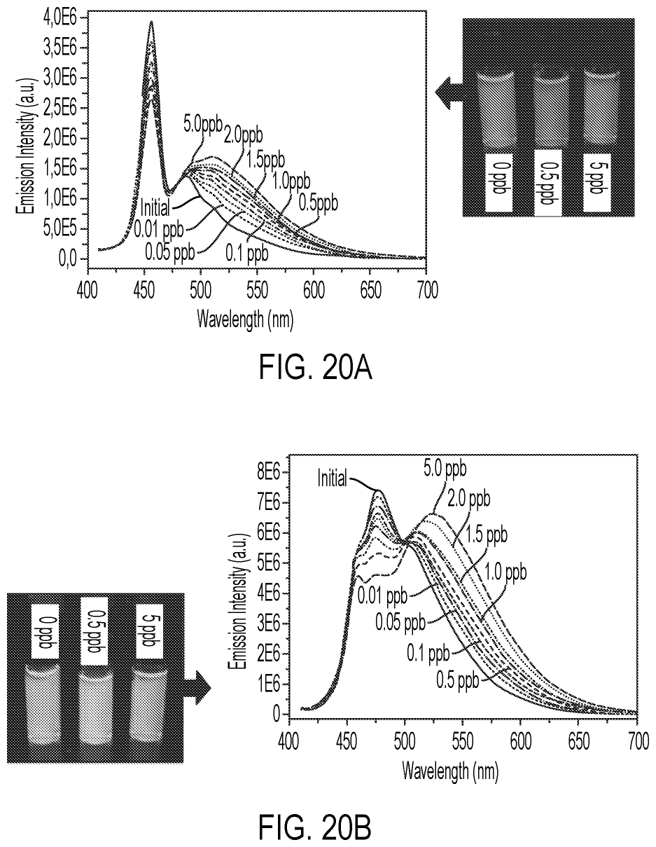

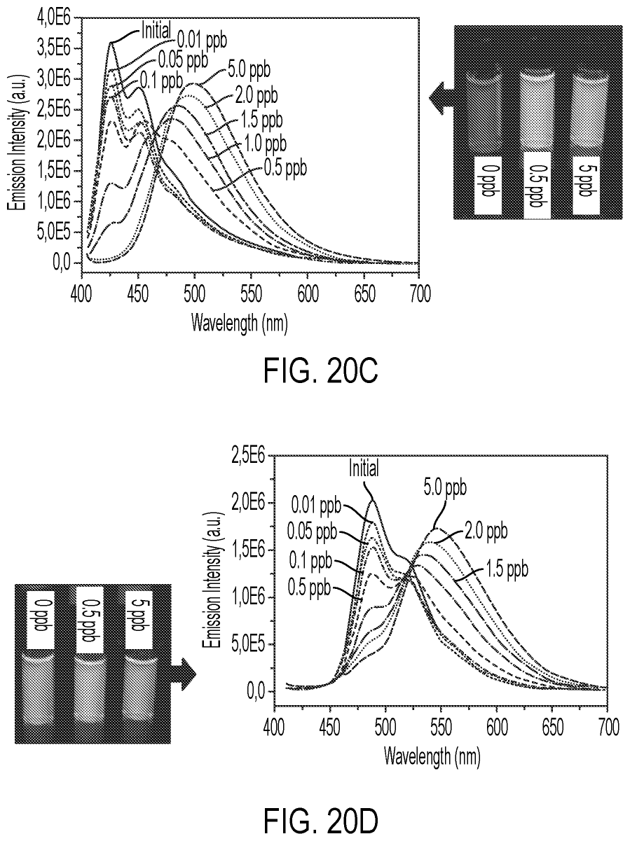

The fluorescence spectra of the CPdots were recorded after 1 hour incubation with different concentrations of PFOA in milliQ water (see FIGS. 20A-20D). In accord with the thin film sensing experiments, PFOA exposure resulted in a broadening and red-shifting of the emission peaks. Moreover, those changes in the fluorescence spectra were accompanied by a visual change in the fluorescence color of the CPdot aqueous dispersions. These results confirmed that PFOA is able to diffuse into the CPdots and protonate the pyridine-based selectors, triggering changes in the fluorescent properties. FIGS. 20A-20D show the calibration curves for the CPdots that reveal linear regions from ca. 0.05 to 1.5 ppb. A LOD of 0.2 ppb was calculated for PPE-Py, 0.08 ppb for PPE-Py*, 0.8 ppb for PF-Py, and 0.7 ppb for PF-Py*. These values are approximately one order of magnitude lower than those of found in thin film experiments, attesting to the impact of higher surface area.

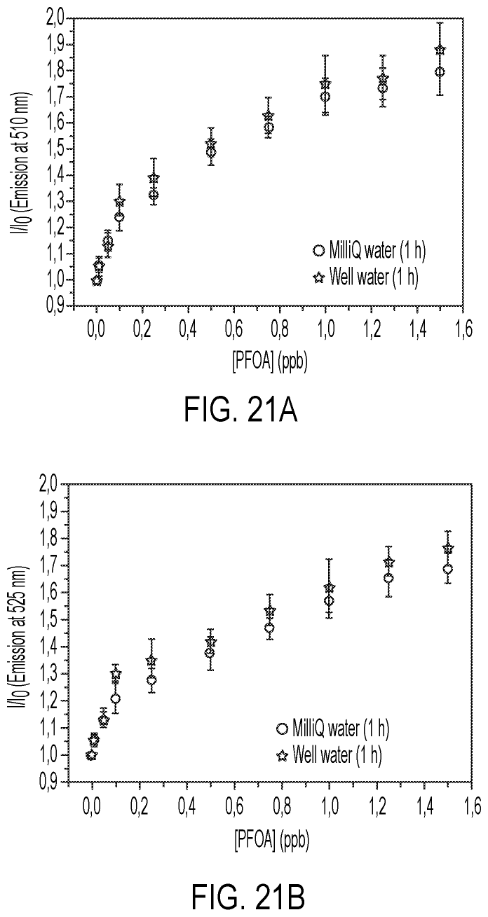

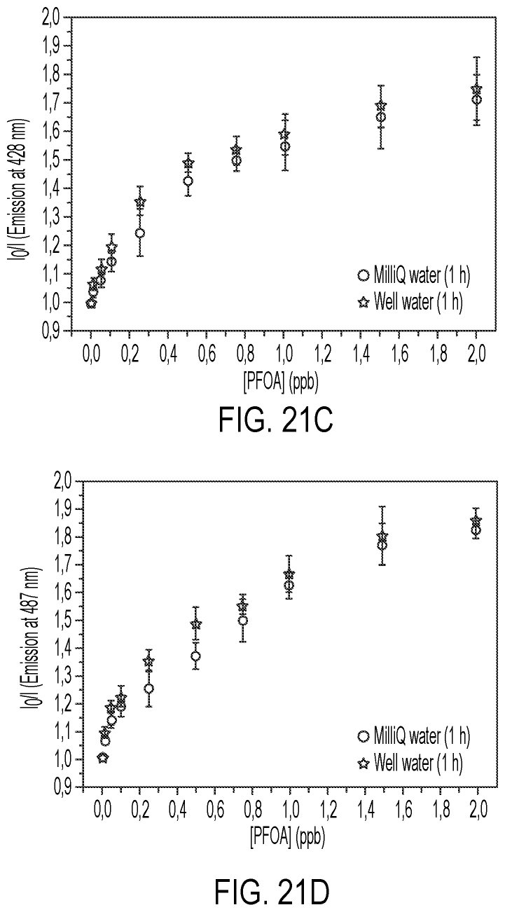

It was also determined that the CPdots performance is the same in milliQ water and well water (see FIGS. 21A-21D). CPdots-based AFP sensors are also able to detect PFOS in addition to PFOA. CPdots of PPE-Py* showed a fluorescent response upon exposure to different concentrations of PFOS (see FIGS. 18A-18B). The calibration curve for this data is linear from 0.1 to 1.5 ppb and gives a calculated LOD of 0.35 ppb. As described, the higher LOD for PFOS is likely related to its different interfacial activity in comparison to PFOA.

### Conclusion

In conclusion, amplifying fluorescent polymers (AFPs) that can selectively detect aqueous PFOA and PFOS in the nanogram range have been developed. The AFPs are highly fluorinated and have poly(p-phenylene ethynylene) and polyfluorene backbones. Pyridine-based selectors were integrated into the AFPs that react with acidic PFAS acids via a proton-transfer reaction. PFAS-induced protonation of the pyridines creates lower-energy pyridinium traps for the excitons and emission from these sites results in a red shift of the spectra. These AFPs were initially evaluated as spin-coated films and can detect PFAS at concentrations of ˜1 ppb. Higher surface area nanoparticles can detect aqueous PFAS concentrations of ˜100 ppt. It is also noteworthy that both polymer films and CPdots are not affected by the type of water, and similar responses to PFAS were found in milliQ water, DI water, and well water. The low detection limits, in addition to the relatively rapid response (˜1 hour), makes this sensor scheme potentially suitable for on-site PFAS detection.

### Example 2

Disclosed herein are materials containing perfluoroalkane groups. The perfluoroalkane segments can be bounded by other groups including oxygen, nitrogen, halides, methylenes, alkenes, carboxylates, and sulfates. Collectively these materials are often referred to as perfluoroalkane substances, or PFAS. Two noteworthy PFAS molecules are perfluoro-octanoic acid (PFOA) and perfluoro-octane sulfate (PFOS). The term analyte is often used in the context of sensing and is the substance that is being detected. PFAS is an example of an analyte.

A sensing composition disclosed herein refers to a material that changes its emissive characteristics in response to exposure to the PFAS analyte. The sensing composition has an affinity for the PFAS analyte and this affinity is induced by the incorporation of perfluoroalkane units in the sensing composition.

Disclosed herein are electronically active polymers with small molecule dye guests that have perfluoroalkyl groups capable of absorbing perfluoroalkane substances (PFAS) (analytes) from water and in doing so create an optical response that can be used to determine the absence or presence of this environmental pollutant. The polymers are capable of facile energy migration and in some cases have conjugated backbones and the polymers transfer energy to the guest molecules to give optical emission at a longer wavelength than the optical emission of the polymer. The polymers can be coated on solid supports or in particle form. The absorption of PFAS from water will change the emission profile of the composition to provide a signal that indicates the presence of the analyte.

The sensing materials rely on the ability of the dyes to form strong electronically coupled complexes to the host polymers. A dye is a molecule capable of absorbing light in the ultra-violet and visible range of the electromagnetic spectrum. A dye may be emissive and capable of creating a new emission by binding it to another chromophoric material. Alternatively, the dye can be non-emissive. The dye in this sensing composition can display changes in its ability to accept energy, change its absorption of emission characteristics, or change its interactions with other chromophores in response to the presence of the PFAS analyte. The electronic coupling mediates energy transfer from the polymer to the dye; the electronic coupling can also create new electronic states that have composite character of the dye and the polymer. In some cases, the new electronic states are described as an exciplex and these types of emissive species often are formed between two chromophoric systems that have complementary electron donating and electron accepting character. In some cases, the PFAS molecule analytes will enhance the interaction between a donating chromophore and an accepting chromophore by a hydrogen bonding or proton transfer interaction. In some embodiments, the accepting chromophore will have a Brønsted basic site that will interact with an acidic PFAS molecule, like perfluoro-octanoic acid or perfluorosulfonic acid. This responsive acceptor molecule can be the small molecule dye or the polymer. If the donor-acceptor interactions are enhanced by the PFAS a new emission can be observed, which in some cases will be characterized as an exciplex.

In other cases, the donor molecule can be modulated by interaction with an acidic PFAS molecule. A polymer-dye composition may display an emission that is a composite of both components. In some cases, this will be considered an exciplex emission. If the donor dye can interact with the PFAS in a hydrogen bond or proton transfer, its donating ability can be reduced such that it the nature of the emission from the material changes.

The polymer-dye will display a different emission characteristic in response to PFAS that can be used to detect its presence and concentration. The characteristic can include changes in emission intensity as a function of wavelength. In some cases, the characteristic used to detect PFAS is the ratio of intensity of different emissions from the polymer-dye composition. In some cases, the characteristic used to detect PFAS is a change in the excited state lifetime. Changes in the excited state lifetimes that are longer can be used with delayed acquisition methods to detect emissive signals without prompt fluorescence backgrounds.

Optical measurements can be made from coatings of the polymer-dye sensing compositions on surfaces. In some cases, the surfaces can comprise a fiber optic, in some cases the surface can comprise glass, in some cases the surfaces can be an optical wave guide, in some cases the surfaces can be plastic, in some cases the surfaces can be paper, and in some cases the surface can be a filter. The polymer-dye sensing composition can be coated on particles. In some cases, the particles are formed exclusively from the polymer-dye sensing composition. In some cases, the sensing polymer-dye composition is coated on another particle. Particles have a high surface area and when mixed in water can allow for effective concentration of the PFAS in the particles from the water. The particles can be denser or lighter than water, which can allow for them to float to the surface or sink to the bottom of a vessel filled with water after mixing is stopped. Isolating particles at interfaces can facilitate measurement of their emissive characteristic that is associated with PFAS detection. The polymer-dye sensing composition can also be coated on magnetic particles that can be localized at interfaces by application of a magnetic field.

A PFAS detection mechanism was selected that is based on an electronic energy transfer (ET) interruption, in which a fluorescent conjugated polymer acts as a light-harvesting unit (donor) to amplify the emission from a dye (acceptor). There are two possible mechanisms for ET in thin film, a Förster resonance energy transfer (FRET), and an electron-exchange pathway formulated by Dexter. The former is a long-range dipole-dipole interaction that mainly depends on the spectral overlap between the two components, whereas the latter requires efficient π-π interactions between the conjugated polymer and the acceptor to enable orbital overlapping, and is extremely sensitive to intermolecular distance changes of only few angstroms. In this context, a selective sensor scheme to detect cyclic ketones via exchange-based ET was previously described, wherein small binding interactions between the analyte and a dye cause small movements (0.5-2 Å) of the dye that diminish-orbital overlapping, leading to a quench in the emission of the dye and to an increase in the polymer emission.

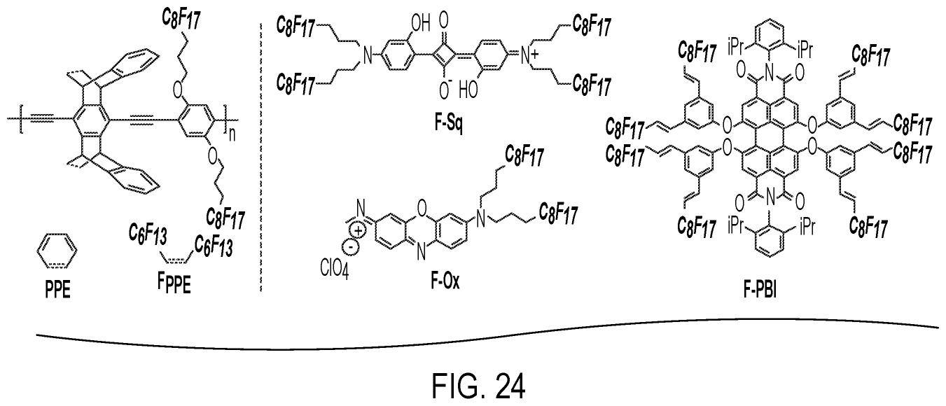

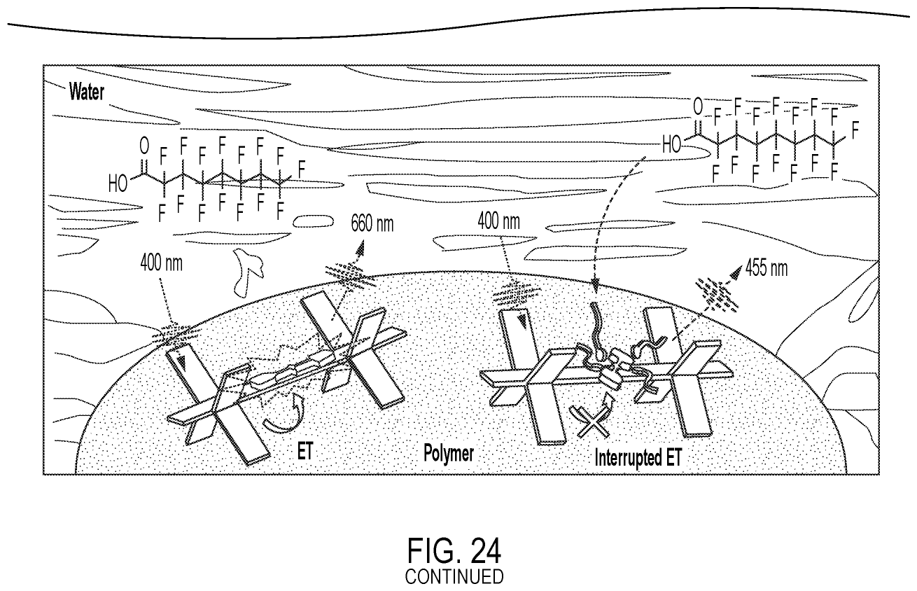

Disclosed herein is a ratiometric and selective sensing approach to detect PFAS in aqueous environments through interrupted exchange-based ET. The method relies on the ability of a highly fluorinated a poly(p-phenylene ethynylene) to amplify the emission of an embedded fluorinated fluorophore (see FIG. 24). The fluorinated domains within the polymer backbone partition PFAS into polymers, and the rigid pentiptycene repeating units introduce molecular-level porosity that also facilitates PFAS diffusion into the solid polymers. Exposure to aqueous solutions of PFAS produces a small displacement of the dye from the polymer backbone, thereby diminishing the efficiency of the ET and producing a pseudo-ratiometric fluorescent response (“polymer-ON/dye-OFF”). Specifically, two polymers were synthesized, PPE and ^{F}PPE, wherein ^{F}PPE polymer possesses a particularly high fluorine content (61 wt. %) in comparison to PPE (43 wt. %), which potentially increases PFAS affinity for the polymer. As for the acceptors, three fluorinated dyes were selected, a squaraine (F-Sq), an oxazine (F-Ox), and a perylene bisimide (F-PBI) derivatives, which are known to have the negligible spectral overlap with the light-harvesting polymers to undergo ET through an electron exchange mechanism.

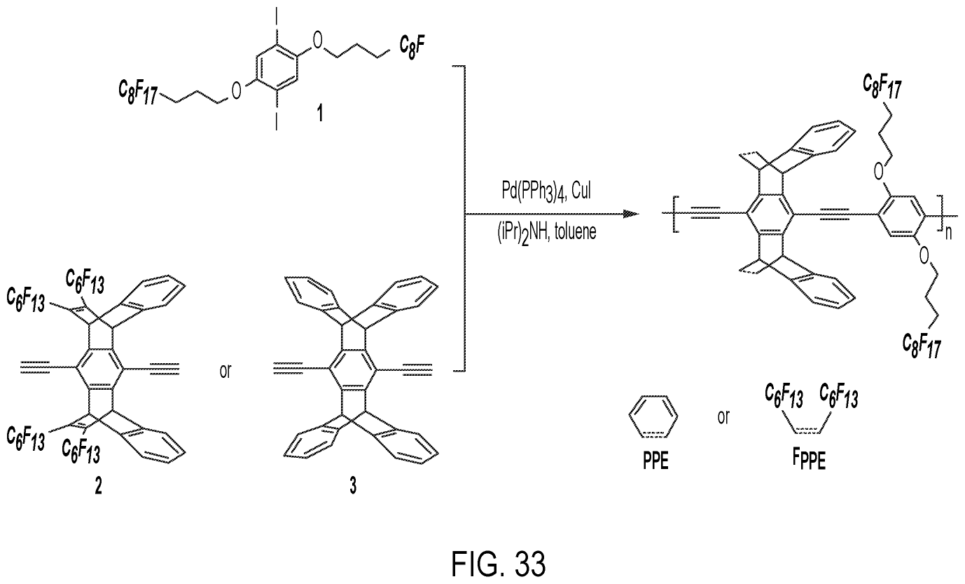

The fluorous squaraine (F-Sq), oxazine (F-Ox), and perylene bisimide (F-FBI) were synthesized following previously reported procedures. The conjugated polymers, PPE and ^{F}PPE, were synthesized by palladium-catalyzed Sonogashira polymerization between diethylnyl [2.2.2] bridged bicyclic monomers, and a diiodide in benzotrifluoride/diisopropylamine (3:2) (see FIG. 33). Both polymers were purified by precipitation in methanol, followed by repeatedly washing with hot methanol and acetone. The average molar masses and polydispersity indices were estimated by gel permeation chromatography (GPC) in THE solution using polystyrene standards. Nonetheless, the exclusive solubility of ^{F}PPE in fluorinated solvents prevented the determination of its molar mass by GPC. Therefore, dynamic light scattering (DLS) was used to estimate the length distribution of ^{F}PPE. DLS measurements in benzotrifluoride solution showed an average radius of gyration of 18.7 nm, which approximately corresponds to the typical persistence length of high-molecular weight poly(p-phenylene ethynylene) s.

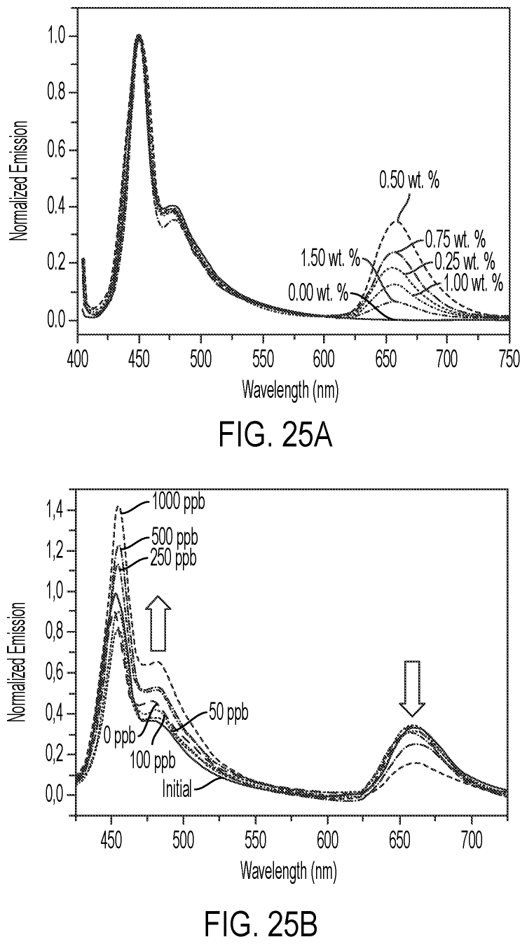

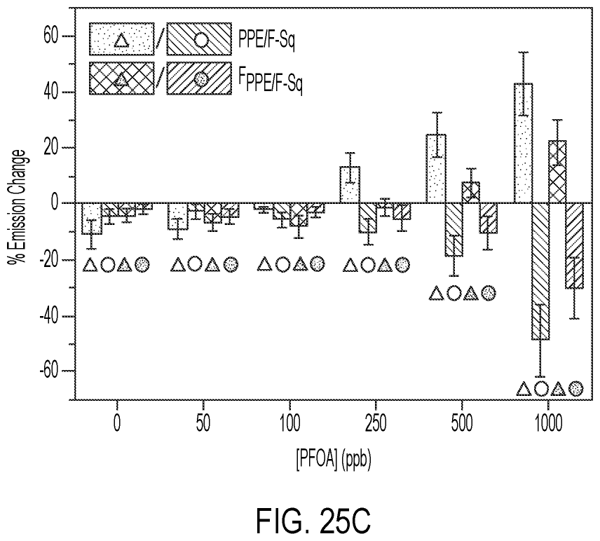

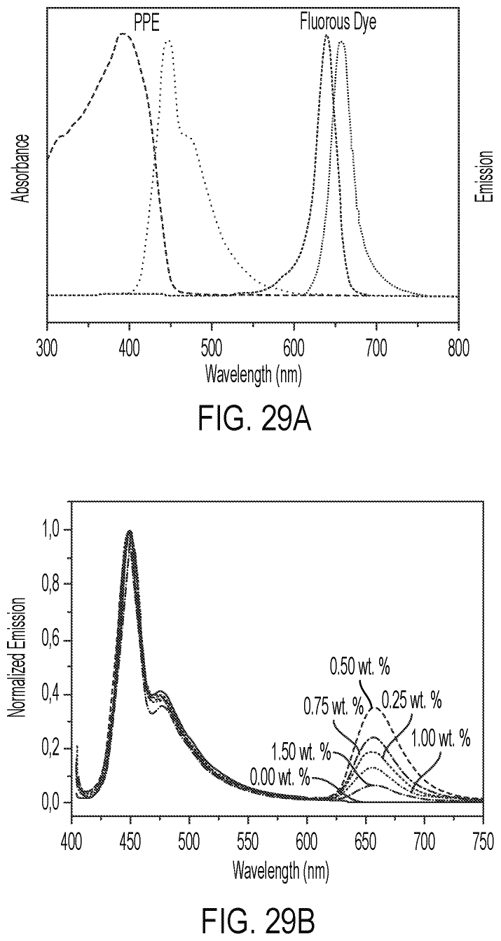

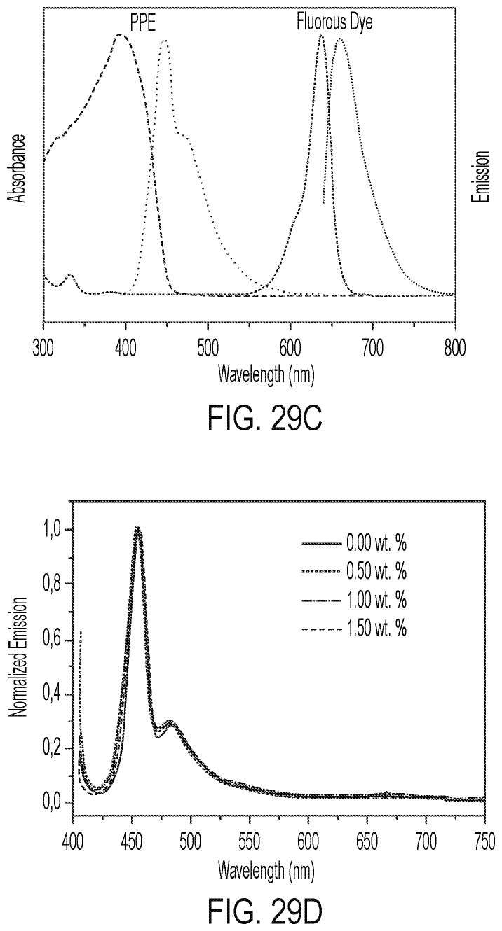

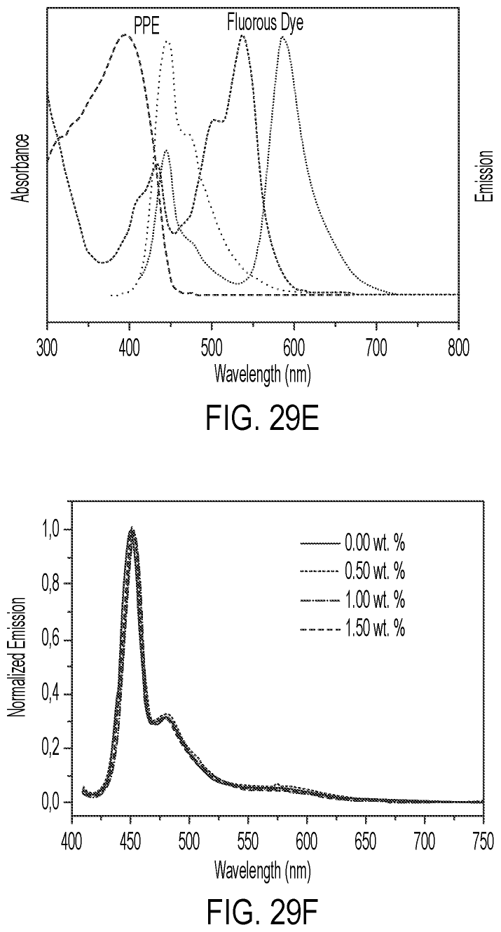

To optimize the sensor formulations, several weight ratios of the dye (F-Sq, F-Ox, or F-PBI) were dispersed in the polymer (PPE or ^{F}PPE). Briefly, the required amounts of the polymer and the dye were weighed and dissolved in benzotrifluoride, followed by spin-casting onto clean glass substrates. The fluorescence spectra of these thin films showed that F-Ox and F-PBI displayed a negligible ET when dispersed in a thin film of PPE or ^{F}PPE (see FIGS. 29A-29F), whereas F-Sq undergoes facile energy transfer with both conjugated polymers (see FIG. 25A). Given the negligible spectra overlap between PPE/^{F}PPE and F-Sq (see FIGS. 29A-29B), the observed ET process is more consistent with an exchange mechanism, in which there is an electron exchange from the excited polymer to the excited state of the squaraine dye followed by a rapid relaxation and subsequent emission. For this ET process, the formation of efficient T-T interactions between the embedded dye and the polymer backbone is important. Thus, the rigidity of F-Sq chromophore probably ensures a close packing with the polymer backbone, whereas the molecular geometry and/or the size of F-Ox and F-PBI avoid such strong π-π interactions, preventing exchange-based ET. As a result, all subsequent sensing studies are focused on mixtures containing F-Sq. It was found that a dye loading of 0.5 wt. % gave the optimal balance between polymer emission and amplification of F-Sq emission, while higher dye loadings showed a decrease of the squaraine emission due to aggregation (see FIG. 25A).

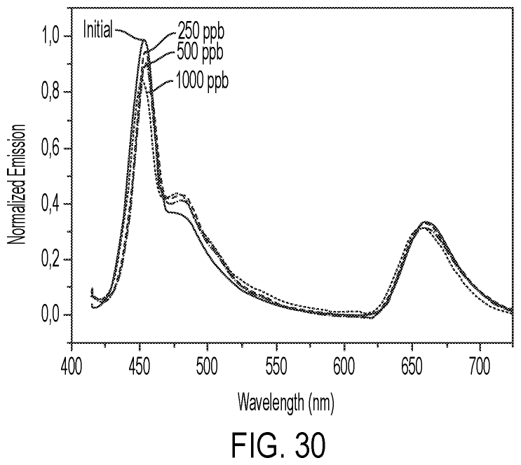

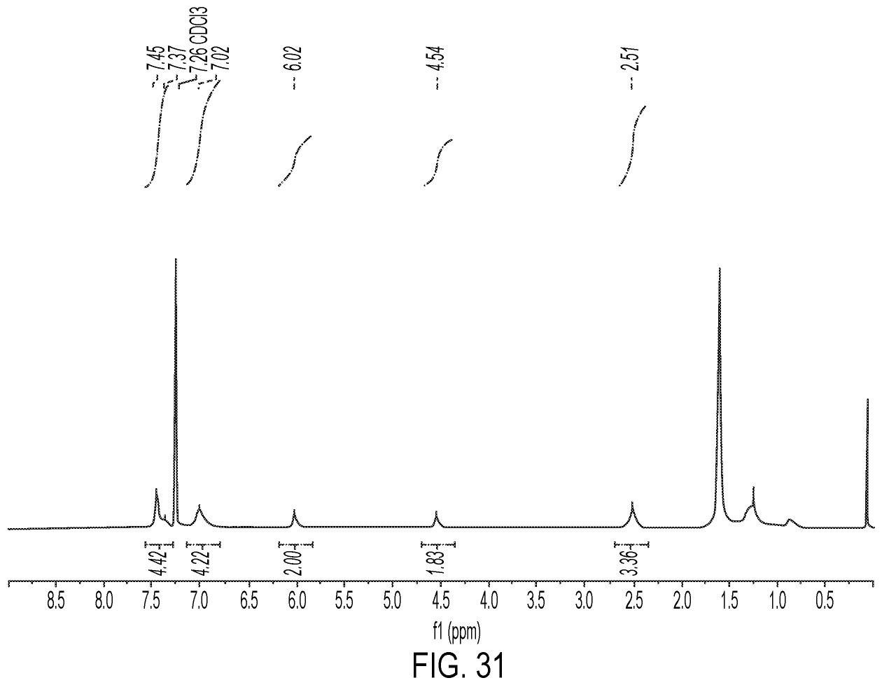

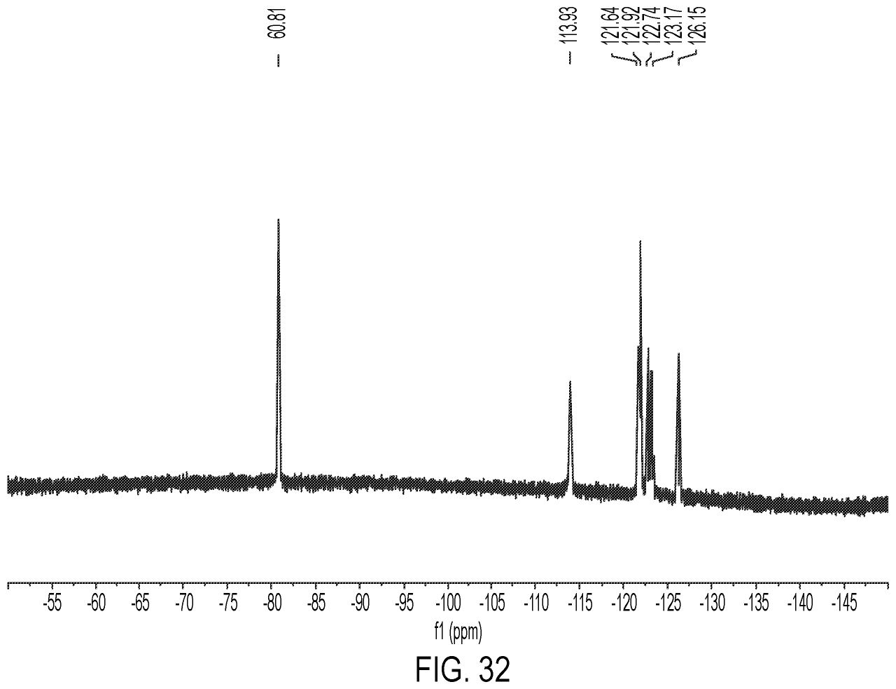

The mechanism of detection is based on an interruption of the ET between the PPE/^{F}PPE polymer and F-Sq, in which the adsorbed PFAS molecules swell the conjugated polymer films and interact with the fluorinated dye, disrupting the polymer-dye π-π interactions that yield exchange-based ET. Therefore, the most relevant time-limiting factor that contributes to the fluorescence response is the time needed for PFAS molecules to diffuse from water to the fluorophilic polymer film. An exposure time of 1 hour was used to perform all the sensing experiments to ensure appropriate PFAS diffusion into the polymer films. Exposure of such thin films (30 to 50 Å thick) to aqueous solutions of PFOA resulted in a decrease of F-Sq emission (“dye-OFF”) and in an increase of PPE/^{F}PPE emission (“polymer-ON”) (see FIG. 25B). Despite the higher fluorine content of ^{F}PPE polymer, it is worth noting that its sensor formulation showed lower fluorescence response than that of PPE polymer. The high hydrophobic nature of ^{F}PPE and the poor wettability of its films probably reduces the diffusion of PFOA molecules from water into the polymer. The limits of detection were calculated to be 174 ppb for PPE/F-Sq formulation, and 412 ppb for ^{F}PPE/F-Sq. Interestingly, PPE films do not exhibit the same fluorescence response when exposed to aqueous solutions of simple octanoic acid (see FIG. 30), thereby demonstrating that the fluorinated segments within the conjugated polymers selectively bind and respond to PFOA.

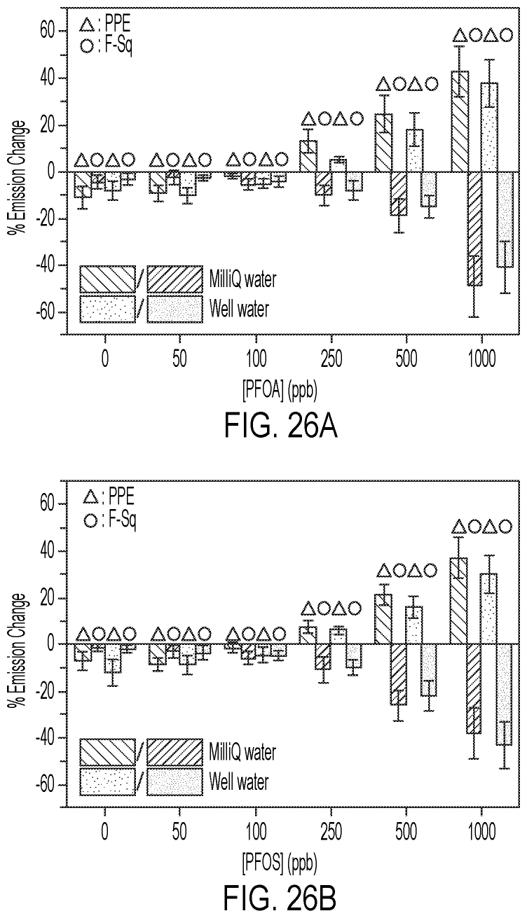

The detection mechanism of the polymer sensors relies on the adsorption of perfluorinated molecules by highly fluorinated polymers, and this triggers ET interruption between the polymer backbone and the dye. Thus, it was wondered if PPE-based formulations could have general utility to sense other PFAS molecules, such as PFOS. As shown in FIGS. 26A-26B, minor deviations in the fluorescence response were observed after exposing PPE/F-Sq films to several concentrations of PFOS, and also the same detection limits within the margin of error (174 ppb for PFOA and 201 ppb for PFOS). Moreover, this non-specific ET interruption-based sensing mechanism may be susceptible to several interferences that are commonly found in complex aqueous matrixes (e.g., ground water). To demonstrate the robustness of the sensors, their performance was evaluated by using a realistic aqueous matrix from a well in Central Vermont. Nonetheless, similar fluorescence responses to PFOA and PFOS were observed, demonstrating that the polymer sensors are not affected by the type of water (see FIGS. 26A-26B).

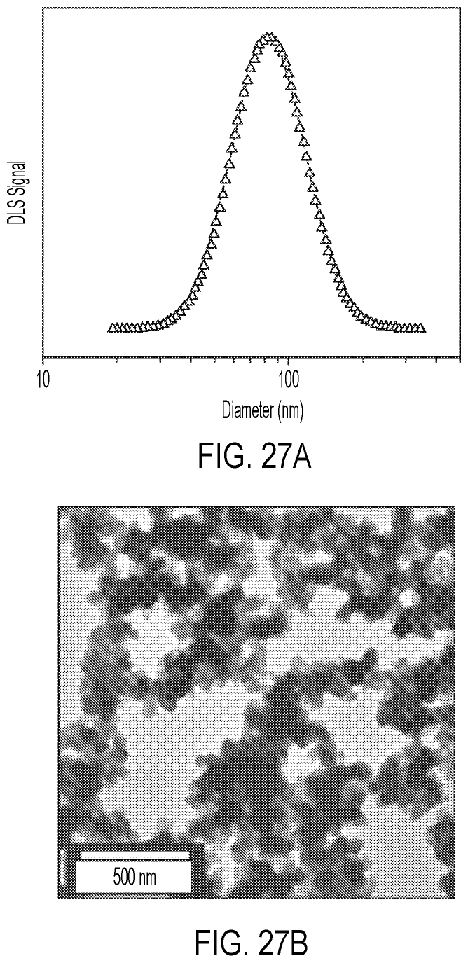

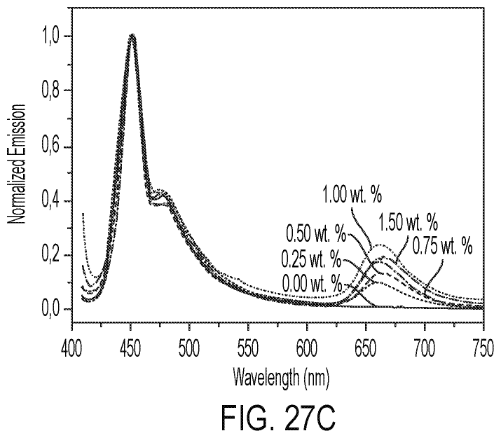

The observed response to PFAS is likely to be highly dependent on the polymer/water interfacial area since the most relevant factor that affects the sensors is the time needed for PFAS to diffuse from water into polymer film. Therefore, it was decided to use conjugated polymer nanoparticle (CPdots) dispersions in water with the intent to increase the polymer/water interfacial area and accelerate PFAS partition into polymers. CPdots aqueous dispersions were prepared by a reprecipitation method, in which a dilute THE solution of the polymer/dye mixture (0.01 mg·mL⁻¹, 2 mL) was quickly added to water (8 mL) under sonication, followed by THE evaporation under vacuum. The obtained aqueous dispersions of CPdots were optically clear and were stable over 1 month with no evidence of precipitation. ^{F}PPE polymer was only soluble in fluorinated solvents, thereby preventing the preparation of CPdots since it requires water-soluble organic solvents, such as THF. As a result, CPdots sensing studies were exclusively focused on PPE/F-Sq formulations. The average size of CPdots was determined by DLS, obtaining monomodal size distributions (PDI<0.20) with mean hydrodynamic diameters of 88 nm (see FIG. 27A). The morphology of the CPdots was investigated by transmission electron microscopy (TEM) (see FIG. 27B). TEM images evidence the presence of spherical nanoparticles that appear to aggregate forming an interconnected network. The formation of such interconnected networks was previously observed for CPdots and is related to the high hydrophobicity of the fluorinated polymer that tends to aggregate during water evaporation.

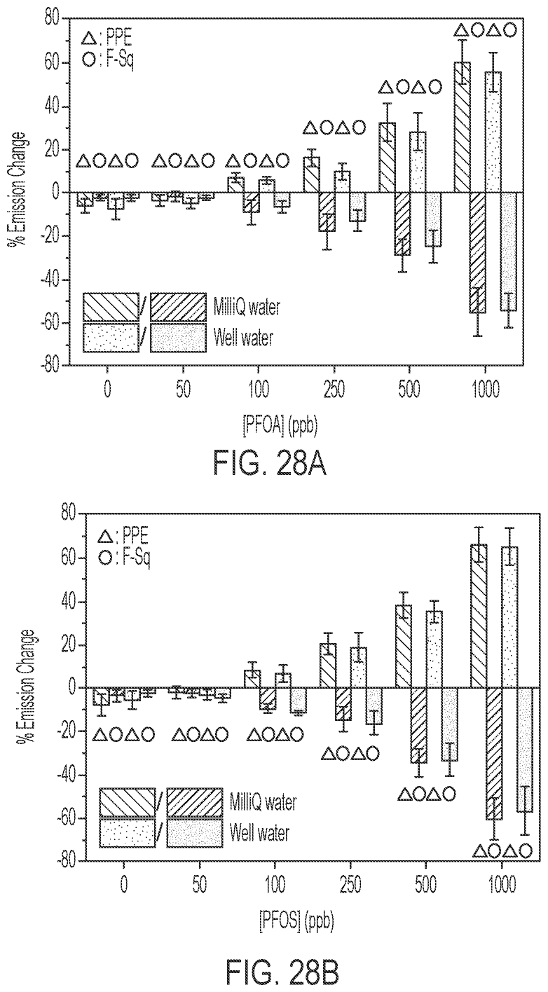

Interestingly, the fluorescence spectra of PPE/F-Sq CPdots were very similar to the spectra acquired in thin film form (see FIG. 27C). This result evidences that F-Sq acceptor also undergoes facile energy transfer when is colocalized in CPdots of PPE polymer, thereby demonstrating that ET via electron exchange mechanism also occurs within CPdots. In this case, a dye loading of 1.0 wt. % was found to give the optimal polymer/dye emission balance. The fluorescence spectra of the CPdots were recorded after 1 hour incubation with different concentrations of PFOA in milliQ and well water. In the same manner as thin film experiments, PFOA exposure resulted in a pseudo-ratiometric “polymer-ON/dye-OFF” response that was not affected by the type of water (see FIG. 28A). CPdots-based sensors are also able to detect PFOS in addition to PFOA (see FIG. 28B). These results confirmed that PFAS molecules are able to diffuse into CPdots and disrupt π-π interactions between F-Sq and PPE, interrupting their ET. A LOD of 43 ppb was calculated for PFOA, and 78 ppb for PFOS. Comparison of thin film and CPdots LODs shows that CPdots aqueous dispersions are more sensitive than thin films, evidencing the impact of a higher surface area in the polymer sensors.

Disclosed herein is a new fluorescent sensing method to selectively detect perfluorooctanoic acid (PFOA) and perfluorooctane sulfonate (PFOS) in aqueous environments at concentrations of μg·L⁻¹. The disclosed method is based on the light-harvesting ability of poly(p-phenylene ethynylene) s to amplify the emission from an embedded dye, as well as the strong distance dependence of the electron exchange-based energy transfer process. To facilitate PFAS partition from water into polymers, highly fluorinated polymers and dyes were designed as the sensing elements. Exposure to aqueous solutions of PFAS produces a small displacement of the dye from the polymer backbone, diminishing the efficiency of the energy transfer and producing a pseudo-ratiometric fluorescent response (“polymer-ON/dye-OFF”). These polymer/dye combinations were evaluated as spin-coated films and as polymer nanoparticles, and were able to selectively detect PFAS at concentrations of ˜200 ppb and ˜50 ppb, respectively. Both polymer films and nanoparticles were not affected by the type of water, and similar responses to PFAS were found in milliQ and well water. These results show an effective sensing approach for on-site detection of aqueous PFAS.

### Materials and Characterization Techniques

Materials: Pentiptycene diacetylene 3 and fluorinated pseudo-pentiptycene diacetylene 2 were prepared following previously reported procedures. Commercial reagents were used as received without further purification: copper (I) iodide, tetrakis (triphenylphosphine)-palladium (0), anhydrous diisopropylamine, perfluorooctanoic acid, perfluorosulfonic acid (Sigma-Aldrich); (perfluorooctyl) propyl iodide, trifluorotoluene (SynQuest); dichloromethane, tetrahydrofuran, methanol, toluene, acetone, hexanes (VWR). Anhydrous toluene was purchased from Sigma-Aldrich and dried using an INERT PureSolv MD5 solvent purification system. Deuterated solvents were purchased from Cambridge Isotope Laboratories and used as received. Milli-Q water was obtained from a Barnstead Nanopure Water System (Thermo Fisher Scientific). Well water was collected from Central Vermont. The well is at 1,400 feet elevation and is in property that abuts the Eastern Boundary of the Green Mountain National Forest.

Instruments: ATR-FTIR spectra were obtained on a Thermo Scientific Nicolet 6700 FTIR spectrophotometer with a Ge crystal for ATR. Solution NMR experiments were carried out on Bruker Avance spectrometers operating at 400 MHz for ¹H and 100 MHz for ¹³C, using standard pulse sequences. Chemical shifts are given in ppm relative to TMS and the residual solvent peak was used as internal reference. Polymer molecular weights were determined at room temperature on a HP series 1100 GPC system in THF at 1.0 mL/min (0.5-1 mg/mL sample concentrations), approximate molecular weights were estimated using a polystyrene calibration standard. High-resolution mass spectra (HRMS) were obtained using a Bruker Autoflex Speed matrix-assisted laser desorption/ionization time-of-flight (MALDI-TOF) spectrometer. Mass spectra were calibrated using poly(ethylene glycol) of the appropriate mass range as external standards.

UV-Vis absorption spectra were recorded on a Cary 60 spectrophotometer and corrected for background signal with a solvent-filled cuvette for solutions and glass slide for thin films.

Fluorescence measurements were performed using a Horiba Quanta-ø fluorescence spectrophotometer, using right-angle detection for solutions and front-face detection for thin films.

Dynamic light scattering (DLS) data for polymer length distribution was obtained from Brookhaven NanoBrook Omni using polymer solutions in benzotrifluoride (0.05 mg·mL⁻¹). The CPdots size and distribution was also obtained from Brookhaven NanoBrook Omni using aqueous dispersions of CPdots (2.5 μg·mL⁻¹). Measurements were performed in three successive 3 min measurements, with no delay between the scans.

Transmission electron microscope (TEM, FEI-Technai) samples were prepared by depositing ˜10 μL of the CPdots dispersions in water (1.25 μg·mL⁻¹) onto carbon film 200 mesh Cu substrates (Electron Microscopy Sciences) and dried to evaporate all water at room temperature.

### Experimental Procedures

Preparation of polymer films: Glass substrates were cleaned by sequential sonication in soapy water, milli-Q water, isopropanol, and followed by UV-ozone treatment. Typically, polymers were dissolved in benzotrifluoride at a concentration of 0.5 mg·mL⁻¹, and filtered using a 0.45 μm filter before applying to the glass substrate. Polymer thin films were deposited on a cover glass (10×10 mm) and spin-casted by a WS-400-6NPP/LITE Spin Processor (Laurell Technologies), using a spin rate of 3000 rpm for 30 s, and placed under vacuum overnight before use.

PFAS sensing with polymer films: The fluorescence response of polymer films to PFOA or PFOS was ascertained by introducing the polymer films into sealed vials (20 mL size) at room temperature containing 2.5 mL of aqueous solutions of PFOA and PFOS. The fluorescence spectra were recorded immediately after exposing the polymer films to the solutions for a specific time (1 hour). The fluorescence studies were performed with excitation wavelength of 400 nm. This procedure was repeated three times with three different polymer films for each PFOA/PFOS concentration. The averages and standard deviations were used to represent each fluorescence intensity data point for each PFOA/PFOS concentration. The limit of detection (LOD) was calculated based on the standard deviation of the response of the curve (o; i.e., the standard deviation of y-intercepts of regression lines) and the slope of the calibration curve(S), according to the formula: LOD=(3.3·σ)/S. σ and S values were obtained from a regression analysis of the calibration curve in Microsoft Excel.

Preparation of polymer nanoparticles (CPdots): The corresponding polymer formulation was dissolved in THF (HPLC grade) at a concentration of 10 μg·mL⁻¹. 2 mL of this solution (previously filtered using a 0.45 μm filter) was added quickly to 8 mL of milliQ water, while sonicating the water. The resulting dispersion was further sonicated for 10 min, and then THF was removed by evaporation in a rotavapor. The resulting nanoparticle dispersions (˜ 2.50 μg·mL⁻¹) were clear, with colors similar to those of the polymers in THF solution.

PFAS sensing with CPdots: The fluorescence response of CPdots to PFOA or PFOS was ascertained by mixing 1.5 mL of the CPdots dispersion and 1.5 mL of the aqueous solutions of PFOA/PFOS into a sealed vial (4 mL size) at room temperature. The fluorescence spectra were recorded immediately after incubating CPdots with PFOA/PFOS for 1 hour. The fluorescence studies were performed with excitation wavelength 400 nm. A total of 3 samples were measured for each individual PFOA/PFOS concentration. The averages and standard deviations were used to represent each fluorescence intensity data point for each PFOA/PFOS concentration. The limit of detection (LOD) was calculated based on the standard deviation of the response of the curve (σ; i.e., the standard deviation of y-intercepts of regression lines) and the slope of the calibration curve(S), according to the formula: LOD=(3.3·σ)/S. σ and S values were obtained from a regression analysis of the calibration curve in Microsoft Excel.

1: 2,5-Diiodobenzene-1,4-diol (1.0 g, 2.76 mmol), 3-(perfluorooctyl) propyl iodide (3.57 g, 6.08 mmol), and potassium carbonate (0.95 g, 6.91 mmol) were stirred in acetone (25 mL). The reaction was stirred at 60° C. for 24 h. The mixture was allowed to cool down to RT and poured into water and extracted twice with ethyl acetate. The combined organic phases were washed with sodium hydroxide 10% (aq.), brine and dried over anhydrous magnesium sulfate. The solution was filtered and the solvent was removed under reduced pressure. The product was purified by flash chromatography on silica gel using DCM/hexanes (1:9). Yield: 83%. IR (v, cm⁻¹): 2885, 1497, 1451, 1355, 1198, 1141, 1049. ¹H NMR (CDCl₃, 400 MHz, δ, ppm): 7.18 (s, 2H), 4.03 (t, J=5.8 Hz, 4H), 2.50-2.30 (m, 4H), 2.18-2.05 (m, 4H). 13C NMR (CDCl₃, 100 MHz, δ, ppm): 152.77, 123.04, 86.36, 68.80, 28.20 (t, J=22.4 Hz) 20.80. ¹⁹F NMR (CDCl₃, 376 MHz, δ, ppm): −80.73, −114.24, −121.63, −121.86, −122.66, −123.42, −126.06. HRMS (MALDI): m/z calcd. for C₂₈H₁₅F₃₄I₂O₂ [M+H]⁺, 1282.8618; found, 1282.8592.

PPE: Under an atmosphere of argon, degassed diisopropylamine/toluene (2:3, 5 mL) solvent was added to a 25-mL Schlenk flask containing dialkyne 3¹ (40.4 mg, 0.084 mmol), diiodide 1 (107.2 mg, 0.084 mmol), copper (I) iodide (0.8 mg, 0.0042 mmol), and tetrakis (triphenylphosphine)-palladium (0) (10 mg, 0.0086 mmol). The flask was deoxygenated by three freeze-pump-thaw cycles and flushed with argon. The reaction mixture was stirred at 80° C. for 72 hours. After cooling down to RT, the polymer precipitates in the solvent mixture. Then, tetrahydrofuran (5 mL) was added to the reaction mixture to dissolve the polymer and was carefully precipitated into methanol.

The solid was re-dissolved in THF, precipitated in methanol and washed with hot methanol, and acetone. Yield: 77%. IR (v, cm⁻¹): 3023, 2970, 1739, 1502, 1460, 1366, 1204, 1230, 1145, 1023, 753, 567. ¹H NMR (CDCl₃, 400 MHz, δ, ppm): 7.58-7.29 (m, 4H), 7.13-6.79 (m, 4H), 6.18-5.52 (m, 2H), 4.69-4.38 (m, 2H), 2.66-2.37 (m, 4H). ¹⁹F NMR (CDCl₃, 376 MHz, δ, ppm): −80.81, −113.93, −121.64, −121.92, −122.74, −123.17, −126.15. GPC (THF, 1 mL·min⁻¹, PS standards): Mn=79.7 kDa, Mw=127.0 kDa, DM=1.60.

^{F}PPE: Under an atmosphere of argon, degassed diisopropylamine/benzotrifluoride (2:3, 5 mL) solvent was added to a 25-mL Schlenk flask containing dialkyne 22 (69.6 mg, 0.042 mmol), diiodide 1 (53.6 mg, 0.042 mmol), copper (I) iodide (0.4 mg, 0.0021 mmol), and tetrakis(triphenylphosphine)-palladium (0) (4.8 mg, 0.0042 mmol). The flask was deoxygenated by three freeze-pump-thaw cycles and flushed with argon. The reaction mixture was stirred at 80° C. for 72 hours. After cooling down to RT, the mixture was diluted with benzotrifluoride (5 mL), and then it was carefully precipitated using methanol. The solid was re-dissolved in benzotrifluoride, precipitated in methanol and washed with hot methanol, and acetone. Yield: 74%. IR (v, cm⁻¹): 2970, 1739, 1506, 1365, 1196, 1143, 736, 703, 657, 521. DLS (Benzotrifluoride, 0.05 mg·mL⁻¹): Polymer length=18.7 nm.

It should be understood that the subject matter defined in the appended claims is not necessarily limited to the specific implementations described above. The specific implementations described above are disclosed as examples only. While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A sensing material, comprising:
a coupled-multichromophore comprising at least one chromophore that displays a change in electromagnetic radiation emission in response to a presence of a fluoroalkyl substance, wherein the coupled-multichromophore is capable of energy transport between individual sites of the coupled-multichromophore.

2. The sensing material of claim 1, wherein the at least one chromophore displays the change in electromagnetic radiation emission in response to protonation of the coupled-multichromophore by the fluoroalkyl substance.

3. The sensing material of claim 1, wherein the coupled-multichromophore comprises at least two chromophores that display a ratiometric change in electromagnetic radiation emission in response to the presence of the fluoroalkyl substance.

4. (canceled)

5. The sensing material of claim 1, wherein the coupled-multichromophore comprises a moiety capable of being protonated by the fluoroalkyl substance.

6. The sensing material of claim 5, wherein the moiety comprises a nitrogen (N)-containing moiety.

7. The sensing material of claim 1, wherein the coupled-multichromophore comprises a pyridine moiety.

8. The sensing material of claim 1, wherein the fluoroalkyl substance is a Brønsted acid.

9. The sensing material of claim 1, wherein the change in electromagnetic radiation emission of the at least one chromophore is a wavelength shift to a lower energy emission.

10. (canceled)

11. The sensing material of claim 1, wherein the coupled-multichromophore comprises a dye.

12. The sensing material of claim 11, wherein the dye comprises a small molecule and/or a polymer.

13. The sensing material of claim 11, wherein the dye comprises squaraine, oxazine, perylene bisimide, conjugates thereof, and/or combinations thereof.

14. (canceled)

15. The sensing material of claim 3, wherein a first chromophore of the at least two chromophores displays an increase in electromagnetic radiation emission in response to the presence of the fluoroalkyl substance, and wherein a second chromophore of the at least two chromophores displays a decrease in electromagnetic radiation emission in response to the presence of the fluoroalkyl substance.

16. The sensing material of claim 1, wherein the fluoroalkyl substance is a per- or poly-fluoroalkyl substance (PFAS).

17. The sensing material of claim 1, wherein the coupled-multichromophore comprises a polymer and/or an assembly of small molecules.

18. (canceled)

19. The sensing material of claim 1, wherein the coupled-multichromophore comprises a polyarylene, a poly(arylene vinylene), a poly(thiophene), a poly(phenylene), a poly(fluorene), a poly(phenylene), a poly(arylene ethynylene), a poly(phenylene ethynylene), copolymers thereof, and/or combinations thereof.

20. (canceled)

21. The sensing material of claim 1, wherein the coupled-multichromophore is at least partially fluorinated.

22. (canceled)

23. The sensing material of claim 1, wherein the coupled-multichromophore comprises fluorine in an amount greater than or equal to 25 weight percent (wt. %) versus a total weight of the coupled-multichromophore.

24. (canceled)

25. The sensing material of claim 1, wherein the sensing material is capable of detecting the presence of the fluoroalkyl substance at a sensitivity less than or equal to 200 parts per billion (ppb).

26-27. (canceled)

28. The sensing material of claim 1, wherein the sensing material comprises a film comprising the coupled-multichromophore.

29. The sensing material of claim 1, wherein the sensing material comprises a plurality of particles comprising the coupled-multichromophore.

30. The sensing material of claim 1, wherein the sensing material is configured to absorb the fluoroalkyl substance.

31. The sensing material of claim 1, wherein a quantum yield of the coupled-multichromophore is greater than or equal to 30%.

32. (canceled)

33. A method of detecting a fluoroalkyl substance, comprising:
exposing a coupled-multichromophore to a solution comprising the fluoroalkyl substance; and
detecting a change in electromagnetic radiation emission of at least one chromophore of the coupled-multichromophore, wherein the at least one chromophore displays the change in electromagnetic radiation emission in response to a presence of the fluoroalkyl substance.

34-113. (canceled)
